(19) United States
(12) Patent Application Publication
(10) Pub. No.: US 2025/0197905 A1
(43) Pub. Date: Jun. 19, 2025

(54) LEGIONAMINIC ACID GLYCOSYLTRANSFERASES FOR CHEMOENZYMATIC SYNTHESIS OF GLYCANS AND GLYCOCONJUGATES

(71) Applicant: The Regents of the University of California, Oakland, CA (US)

(72) Inventors: Xi CHEN, Davis, CA (US); Xiaohong YANG, Davis, CA (US); Hai YU, Davis, CA (US); Bijoyananda MISHRA, Davis, CA (US)

(21) Appl. No.: 18/848,017

(22) PCT Filed: Mar. 20, 2023
(86) PCT No.: PCT/US2023/064726

§ 371 Date: Sep. 17, 2024

Related U.S. Application Data
(60) Provisional application No. 63/321,743, filed on Mar. 20, 2022.

(51) Int. Cl. C12P 19/04; C12N 9/12; C12N 9/88

(52) U.S. Cl. CPC C12P 19/04; C12N 9/1241; C12N 9/88; C07K 2319/24; C12Y 207/07043; C12Y 207/07082; C12Y 401/03003

(57) ABSTRACT

Provided herein are methods for preparing a glycan product containing legionaminic acid moieties and other nonulosonic acids. Also provided herein are legionaminic acid transferase fusion proteins and vaccine compositions containing glycan products prepared according to the described methods.

### CROSS-REFERENCE TO RELATED APPLICATION

The present application claims priority to U.S. Provisional Pat. Appl. No. 63/321,743, filed on Mar. 20, 2022, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under Grant. No. RO1GM141324 awarded by the National Institutes of Health. The government has certain rights in the invention.

### REFERENCE TO BIOLOGICAL SEQUENCE DISCLOSURE

The instant application contains a Sequence Listing which has been submitted in .xml format via Patent Center in accordance with 37 C.F.R. §§ 1.831 to 1.834, and is hereby incorporated by reference in its entirety for all purposes. The Sequence Listing written in .xml format is named SL-081906-1379792.xml, was created on Mar. 20, 2023, and is 18,262 bytes in size.

### BACKGROUND OF THE INVENTION

Nonulosonic acids (NulOs) are 9-carbon monosaccharides containing α-keto acid functional groups. The most well-known members are sialic acids (Sias) that have been found in vertebrates and some pathogenic bacteria. More recently, a diverse array of bacterium-specific nonulosonic acids have been identified. These include legionaminic acids (Legs) such as the most well studied di-N-acetyllegionaminic acid form, 5,7-diacetamido-3,5,7,9-tetradeoxy-D-glycero-D-galacto-non-2-ulosonic acid (Leg5,7Ac₂), as well as 4-epi-legionaminic acid, 8-epi-legionaminic acid, pseudaminic acid (Pse), acinetaminic acid, fusaminic acid, and their derivatives. Leg5,7Ac₂ was first identified in 1994 as the building block of its α2-4-linked homopolymer constituting the O-antigen of Legionella pneumophila serogroup 1, the Gram-negative bacterium responsible for Legionnaires' disease. Leg5,7Ac₂ and derivatives varying in N-substitutions at C-5 and/or C-7 have since been reported for the lipopolysaccharides (LPSs) and capsular polysaccharides (CPSs) of various other important bacterial pathogens, such as Acinetobacter baumannii, Campylobacter jejuni, Cronobacter turicensis, Enterobacter cloacae, Escherichia coli, and others. They have also been identified in the glycoproteins of Halorubrum (Hrr.) sp. pleomorphic virus 1 (HRPV-1) and the flagella of Campylobacter jejuni.

LPSs of Gram-negative bacteria and CPSs of both Gram-negative and Gram-positive bacteria often constitute the outermost layer of the bacterial cells and are involved in mediating interactions between the bacteria, their environment, and host immune system. CPSs and LPSs have been implicated as important virulence factors for many bacterial pathogens, including those expressing Leg5,7Ac₂ or derivatives. Glycoconjugate vaccines based on bacterial surface polysaccharides have been proven effective in combatting battles against bacterial infection.

### BRIEF SUMMARY OF THE INVENTION

Provided herein are methods for preparing a glycan product containing a nonulosonic acid moiety (e.g., a legionaminic acid moiety), the method comprising: (i) forming a reaction mixture comprising a legionaminic acid transferase (LegT), a donor comprising a nonulosonic acid moiety, and a glycan acceptor, and (ii) maintaining the reaction mixture under conditions for LegT-catalyzed transfer of the nonulosonic acid moiety from the donor to the glycan acceptor, thereby forming the glycan product containing the nonulosonic acid moiety. Also provided herein are legionaminic acid transferase fusion proteins, isolated nucleic acids encoding legionaminic acid transferase fusion proteins, and vectors and host cells comprising the isolated nucleic acids as described herein. Also provided are glycan products and vaccine compositions containing glycan products prepared according to the methods described herein.

### BRIEF DESCRIPTION OF THE DRAWINGS

FIG. 1 shows the structures of legionaminic acid (Leg) and 5,7-diacetamido-3,5,7,9-tetradeoxy-D-glycero-D-galacto-non-2-ulosonic acid (5,7-di-N-acetyllegionaminic acid or Leg5,7Ac₂).

FIG. 2 shows the SDS-PAGE analysis results of the expression and purification of recombinant enzymes. Lanes: M1, THERMO SCIENTIFIC™ PAGERULER™ Prestained Protein Ladder (10-180 kDa); M2, THERMO SCIENTIFIC™ PAGERULER™ Plus Prestained Protein Ladder (10-250 kDa); BI, whole cell extract before induction, AI, whole cell extract after induction; L, lysate after induction; P, Ni²⁺-NTA column purified protein.

FIG. 3A shows a pH profile for AbGtr18 activity.

FIG. 3B shows a temperature profile for AbGtr18 activity.

FIG. 3C shows the effects of divalent metal ions, EDTA, and DTT on the activity of AbGtr18.

FIG. 3D shows the effects of Mg²⁺ concentration on the activity of AbGtr18.

FIG. 3E shows time-course studies of AbGtr18 activity.

FIG. 3F shows a thermostability profile for AbGtr18.

FIG. 4 shows a nonulosonic acid-containing glycan library prepared using AbGtr18.

FIG. 5 shows a time-course study of AbGtr18 activity with CMP-Leg5,7Ac₂ and CMP-Neu5Ac, generated in situ.

FIG. 6 shows nonlimiting exemplary glycan acceptors used in the methods described herein.

FIG. 7 shows nonlimiting exemplary glycan products prepared according to the methods described herein.

FIG. 8 shows ¹H (400 MHz, D₂O) and ¹³C (100 MHz, D₂O) NMR spectra of GalNAcαProNHCbz.

FIG. 9 shows ¹H (600 MHz, D₂O) and ¹³C (150 MHz, D₂O) NMR spectra of Neu5Acα2-6GalNAcαProNHCbz.

FIG. 10 shows ¹H (600 MHz, D₂O) and ¹³C (150 MHz, D₂O) NMR spectra of Neu5Acα2-6GalNAc.

FIG. 11 shows ¹H (400 MHz, CDCl₃) and ¹³C (100 MHz, CDCl₃) NMR spectra of compound 1.

FIG. 12 shows ¹H (400 MHz, CDCl₃) and ¹³C (100 MHz, CDCl₃) NMR spectra of compound 2.

FIG. 13 shows ¹H (400 MHz, CDCl₃) and ¹³C (100 MHz, CDCl₃) NMR spectra of compound 3.

FIG. 14 shows ¹H (400 MHz, D₂O) and ¹³C (100 MHz, D₂O) NMR spectra of compound 4.

FIG. 15 shows ¹H (400 MHz, D₂O) and ¹³C (100 MHz, D₂O) NMR spectra of compound 5.

FIG. 16 shows ¹H (400 MHz, CDCl₃) and ¹³C (100 MHz, CDCl₃) NMR spectra of compound 6.

FIG. 17 shows ¹H (400 MHz, CDCl₃) and ¹³C (100 MHz, CDCl₃) NMR spectra of compound 7.

FIG. 18 shows ¹H (400 MHz, D₂O) and ¹³C (100 MHz, D₂O) NMR spectra of compound 8.

FIG. 19 shows ¹H (400 MHz, CDCl₃) and ¹³C (100 MHz, CDCl₃) NMR Spectra of compound 9.

FIG. 20 shows ¹H (400 MHz, D₂O) and ¹³C (100 MHz, D₂O) NMR Spectra of compound 10.

FIG. 21 shows ¹H (600 MHz, D₂O) and ¹³C (150 MHz, D₂O) NMR spectra of Leg5,7Ac₂α2-6GalNAcαProNHCbz.

FIG. 22 shows ¹H (600 MHz, D₂O) and ¹³C (150 MHz, D₂O) NMR spectra of Leg5Ac₇N₃α2-6GalNAcαProNHCbz.

### DETAILED DESCRIPTION OF THE INVENTION

The present disclosure provides new methods for preparation of glycan products, employing legionaminic acid-glycosyltransferases such as, but not limited to, AbGtr18 from A. baumannii K8. As described below, AbGtr18 shows high efficiency (>99%) and specificity towards GalNAcαOR acceptor substrates. Donor and acceptor substrate selectivity and promiscuity allow efficient synthesis of glycans and glycoconjugates containing legionaminic acids, sialic acids, and their derivatives. For example, Leg- and Sia-terminated glycans including sialyl Tn (STn) antigen (which is associated with an adverse outcome and poor prognosis in cancer patients) can be produced in high yields.

Provided herein are methods for preparing a glycan product containing a nonulosonic acid moiety. The methods include:
(i) forming a reaction mixture comprising a legionaminic acid transferase (LegT), a donor comprising a nonulosonic acid moiety, and a glycan acceptor, and
(ii) maintaining the reaction mixture under conditions for LegT-catalyzed transfer of the nonulosonic acid moiety from the donor to the glycan acceptor, thereby forning the glycan product containing the nonulosonic acid moiety.

In some embodiments, the LegT is a Legionella LegT, an Acinetobacter LegT, a Campylobacter LegT, a Cronobacter LegT, an Enterobacter LegT, or an Escherichia LegT.

In some embodiments, the LegT is an L. pneumophila LegT, an A. baumannii LegT, a C. jejuni LegT, a C. turicensis LegT, an E. cloacae LegT, or an E. coli LegT.

In some embodiments, the LegT comprises a polypeptide having at least 80% sequence identity to SEQ ID NO:1 (A. baumannii K8 AbGtr18), SEQ ID NO:2 (A. baumannii K27 AbGtr56K27), SEQ ID NO:3 (A. baumannii K44 AbGtr56K44), SEQ ID NO:4 (A. baumannii K54 AbGtr109), SEQ ID NO:5 (C. jejuni 108 CjMaf4), SEQ ID NO:6 (C. turicensis G3882 CtWepA), SEQ ID NO:7 (E. coli O161 EcWeiF), or SEQ ID NO:8 (E. coli O61 EcWeiK).

In some embodiments, the LegT comprises a polypeptide having at least 95% sequence identity to SEQ ID NO:1 (A. baumannii K8 AbGtr18).

The amino acid sequence of a particular enzyme may have, for example, at least about 70%, e.g., at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 910%, at least about 92%, at least about 93%, at least about 94%, at least about 95%, at least about 96%, at least about 97%, at least about 98%, or at least about 99% sequence identity to any one of the amino acid sequences set forth herein (e.g., in SEQ ID NOS:1-8).

“Identical” and “identity,” in the context of two or more polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)). Additional examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available, for example, at the National Center for Biotechnology Information website. The BLAST algorithm parameters W, T, and X determine the sensitivity and speed of the alignment. For amino acid sequences, the BLASTP program uses as defaults a word size (W) of 3, an expectation (E) of 10, and the BLOSUM62 scoring matrix (see, e.g., Henikoff and Henikoff, Proc. Nat. Acad Sci. USA 89:10915 (1989)).

In some embodiments, the LegT may include one or more heterologous amino acid sequences located at the N-terminus and/or the C-terminus of the enzyme. The LegT may contain a number of heterologous sequences that are useful for expressing, purifying, and/or using the enzyme. The LegT can contain, for example, a poly-histidine tag (e.g., a His₆ tag, SEQ ID NO:13); a calmodulin-binding peptide (CBP) tag; a NorpA peptide tag; a Strep tag for recognition by/binding to streptavidin or a variant thereof, a FLAG peptide for recognition by/binding to anti-FLAG antibodies (e.g., M1, M2, M5); a glutathione S-transferase (GST); or a maltose binding protein (MBP) polypeptide. In some embodiments, the N-terminus of the LegT polypeptide is fused to a maltose binding protein.

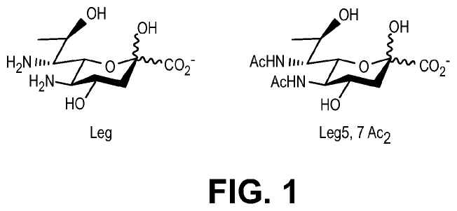

In some embodiments, the nonulosonic acid moiety is a legionaminic acid selected from the group consisting of 5,7-diacetamido-3,5,7,9-tetradeoxy-D-glycero-D-galacto-non-2-ulosonic acid (Leg5,7Ac₂), 4-epi-legionaminic acid, 8-epi-legionaminic acid, Leg5Ac7Ac4OAc, Leg5Ac7Hb, pseudaminic acid (Pse), Pse5Ac7Ac, Pse5Ac7Ac4OAc, Pse5Ac7Hb, acinetaminic acid, and fusaminic acid, wherein Ac is acetyl and Hb is 3-hydroxybutanoyl. In some embodiments, the legionaminic acid is Leg5,7Ac₂, as shown in FIG. 1.

In some embodiments, the nonulosonic acid is a sialic acid selected from the group consisting of neuraminic acid, Neu5Ac, Neu5Gc, and Kdn.

In some embodiments, the nonulosonic acid moiety is selected from the group consisting of Leg5,7Ac₂, Leg5,7diN₃, Neu5Ac, Neu5Gc, Kdn, Neu5Az, Neu5Ac9N₃, Neu5Ac7N₃, Neu5,7Ac₂, Neu5,7diN₃, Neu5,7,9triN₃, Leg5Ac7N₃, and Leg5N₃7Ac.

In some embodiments, the donor is selected from the group consisting of CMP-Leg5,7Ac₂, CMP-Leg5,7diN₃, CMP-Neu5Ac, CMP-Neu5Gc, CMP-Kdn, CMP-Neu5Az, CMP-Neu5Ac9N₃, CMP-Neu5Ac7N₃, CMP-Neu5,7Ac₂, CMP-Neu5,7diN₃, CMP-Neu5,7,9triN₃, CMP-Leg5Ac7N₃, and CMP-Leg5N₃7Ac.

In some embodiments, the nonulosonic acid moiety is a legionaminic acid, and wherein forming the reaction mixture comprises combining a the legionaminic acid with cytidine 5′-triphosphate and a CMP-legionaminic acid synthetase (CLS) to form the donor.

In some embodiments, the CLS is Legionella pneumophila CMP-Leg5,7Ac₂ synthetase (LpCLS).

In some embodiments, forming the reaction mixture further comprises combining a nonulosonic acid precursor 6-deoxy-manosamine, an N-acetyl-6-deoxy-mannosamine, or an azido-6-deoxymannose with pyruvate and a sialic acid aldolase to form the legionaminic acid.

In some embodiments, the nonulosonic acid precursor is selected from the group consisting of 6deoxyMan2,4diNAc, 6deoxyMan2N₃4NAc, 6deoxyManNAc4N₃, and Man2,4diNAc.

In some embodiments, the sialic acid aldolase is Pasteurella multocida sialic acid aldolase (PmAldolase).

In some embodiments, the nonulosonic acid moiety is a sialic acid, and wherein forming the reaction mixture comprises combining the sialic acid with cytidine 5′-triphosphate and a CMP-sialic acid synthetase (CSS) to form the donor.

In some embodiments, the CSS is Neisseria meningitidis CMP-sialic acid synthetase (NmCSS).

In some embodiments, forming the reaction mixture further comprises combining a nonulosonic acid precursor mannose, a mannosamine, an N-acetylmannosamine, or an azido-mannose with pyruvate and a sialic acid aldolase to form the sialic acid.

In some embodiments, the nonulosonic acid precursor is selected from the group consisting of ManNAc, ManNGc, Mannose, ManNAz, ManNAc6N₃, ManNAc4N₃, Man2,4diN₃, Man2,4,6triN₃, 6deoxyMan2,4diN₃, and 6deoxyManNAc4N₃.

In some embodiments, the sialic acid aldolase is Pasteurella multocida sialic acid aldolase (PmAldolase).

A number of suitable glycan acceptors (also referred to herein as “acceptor glycans”) may be used in the methods provided herein. In some embodiments, the glycan acceptor contains a hexose (e.g., N-acetyl galactosamine) or a hexose covalently bonded to a monosaccharide, an oligosaccharide, a polysaccharide, an amino acid, an oligopeptide, a polypeptide, a lipid, or another synthetic handle. In some embodiments, the glycan acceptor is a disaccharide, a trisaccharide, a tetrasaccharide, a pentasaccharide, a hexasaccharide, a heptasaccharide, an octasaccharide, a nonasaccharide, or a decasaccharide. In some embodiments, the glycan acceptor is an octasaccharide, a nonasaccharide, or a decasaccharide.

In some embodiments, the glycan acceptor is an N-acetylgalactosamine or an N-acetylgalactosamine-terminated glycoside “GalNAc-OR,” wherein R is H, a monosaccharide, an oligosaccharide, a polysaccharide, a peptide, a protein, a lipid, a glycopeptide, a glycoprotein, or a glycolipid. In some embodiments, the N-acetylgalactosamine is linked to OR via an α-linkage at the anomeric carbon C-1 of GalNAc.

The glycan acceptor may contain a purification handle, e.g., a hydrophobic moiety such as a perfluorinated alkyl group or a fatty acid moiety as described, for example, in WO 2014/201462. Products containing the purification handle may be separated from reaction mixtures via reverse phase chromatography, solid phase extraction, or like techniques. Purification handles may also include chromophores (e.g., aromatic substituents such as benzyloxycarbonyl) to aid in identification and purification of desired products. In some embodiments, the purification handle includes an (N-benzyloxycarbonyl)aminopropyl moiety. In some embodiments, the acceptor sugar has the structure:

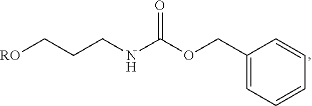

wherein R is a monosaccharide or an oligosaccharide.

Following purification, the benzyloxycarbonyl moiety may be removed (e.g., by combination with an acid such a formic acid or trifluoroacetic acid or by catalytic hydrogenation) to provide an aminopropyl moiety —(CH₂)₃NH₂ at the reducing end of the glycan product. The aminopropyl moiety, in turn may serve as conjugation handle for covalent coupling to a carrier material, e.g., in a vaccine composition.

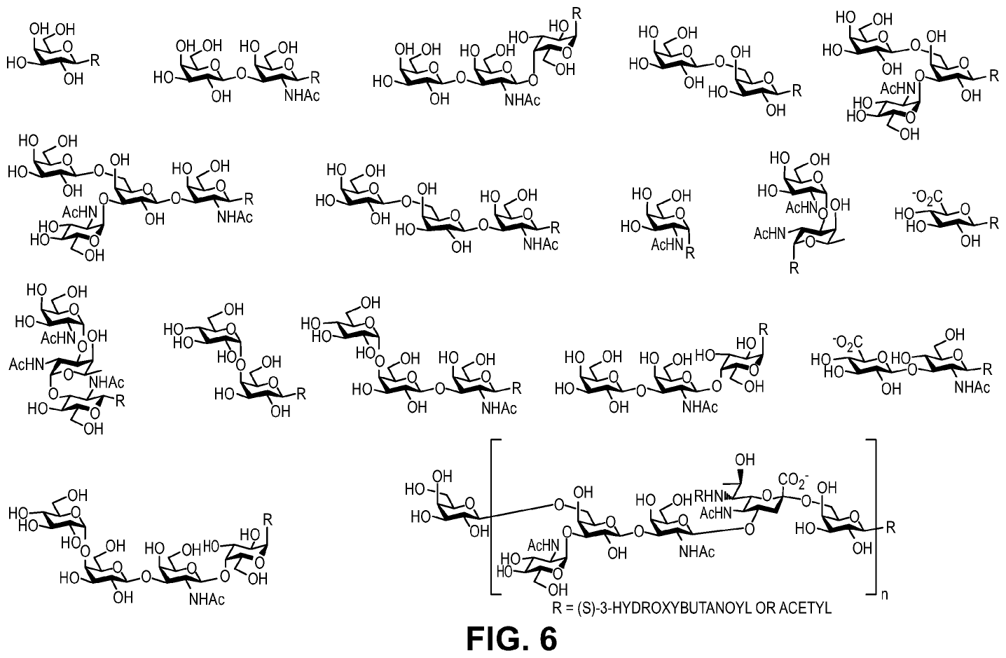

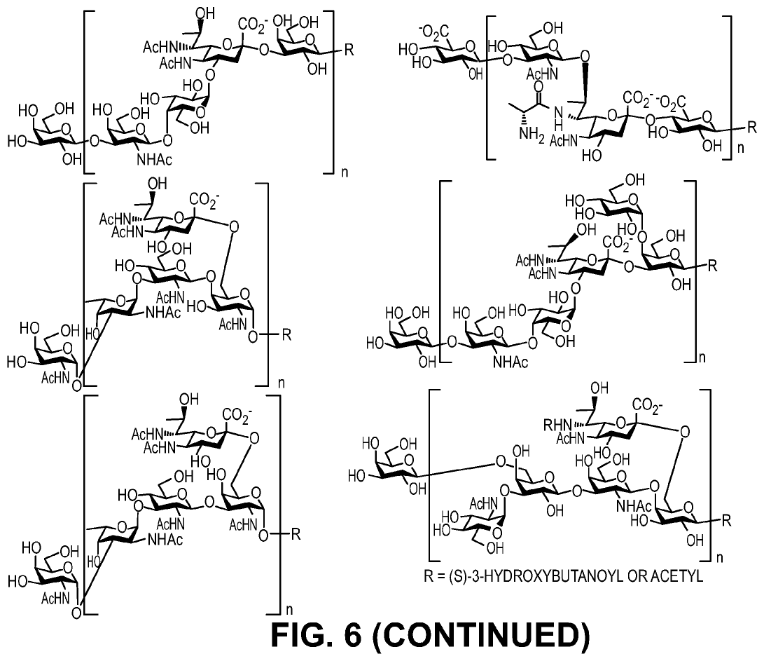

In some embodiments, the glycan acceptor comprises a methyl 2-anthranilic acid ester moiety, a benzyloxycarbonyl-propylamine moiety, or 4-nitrophenyl moiety at the reducing end of the glycan acceptor. In some embodiments, the glycan acceptor comprises one or more structures depicted in FIG. 6, wherein “n” is from 0 to 6 (i.e., 0, 1, 2, 3, 4, 5, or 6), and optionally comprises a purification handle (e.g., a benzyloxycarbonyl-propylamine moiety) at the reducing end of the glycan acceptor.

In some embodiments, non-limiting examples of the glycan acceptor include:

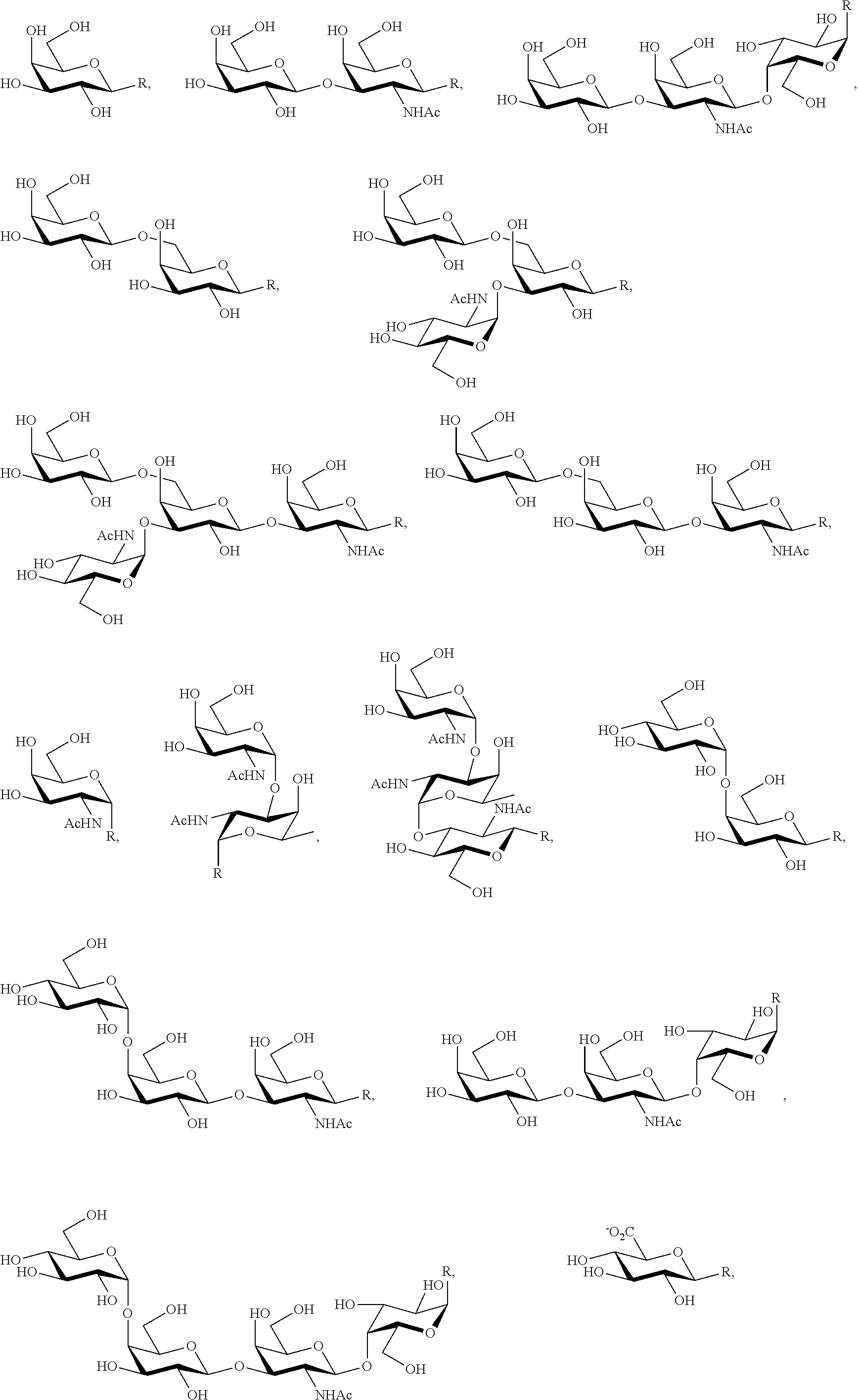

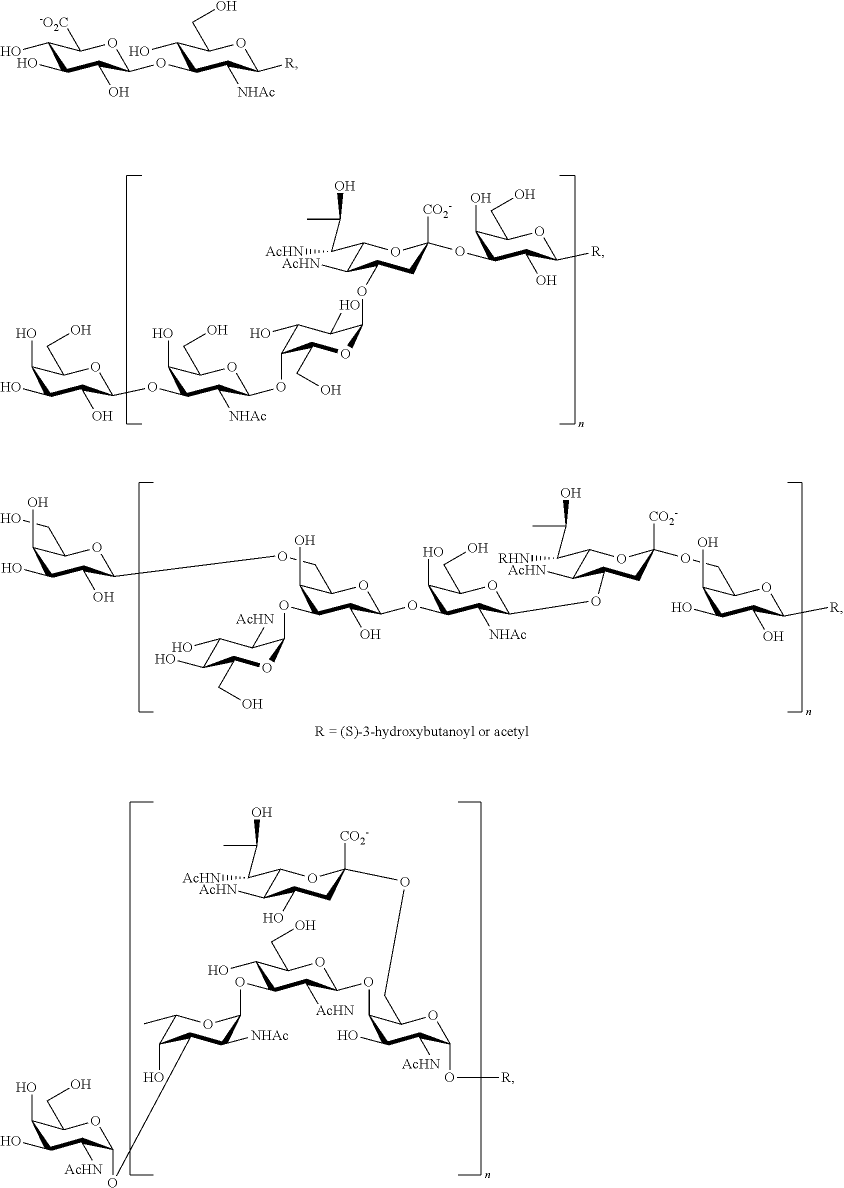

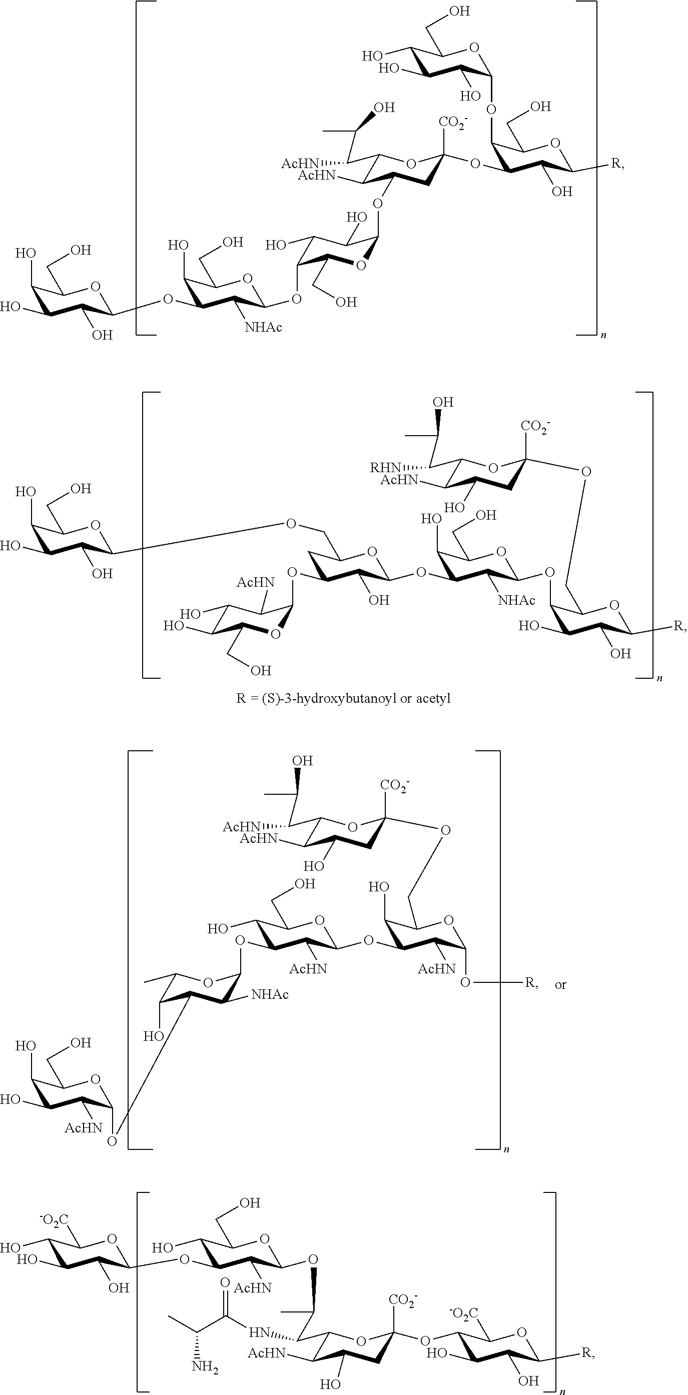

wherein “n” is from 0 to 6 (i.e., 0, 1, 2, 3, 4, 5, or 6), and optionally comprises a purification handle (e.g., a benzyloxycarbonyl-propylamine moiety) at the reducing end of the glycan acceptor.

In some embodiments, the glycan acceptor and/or the glycan product comprises one or more L-FucNAc moieties. In some such embodiments, the reaction mixture may include additional enzymes such as 4,6-dehydratase/3,5-epimerase (including but not limited to AbFn1A; e.g.. Uniprot Accession No. V5RCZ1, A0A0H4UV01, V5RBP5, A0A2U9QFI4, A0A2S1WLP2, A0A097I586, or A0A481WWC0), reductase (AbFn1B; e.g., Uniprot Accession No. N0AAT6, A0A0H4UR15, V5RDZ1, A0A2U9QFI5, A0A2S1WLX4, or A0A09715A5), and 2-epimerase (including but not limited to AbFn1C; e.g., Uniprot Accession No. A0A097I5C2, N0A608, V5RD04, A0A2U9QFX2, A0A2S1WLS4, A0A222UQX4, A0A0U4DKF6, A0A481WWL7, or A0A481WXY1) in multiple gene clusters of bacteria such as A. baumannii. The reaction mixture can contain further glycosyltransferases and other enzymes (including, but not limited to, kinases, dehydrogenases, pyrophosphatases, nucleotide sugar synthetases) as described, for example, in WO 2020/163784, which is incorporated herein by reference in its entirety.

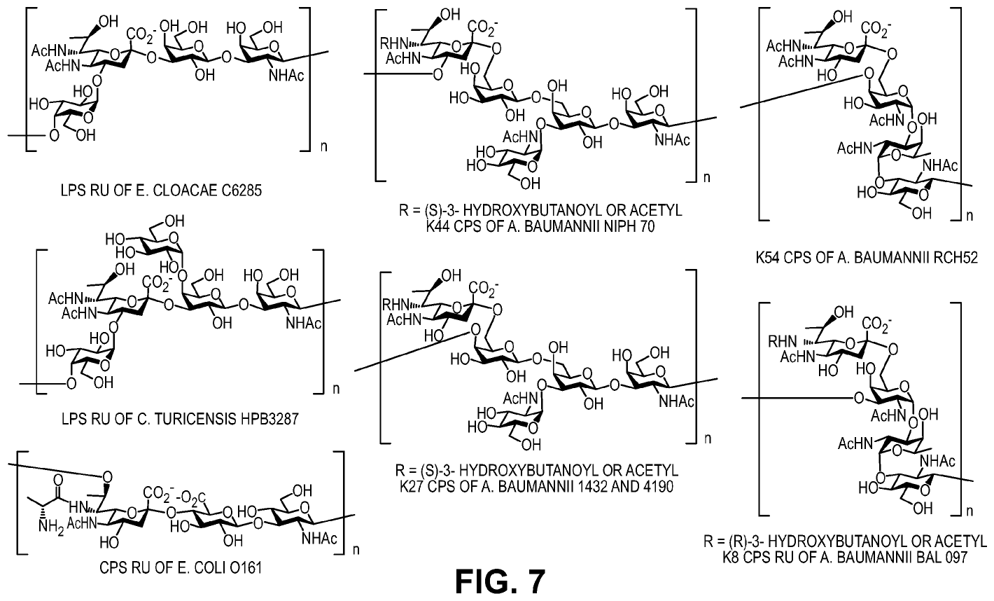

In some embodiments, the glycan product comprises one or more A. baumannii bacterial capsular polysaccharide K2 units, K6 units, K7 units, K8 units, K16 units, K27 units, K33 units, K42 units, K44 units, K46 units, K54 units, K63 units, K90 units, or K93 units. Such bacterial capsular polysaccharides and subunits are described for example, by Kenyon et al. [Glycobiology. 2014, 24(6): 554-63; Carbohydr. Res. 2015, 409:30-5; Int J Biol Macromol. 2019, 128: 101-6; PLoS ONE 14(6): e0218461], Senchenkova et al. [Carbohydr. Res. 2019, 479:1-5], Arbatsky et al. [Russian Chemical Bulletin. 2016, 65(2):588-91; Russ Chem Bull. Int. Ed. 2019, 68(1): 163-7; Carbohydr. Res. 2019, 483:107745], Kasimova et al. [Biochem (Moscow). 2017, 82(4): 483-9], Shashkov et al. [Russ Chem Bull. 2015, 64(5): 1196-1199; Glycobiology. 2016; 26(5): 501-8], Senchenkova et al. [Carbohydr. Res. 2015, 407:154-7], and Haseley et al. [Eur J Biochem. 1997, 250:617-23], which references are incorporated herein by reference in their entireties. In some embodiments, the glycan product comprises one or more A. baumannii bacterial capsular polysaccharide depicted in FIG. 7, wherein “n” is 1, 2, 3, 4, 5, or 6, and optionally comprises a purification handle (e.g., a benzyloxycarbonyl-propylamine moiety) at the reducing end of the glycan product. In some embodiments, the glycan product is Galα1-4Leg5,7Ac₂α2-3Galβ1-3GalNAcβProNH2. In some embodiments, the glycan product is GalNAcβ1-4Galα1-4Leg5,7Ac₂α2-3GalβProNH₂. In some embodiments, the glycan product is Galβ1-3GalNAcβ1-4Galα1-4Leg5,7Ac₂αProNH₂. In some embodiments, the glycan product is Leg5,7Ac₂α2-3Galβ1-3GalNAcβ1-4GalαProNH₂. In some embodiments, the glycan product is Galα1-4Leg5,7Ac₂α2-3(Glcα1-4)Galβ1-3GalNAcβProNH₂. In some embodiments, the glycan product is Leg5Ac7Rα2-6Galβ1-6(GlcNAcα1-3)Galβ1-3GalNAcβProNH₂, wherein R is acetyl or (S′)-3-hydroxybutanoyl. In some embodiments, the glycan product is Leg5Ac7Rα2-6GalNAcα1-3-L-FucNAcα-3GcNAcβProNH₂, wherein R is acetyl or (R)-3-hydroxybutanoyl. In some embodiments, the glycan product is Leg5Ac7Rα2-4GlcAβ1-3GlcNAcβProNH₂, wherein R is D-alanyl.

In some embodiments, non-limiting examples of the glycan product include one or more A. baumannii bacterial capsular polysaccharide:

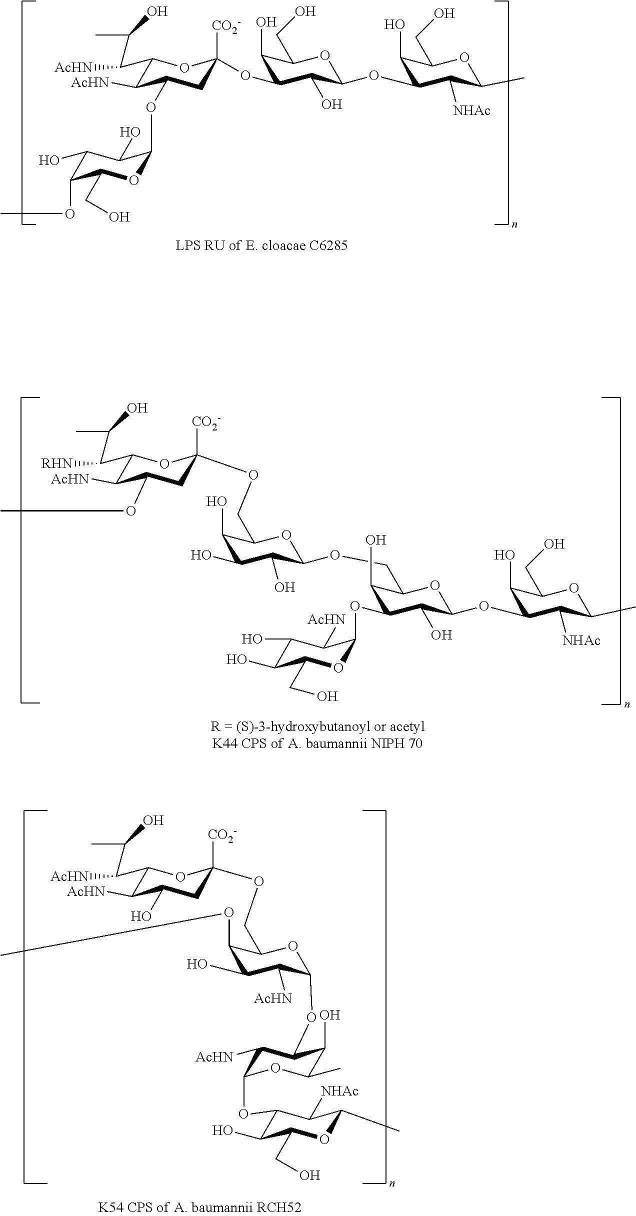

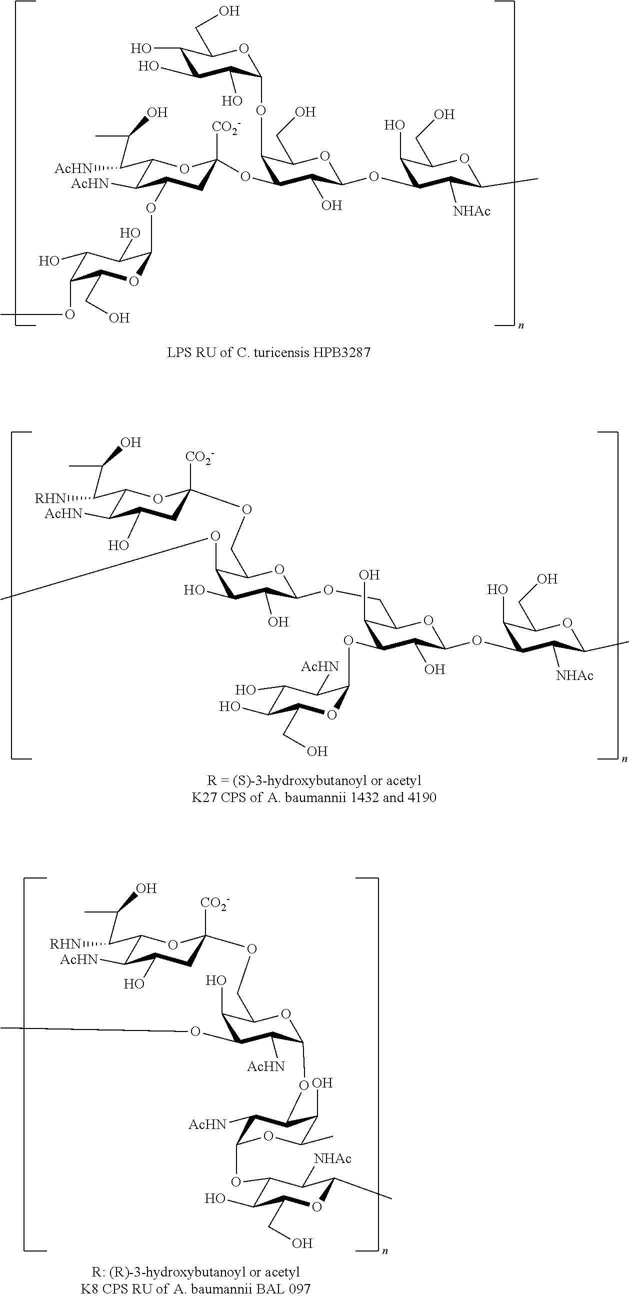

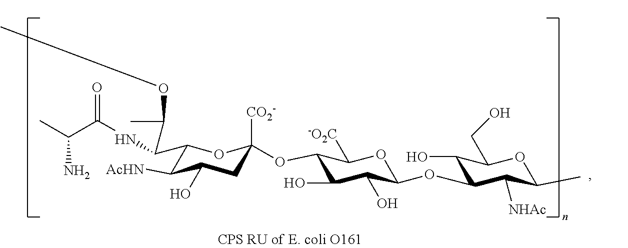

wherein “n” is from 1 to 6 (i.e., 1, 2, 3, 4, 5, or 6), and optionally comprises a purification handle (e.g., a benzyloxycarbonyl-propylamine moiety) at the reducing end of the glycan product.

The methods generally include providing reaction mixtures that contain at least one LegT, a glycan acceptor, and one or more sugar donors. LegTs and other enzymes can be, for example, isolated or otherwise purified prior to addition to the reaction mixture. As used herein, a “purified” enzyme refers to an enzyme which is provided as a purified protein composition wherein the enzyme constitutes at least about 50% of the total protein in the purified protein composition. For example, the enzyme can constitute about 50%, 60%, 70%, 75%, 80%, 85%, 90%, 95%, or 99% of the total protein in the purified protein composition. The amount of enzyme in a purified protein composition can be determined by any number of known methods including, for example, by polyacrylamide gel electrophoresis (e.g., SDS-PAGE) followed by detection with a staining reagent (e.g., Coomassie Brilliant Blue G-250, a silver nitrate stain, and/or a reagent containing a capsular polysaccharide antibody). The LegTs and other enzymes used in the methods can also be secreted by a cell present in the reaction mixture. Alternatively, a LegT or other enzyme can catalyze the reaction within a cell expressing the enzyme.

Reaction mixtures can contain additional reagents for use in glycosylation techniques. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂, and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[Bis(carboxymethyl)amino]ethyl}(carboxymethyl)amino)acetic acid (EDTA), and 1.2-bis(o-aminophenoxy)ethane-N,N,N′,N′-tetraacetic acid (BAPTA)), reducing agents (e.g., dithiothreitol (DTT), β-mercaptoethanol (BME), and tris(2-carboxyethyl)phosphine (TCEP)), and labels (e.g., fluorophores, radiolabels, and spin labels). Buffers, cosolvents, salts, detergents/surfactants, chelators, reducing agents, and labels can be used at any suitable concentration, which can be readily determined by one of skill in the art. In general, buffers, cosolvents, salts, detergents/surfactants, chelators, reducing agents, and labels are included in reaction mixtures at concentrations ranging from about 1 μM to about 1 M. For example, a buffer, a cosolvent, a salt, a detergent/surfactant, a chelator, a reducing agent, or a label can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, the reaction mixture contains a glycan acceptor, one or more sugar donors, and a LegT, as well as one or more components selected from a buffer, a cosolvent, a salt, a detergent/surfactant, a chelator, and a reducing agent. In some embodiments, the reaction mixture consists essentially of an acceptor sugar, one or more sugar donors, and a LegT, as well as one or more components selected from a buffer, a cosolvent, a salt, a detergent/surfactant, a chelator, and a reducing agent.

In some embodiments, the reaction mixture is maintained at a temperature ranging from about 20° C. to about 30° C. in step (ii). In some embodiments, the reaction mixture is maintained in step (ii) for a period of time ranging from about 5 minutes to about 24 hours. In some embodiments, the period of time ranges from about 18 hours to about 22 hours.

The terms “about” and “around,” as used herein to modify a numerical value indicate a close range surrounding that explicit value. If “X” were the value, “about X” or “around X” would indicate a value from 0.9X to 1.1X. “About X” thus includes, for example, a value from 0.95X to 1.05X, or from 0.98X to 1.02X, or from 0.99X to 1.01X. Any reference to “about X” or “around X” specifically indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.07X, 1.08X, 1.09X, and 1.10X. Accordingly, “about X” and “around X” are intended to teach and provide written description support for a claim limitation of, e.g., “0.98X.”

Also provided herein are glycan products prepared from the methods as described herein.

Also provided herein are legionaminic acid transferase fusion proteins. In some embodiments, the LegT fusion protein comprises a polypeptide having at least 80% sequence identity to SEQ ID NO: 1, wherein the N-terminus of the polypeptide is fused to a maltose binding protein.

In some embodiments, the maltose binding protein comprises one or more valine substitutions.

Also provided herein are isolated nucleic acids encoding the LegT fusion proteins described herein. In some embodiments, the nucleic acid encodes for the LegT fusion protein having at least 80% sequence identity to SEQ ID NO:1, wherein the N-terminus of the polypeptide is fused to a maltose binding protein. In some embodiments, the nucleic acid encodes for the LegT fusion protein having at least 80% sequence identity to SEQ ID NO:1, wherein the N-terminus of the polypeptide is fused to a maltose binding protein, and the maltose binding protein comprises one or more valine substitutions.

Also provided are vectors containing the nucleic acids encoding the LegT fusion proteins described herein. In some embodiments, the vector contains the nucleic acid encoding the LegT fusion protein having at least 80% sequence identity to SEQ ID NO: 1, wherein the N-terminus of the polypeptide is fused to a maltose binding protein. In some embodiments, the vector contains the nucleic acid encoding for the LegT fusion protein having at least 80% sequence identity to SEQ ID NO:1, wherein the N-terminus of the polypeptide is fused to a maltose binding protein, and the maltose binding protein comprises one or more valine substitutions.

Also provided are host cells containing the nucleic acids encoding the LegT fusion proteins described herein. In some embodiments, the host cell contains the nucleic acid encoding the LegT fusion protein having at least 80% sequence identity to SEQ ID NO:1, wherein the N-terminus of the polypeptide is fused to a maltose binding protein. In some embodiments, the host cell contains the nucleic acid encoding for the LegT fusion protein having at least 80% sequence identity to SEQ ID NO:1, wherein the N-terminus of the polypeptide is fused to a maltose binding protein, and the maltose binding protein comprises one or more valine substitutions.

Also provided herein are vaccine compositions. The compositions contain one or more glycan products, including products prepared according to the method described herein, coupled to a carrier material. A vaccine composition according to the present disclosure can be used, for example, as a vaccine for a carbapenem-resistant A. baumannii strain (e.g., a clonal complex CC10 strain). In some embodiments, the vaccine is for an A. baumannii strain characterized by sequence type ST23. In some embodiments, the vaccine is for an A. baumannii strain comprising a KL8 gene cluster (an A. baumannii BAL 097 vaccine, an A. baumannii MDR-ZJ06 vaccine, an A. baumannii BJAB0715 vaccine, an A. baumannii XH858 vaccine, an A. baumannii 2004ZJAB5 vaccine, an A. baumannii 2004ZJAB6 vaccine, an A. baumannii AB1845 vaccine, an A. baumannii BAL vaccine, an A. baumannii BAL vaccine, an A. baumannii BAL206 vaccine, an A. baumannii 2011ZJAB1 vaccine, an A. baumannii XH683 vaccine, an A. baumannii 65 vaccine, or an A. baumannii XH769 vaccine) or an A. baumannii strain comprising a KL54 gene cluster (an A. baumannii RCH52 vaccine or an A. baumannii A457 vaccine).

Examples of carrier materials include, but are not limited to, carrier proteins such as a genetically modified cross-reacting material (CRM197) of diphtheria toxin, tetanus toxoid (TT), meningococcal outer membrane protein complex (OMPC), diphtheria toxoid (DD), and H. influenzae protein D (HiD). See, e.g., Pichichero (Human Vaccines & Immunotherapeutics 2013, 9(12): 2505-2523) and Berti et al. (Chem. Soc. Rev., 2018, 47, 9015-9025), which are incorporated herein by reference in their entireties.

Bacterial capsular saccharide products can be covalently bonded to proteins and other carrier materials using various chemistries for protein modification. A wide variety of such reagents are known in the art. Examples of such reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); imidoesters (amine reactive); isocyanates (hydroxyl reactive): vinyl sulfones (thiol, amine, and hydroxyl reactive); pyridyl disulfides (thiol reactive); and benzophenone derivatives (reactive via C—H bond insertion). Crosslinking reagents can react to form covalent bonds with functional groups in the bacterial capsular saccharide product (e.g., an aminopropyl group as described above) and in a protein or other carrier material (e.g., a primary amine, a thiol, a carboxylate, a hydroxyl group, or the like). Crosslinkers useful for attaching bacterial capsular saccharide products to proteins and other carrier materials include homobifunctional crosslinkers, which react with the same functional group in the bacterial capsular saccharide product and the carrier, as well as heterobifunctional crosslinkers, which react with functional groups in the bacterial capsular saccharide product and the carrier that differ from each other.

Examples of homobifunctional crosslinkers include, but are not limited to, amine-reactive homobifunctional crosslinkers (e.g., dimethyl adipimidate, dimethyl suberimidate, dimethyl pimilimidate, disuccinimidyl glutarate, disuccinimidyl suberate, bis(sulfosuccinimidyl) suberate, bis(diazo-benzidine), ethylene glycobis(succinimidyl-succinate), disuccinimidyl tartrate, disulfosuccinimidyl tartrate, glutaraldehyde, dithiobis(succinimidyl propionate), dithiobis-(sulfosuccinimidyl propionate), dimethyl 3,3′-dithiobispropionimidate, bis 2-(succinimidyl-oxycarbonyloxy)ethyl-sulfone, and the like) as well as thiol-reactive homobifunctional crosslinkers (e.g., bismaleidohexane, 1,4-bis-[3-(2-pyridyldithio)propionamido]butane, and the like). Examples of heterobifunctional crosslinkers include, but are not limited to, amine- and thiol-reactive crosslinkers (e.g., succinimidyl 4-(N-maleimidomethyl)-cyclohexane-1-carboxylate, m-maleimidobenzoyl-N-hydroxysuccinimide ester, succinimidyl-4-(p-maleimidophenyl)butyrate, N-(γ-maleimidobutyryloxy)succinimide ester, N-succinimidyl(4-iodoacetyl) aminobenzoate, 4-succinimidyl oxycarbonyl-α-(2-pyridyldithio)-toluene, sulfosuccinimidyl-6-α-methyl-α-(2-pyridyldithio)-toluamido-hexanoate, N-succinimidyl-3-(2-pyridyldithio) propionate, N-hydroxysuccinimidyl 2,3-dibromopropionate, and the like). Further reagents include but are not limited to those described in Hermanson, Bioconjugate Techniques 2nd Edition, Academic Press, 2008.

Vaccine compositions, or compositions thereof, can be administered to a subject by any of the routes normally used for administration of vaccines. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Appropriate pharmaceutically acceptable carriers can be selected based on facts including, but not limited to, the particular composition being administered, as well as by the particular method used to administer the composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In some embodiments, the vaccine composition is sufficiently immunogenic as a vaccine for effective immunization without administration of an adjuvant. In some embodiments, immunogenicity of a composition is enhanced by including an adjuvant. Any adjuvant may be used in conjunction with the vaccine composition. A large number of adjuvants are known; see, e.g., Allison, 1998, Dev. Biol. Stand., 92:3-11, Unkeless et al., 1998, Annu. Rev. Immunol., 6:251-281, and Phillips et al., 1992, Vaccine, 10:151-158. Exemplary adjuvants include, but are not limited to, cytokines, gel-type adjuvants (e.g., aluminum hydroxide, aluminum phosphate, calcium phosphate, etc.), microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.), oil-emulsion and emulsifier-based adjuvants (e.g., Freund's Adjuvant, MF59 [Novartis], SAF, etc.), particulate adjuvants (e.g., liposomes, biodegradable microspheres, saponins, etc.), synthetic adjuvants (e.g., nonionic block copolymers, muramyl peptide analogues, polyphosphazene, synthetic polynucleotides, etc.) and/or combinations thereof.

### EXAMPLES

### Example 1. Cloning and Purification of LegT

Without being limited by theory, AbGtr18 (GenBank accession number AQQ74347.1) from A. baumannii K8 may be responsible for the transfer of the Leg5Ac7R residue to α-D-GalNAc via an α2-6 linkage, where R indicates (R)-3-hydroxybutanoyl or acetyl in the ratio ˜2.5:1. This 323-residue protein belongs to glycosyltransferase (GT) family 52 (GT52) in the Carbohydrate-Active enZYmes (CAZy) database, which also contains α2-3-sialyltransferases and α-glucosyltransferases. Further sequence alignment revealed that AbGtr18 shares 74.5%, 33.1%, 34.7% and 32.6% identity with AbGtr109 (GenBank accession number AWJ68081.1) from A. baumannii K54, AbGtr56_{K27} (GenBank accession number ALL34863.1) from A. baumannii K27, AbGtr56K44(GenBank accession number QGW59139.1) from A. baumannii K44, and AbGtr16 (GenBank accession number AGM37788.1) from A. baumannii K6. Leg and derivatives also present in the CPS repeating units of A. baumannii K54, A. baumannii K27 and A. baumannii K44. AbGtr109, Gtr56_{K27} and Gtr56_{K44} were assigned as corresponding Leg-glycosyltransferases. Among them, Gtr56_{K27} and Gtr56_{K44} were found to be 90% identical to each other. The repeating unit of the K6 CPS recovered from strain A. baumannii K6 was found to contain Pse5Ac7Ac. Pse5Ac7Ac and Leg5Ac7R are both non-2-ulosonic acids, and AbGtr16 was predicted to be an inverting glycosyltransferase adding Pse5Ac7Ac. Therefore, AbGtr18 (GenBank accession number AQQ74347) was chosen for further characterization herein.

Bacterial strains, plasmids, and materials. E. coli DH5a chemically competent cells were from Invitrogen (Carlsbad, CA). Bio-Scale Mini Nuvia IMAC Cartridges and Bio-Scale™ Mini Bio-Gel® P-6 Desalting Cartridges were from Bio-Rad (Hercules, CA, USA). AccuPrep® PCR/Gel purification kits were from BIONEER Corporation. GeneJET plasmid spin kits, 1 kb DNA ladder and pre-stained protein ladder were from Fisher Scientific (Tustin, CA, USA). Phusion® HF DNA polymerase was from New England Biolabs Inc. (Beverly, MA, USA). pMAL-c4X was a gift from Paul Riggs (Addgene plasmid #75288). Synthetic genes encoding His₆-AbGtr18, His₆-AbFn1A (SEQ ID NO:9), and His₆-AbFn1B (SEQ ID NO:11) were cloned into expression vector pET28a between BamHI and XhoI sites by Twist Bioscience (South San Francisco, CA, USA). 4-Nitrophenyl-α-D-galactopyranoside (GalαpNP) and 4-nitrophenyl β-D-galactopyranoside (GalβpNP) are commercially available from Fisher Scientific (Tustin, CA, USA). GalNAcα2AA, GalNAcαProNHCbz, GalNAcβProNHCbz, GlcNAcαProNHCbz, GlcNAcβProNHCbz, LacβProNHCbz, LacNAcβProNHCbz, Galβ1-3GalNAcαProNHCbz, Galβ1-3GalNAcβProNHCbz, Galβ1-3GlcNAcαProNHCbz, Galβ1-3GlcNAcβProNHCbz were prepared.

Gene cloning, protein overexpression and purification. The synthetic gene encoding the full-length AbGtr18 was used as the template to amplify MBP-AbGtr18-His₆. The primers used for cloning are listed in Table 1 (corresponding to SEQ ID NO: 14 and SEQ ID NO:15). PCRs were performed in 50 μL reaction mixture containing 5 ng of recombinant plasmid, 1 μM each of forward and reverse primers, 5 μL of 10×Phusion® HF buffer, 1 mM dNTP mixture, and 5 units (1 μL) of Phusion® HF DNA polymerase. The reaction mixtures were subjected to 35 cycles of amplifications with an annealing temperature at 55° C. The resulting PCR products were digested with the corresponding restriction enzymes introduced in the primers, purified, and ligated with predigested pMal-c4× vector containing a high-binding mutant (A313V) of maltose-binding protein (MBP). Recombinant plasmids bearing the target genes were transformed to chemically competent E. coli DH5α cells. Positive plasmids were sequenced and subsequently transformed into homemade BL21 (DE3) chemically competent cells. Selected clones were grown for protein expression.

TABLE 1
Primers used for cloning transferases.

| | Oligo- | |
|---|---|---|
| | nucleo- | |
| Primers | tides^{[a]} | |
| MBP- | Forward | GTTTTTGAATTCATGGAACATCGTCAAAAGAG |
| AbGtr18- | (SEQ ID | EcoRI |
| His6 | NO: 14) | GTTTTTTGTCGACTCAATGATGATGATGATGA |
| | Reverse | TGGATGTCCTTGAACTTCAGAA |
| | (SEQ ID | SalI |
| | NO: 15) | |

^{[a]}Restriction sites are italicized and underlined.

Plasmid-bearing E. coli cells were cultured in 1 L 2YT medium (16 g/L tryptone, 10 g/L yeast extract, and 5 g/L NaCl) supplemented with 50 g/mL kanamycin or 100 μg/mL ampicillin at 37° C. with shaking. Generally, overexpression of the target protein was achieved by inducing the E. coli culture with 0.1 mM of isopropyl-1-thio-β-D-galactopyranoside (IPTG) at OD_{600 nm}=0.6-1.0 and incubating at 20° C. for 20 h with vigorous shaking at 220 rpm in a C25KC incubator shaker (instrument commercially available from New Brunswick Scientific, Edison, NJ). Cells were collected by centrifugation at 5000×g, 4° C. for 30 minutes. The cell precipitation was re-suspended in Tris-HCl buffer (100 mM, pH 7.5) plus 0.1% TRITON X-100 (t-octylphenoxypolyethoxyethanol) (commercially available from Sigma Aldrich, St. Louis, MO), and then lysed by homogenizer. Cell debris was removed by centrifugation at 9,016 g and 4° C. for 30 minutes, and the enzymes were purified from the supernatant by BIO-SCALE MINI NUVIA IMAC cartridge following the manufacturer's instructions. Eluted fractions were pooled and dialyzed against Tris-HCl buffer (20 mM, pH 7.5 plus 0.2 M NaCl). The expression of the recombinant proteins was examined by SDS-polyacrylamide gel electrophoresis (SDS-PAGE) performed in 12% Tris-glycine gels, and the protein concentration was determined by NANODROP LITE spectrophotometer from Fisher Scientific (Tustin, CA, USA).

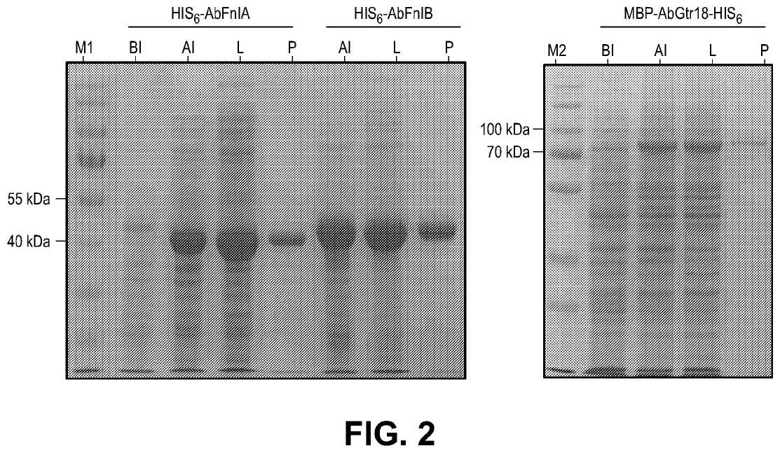

Results. Recombinant AbFn1A (SEQ ID NO: 10) and AbFn1B (SEQ ID NO:12) were cloned into pET28a(+) vector as N-His₆-tagged proteins and expressed in BL21 (DE3). Soluble recombinant proteins were readily purified by nickel-nitrilotriacetic acid (Ni2+-NTA) affinity chromatography with yields of 105 and 157 mg per liter 2YT medium with expected molecular weights of about 42.4 kDa and 45.2 kDa for AbFn1A and AbFn1B (FIG. 2), respectively. Recombinant AbFn1C, AbGtr18, AbGtr19 and AbGtr20 were also cloned into pET28a(+) vector as N-terminal His₆-tagged proteins and expressed in BL21 (DE3). However, only around 8 mg of His₆-AbGtr18 was obtained from 1 liter of culture and no significant expression of soluble proteins was observed for others. Therefore, AbFn1C, AbGtr18, AbGtr19 and AbGtr20 were sub-cloned into pMAL-c4X (Plasmid #75288) as C-His₆-tagged proteins fusing to a high-binding mutant (A313V) maltose-binding protein (MBP) in the N terminus and expressed under similar conditions. After Ni2+-NTA affinity chromatography, around 75 mg, 45 mg, 45 mg, and 20 mg of MBP-AbFn1C-His₆, MBP-AbGtr18-His₆, MBP-AbGtr19-His₆ and MBP-AbGtr20-His₆ were obtained from 1 L culture with expected molecular weights of about 85.9 kDa, 82.5 kDa, 86.6 kDa and 84.1 kDa, respectively.

### Example 2. Characterization of Legionaminic Acid Transferase AbGtr18

pH profile of His₆-AbGtr18. Enzymatic assays (10 μL total reaction volume) were performed in duplicate at 35° C. for 30 minutes in a buffer (100 mM) with a pH in the range of 3.0-10.0, MgCl₂ (10 mM), donor CMP-Leg5,7Ac₂ (1 mM), GalNAcα2AA (1 mM), and His₆-AbGtr18 (0.64 μM). Buffers used were: citric acid-sodium citrate, pH 3.0-6.5; Na₂HPO₄-citric acid, pH 6.0-8.0; Tris-HCl, pH 7.0-9.0; and glycine-NaOH, pH 8.5-11.0, respectively. Reactions were stopped by adding 40 μL methanol. Samples were centrifuged, and the supernatants were analyzed at 254 nm by an AGILENT ultra-high performance liquid chromatography (UHPLC) system equipped with a membrane on-line degasser, a temperature control unit (set at 30° C.), and a diode array detector using ECLIPSE PLUS C18 RRHD column (2.1×50 mm I.D., 1.8 μm particle size; AGILENT). Mobile phase A was 0.1% trifluoroacetic acid (TFA) in water, and mobile phase B was acetonitrile. The system was pre-equilibrated with a running mobile phase composed of mobile phase A and mobile phase B (95/5, v/v) at a flow rate of 0.25 mL/min. After injection of the sample, compound separation was carried out with two-phase gradient elution steps (starting at 90% A+10% B at 0 min to 50% A+50% B at 4 minutes, then back to 90% A+10% B at 5 min with the run stopped at 6 minutes).

Temperature profile of His₆-AbGtr18. Enzymatic assays were carried out in duplicate for 30 minutes in a total volume of 10 μL in citric acid-sodium citrate buffer containing 1 mM CMP-Leg5,7Ac₂, 1 mM GalNAcα2AA, His₆-AbGtr18 (0.64 μM), and 10 mM of MgCl₂ at different temperatures: 15° C., 20° C., 25° C., 30° C., 35° C., 37° C., 40° C., 45° C., 50° C., 55° C., and 60° C., respectively. The reactions were quenched by adding 40 μL methanol. Samples were centrifuged, and then analyzed by UHPLC as described above for pH profile studies.

Effects of metal ions, EDTA, and a reducing reagent DTT on the activity of His₆-AbGtr18. Enzymatic assays were carried out in duplicate at 40° C. for 30 minutes in a total volume of 10 μL in citric acid-sodium citrate buffer (0.1 μM, pH 6.0) containing 1 mM CMP-Leg5,7Ac₂, 1 mM GalNAcα2AA, His₆-AbGtr18 (0.64 μM), and 10 mM of CaCl₂), CuSO₄, MgCl₂, MnCl₂, NiSO₄, ZnCl₂, ethylenediaminetetraacetic acid (EDTA), or dithiothreitol (DTT). Reactions without metal ions, EDTA, or DTT were used as controls. The reactions were quenched by adding 40 μL methanol. Samples were centrifuged, and then analyzed by UHPLC as described above for pH profile studies.

Time-course studies of His₆-AbGtr18. Enzymatic assays were carried out in duplicate at 30° C. and 40° C., respectively, in a total volume of 110 μL in citric acid-sodium citrate buffer (0.1 μM, pH 6.0) containing 1.5 mM CMP-Leg5,7Ac₂, 1 mM GalNAcα2AA, His₆-AbGtr18 (0.52 μM). 10 μL reaction mixture was taken out after reacting for 5 minutes (min), 10 minutes, 20 minutes, 40 minutes, 1 hour (h), 2 hours, 4 hours, 7 hours, 10 hours and 24 hours and quenched by adding 20 μL methanol. Samples were centrifuged, and then analyzed by UHPLC as described above for pH profile studies.

Thermostability studies of His₆-AbGtr18. His₆-AbGtr18 was incubated for 1 hour at different temperatures: 10° C., 15° C., 20° C., 25° C., 30° C., 35° C., 37° C., 40° C., 45° C., 50° C., 55° C., and 60° C., respectively. Incubated His₆-AbGtr18 (1.11 μM) was then added into 10 μL reaction mixture in citric acid-sodium citrate buffer (0.1 μM, pH 6.0) containing 1.5 mM CMP-Leg5,7Ac₂ and 1 mM GalNAcαProNHCbz to start enzymatic assays in duplicate at 37° C. for 15 min. Samples were centrifuged, and the supernatants were analyzed at 210 nm by an AGILENT ultra-high performance liquid chromatography (UHPLC) system equipped with a membrane on-line degasser, a temperature control unit (set at 30° C.), and a diode array detector using AGILENT ZORBAX RRHD Bonus-RP column (2.1×150 mm I.D., 1.8 m particle size; AGILENT). Mobile phase A was 0.1% trifluoroacetic acid (TFA) in water, and mobile phase B was acetonitrile. The system was pre-equilibrated with a running mobile phase composed of mobile phase A and mobile phase B (95/5, v/v) at a flow rate of 0.3 mL/min. After injection of the sample, compound separation was carried out with two-phase gradient elution steps (starting at 95% A+5% B with a flow rate of 0.3 m/min at 0 min to 44% A+56% B with a flow rate of 0.1 m/min at 7 min, then back to 90% A+10% B with a flow rate of 0.3 mL/min at 8 min with the run stopped at 10.5 min).

Acceptor substrate specificity studies of His₆-AbGtr18. Enzymatic assays were carried out in duplicate at 30° C. in a total volume of 10 μL in citric acid-sodium citrate buffer (0.1 μM, pH 6.0) containing 1.5 mM CMP-Leg5,7Ac₂ and 1 mM acceptor. GalNAcα2AA, GalNAcαProNHCbz, GalNAcβProNHCbz, GlcNAcαProNHCbz, GlcNAcβProNHCbz, LacβProNHCbz, LacNAcβProNHCbz, Galβ1-3GalNAcαProNHCbz, Galβ1-3GalNAcβProNHCbz, Galβ1-3GlcNAcαProNHCbz, Galβ1-3GlcNAcβProNHCbz, GalαpNP and GalβpNP were used as acceptors. 0.55 M and 5.5 M of His₆-AbGtr18 were added to start the reaction for 15 minutes and 20 hours, respectively. Samples were centrifuged, and the supernatants were analyzed by UHPLC similarly to that described above for thermostability studies.

Donor specificity of MBP-AbGtr18-His₆. CMP-Leg5,7Ac₂, CMP-sialic acids, and derivatives were generated in situ from Leg5,7Ac₂, Neu5Ac, and their derivatives or precursors. All reactions were carried out in duplicate. For reactions with Leg5,7Ac₂ or a derivative as the starting material, each reaction mixture had a total volume of 10 μL and contained Tris-HCl buffer (pH 7.5, 100 mM), Leg5,7Ac₂ or a derivative (5 mM), cytidine 5′-triphosphate (CTP) (7.5 mM), MgCl₂ (20 mM), and L. pneumophila CMP-5,7-di-N-acetyllegionaminic acid synthetase (LpCLS) (3.6 g) or NmCSS (5 g). For reactions with a sialic acid precursor as the starting material, ManNAc or its derivative (5 mM) and sodium pyruvate (25 mM) were used to replace the sialic acid or its derivative (5 mM), and PmAldolase (20 μg) was added while other components remained the same. The reactions were allowed to proceed at 30° C. for 90 minutes. At that point, 2.4 μL of each reaction mixture was pipetted out for setting up the transferase assays as described below. The reaction of the remaining mixture was stopped by adding 7.6 μL of cold methanol to each tube followed by incubation on ice for 30 minutes and centrifugation at 13,800 g for 10 minutes. The supernatant was used for the UHPLC assays and HRMS analysis. Chromatographic separation and detection were achieved with an INFINITY 1290 II HPLC (UHPLC) equipped with 1260 INFINITY II Diode Array Detector WR (monitored at 254 nm, Agilent Technologies, CA) with a CARBOPAC PA100 (4×250 mm, Thermo Scientific, CA) and a gradient flow (0.7 mL/min) of a mixed solvent (0% to 30% 1 M NaCl, and 100% to 70% of water) in a duration of 19 minutes. All transferase donor substrate specificity assays were performed in duplicate in a reaction mixture (10 μL) containing Tris-HCl buffer (pH 7.5, 100 mM), a reaction mixture of in-situ-generated CMP-Leg5,7Ac₂ or derivative (2.4 μL), GalNAcαProNHCbz (1 mM), MgCl₂ (20 mM), and MBP-AbGtr18-His₆ (2.1 M). The reaction mixtures were allowed to proceed at 30° C. for 30 minutes and 20 hours, respectively. The reactions were stopped by adding 10 μL of cold methanol to each reaction mixture, followed by incubation on ice for 30 minutes and centrifugation at 13,800 g for 10 minutes. The supernatant was used for the UHPLC assays and HRMS analysis. UHPLC separation and detection were carried out similarly to that described above for thermostability studies.

Time-course study of MBP-AbGtr18-His₆ using in situ generated CMP-Leg5,7Ac₂ and CMP-Neu5Ac. CMP-Leg5,7Ac₂ and CMP-Neu5Ac were generated as described above. Enzyme assays were performed at 30° C. in duplicate in a total volume of 100 μL in Tris-HCl buffer (pH 7.5, 100 mM) containing in-situ-generated CMP-Leg5,7Ac₂ or CMP-Neu5Ac (1.35 mM), GalNAcαProNHCbz (1 mM), MBP-AbGtr18-His₆ (0.60 μM). A 10 μL aliquot of the reaction mixture was taken out after reacting for 5 minutes, 10 minutes, 20 minutes, 40 minutes, 1 hour, 2 hours, 3 hours, 5 hours, and 20 hours and quenched by adding 20 μL pre-chilled methanol. Samples were centrifuged, and then analyzed by UHPLC as described above for thermostability studies.

Glycosyltransferase activity assays for MBP-AbGtr18-His₆, His₆-Pd2,6ST(16-497), Psp2,6ST(15-501)-His₆ and its A366G mutant. CMP-Leg5,7Ac₂ and CMP-Neu5Ac were generated as described above. Enzyme assays were performed in duplicate in a total volume of 20 μL in Tris-HCl buffer (pH 7.5, 100 mM) in-situ-generated CMP-Leg5,7Ac₂ or CMP-Neu5Ac (1.35 mM), GalNAcαProNHCbz or LacβProNHCbz (1 mM), and a certain amount of MBP-AbGtr18-His₆ or sialyltransferases. The reaction mixtures were allowed to proceed at 30° C. for a certain amount of time, and then 10 μL of the reaction mixtures were taken out and stopped by adding 10 μL of cold methanol. The reaction of the remaining mixture was allowed to proceed at 30° C. for 20 hours and then stopped by adding 10 μL of cold methanol to each reaction mixture, followed by incubation on ice for 30 minutes and centrifugation at 11,337 g for 10 minutes. Samples were centrifuged, and then analyzed by UHPLC described above for thermostability studies.

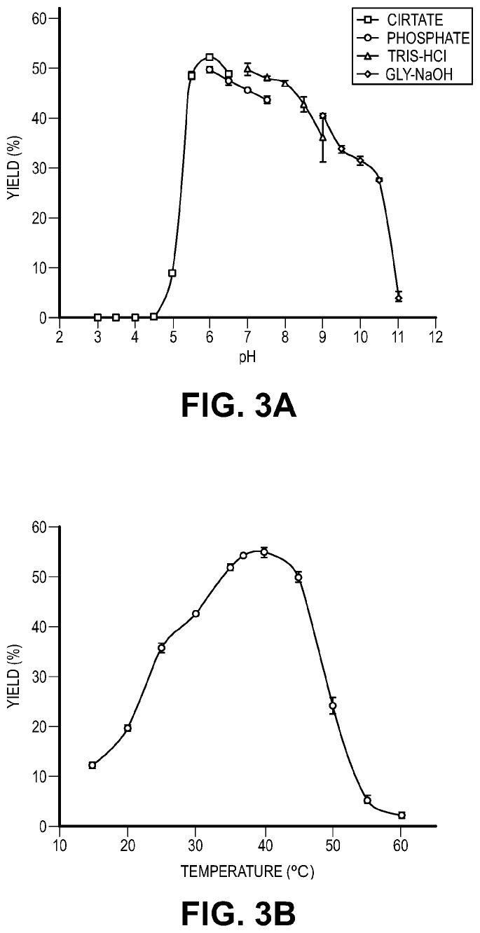

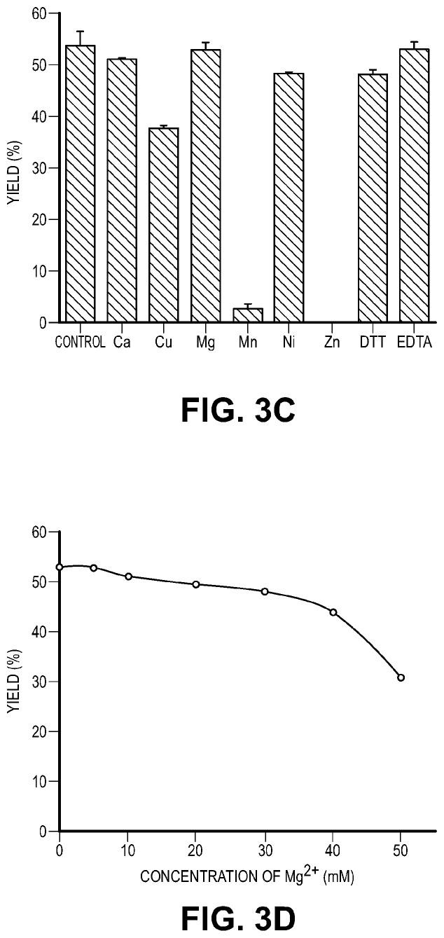

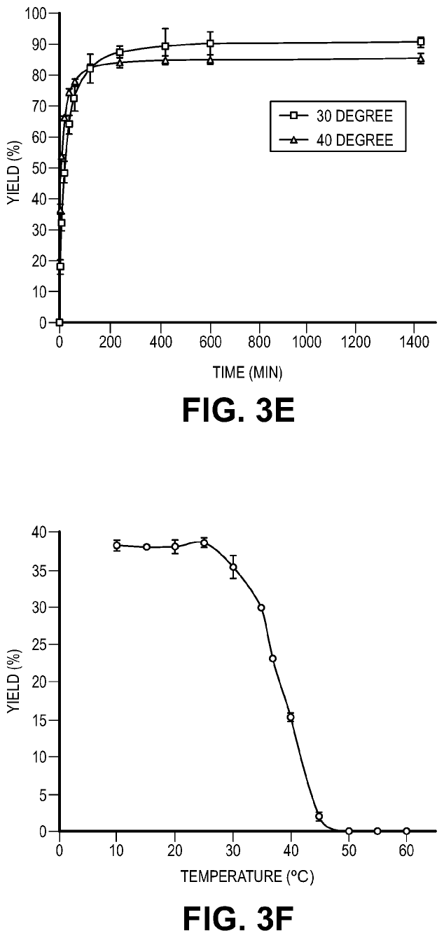

His₆-AbGtr18 shows broad pH preference and the optimal activity was at pH 6.0 in sodium citrate buffer for CMP-Leg5,7Ac₂ (FIG. 3A). It was shown to have optimal activities at 40° C. (FIG. 3B). As shown in FIG. 3C, a divalent metal cation was not required for the catalytic activity of AbGtr18, as 10 mM of EDTA had no effect. Ca²⁺ and DTT had no significant effect on the activity of AbGtr18. The addition of Mn²⁺ or Zn²⁺ almost abolished its activity completely. The presence of 10 mM Cu²⁺ decreased the reaction yields of His₆-AbGtr18 on CMP-Leg5,7Ac2. The addition of Mg²⁺ had no significant effect for CMP-Leg5,7Ac2. Further increasing the concentration of Mg²⁺ up to 50 mM can decrease the activity of His₆-AbGtr18 on CMP-Leg5,7Ac₂ (FIG. 3D). As indicated by the time-course studies undertaken respectively at 30° C. and 40° C., as shown in FIG. 3E, the latter temperature showed superior catalytic performance over the former one within the first 1 hour. However, as time went by, the overall yield of reaction under 30° C. became higher than that under 40° C., indicating 30° C. to be suitable for future preparative synthesis. The following thermostability study suggested that AbGtr18 was stable when the incubation temperature was no higher than 30° C. with at least 90% activity retained. When compared with incubation at 20° C., this enzyme retained about 77%, 60%, 40% and 5% activities, respectively, after incubation at 35° C., 37° C., 40° C. and 45° C. for 1 hour. AbGtr18 was totally denatured when temperature was higher than 45° C.

### Example 3. Compound Synthesis

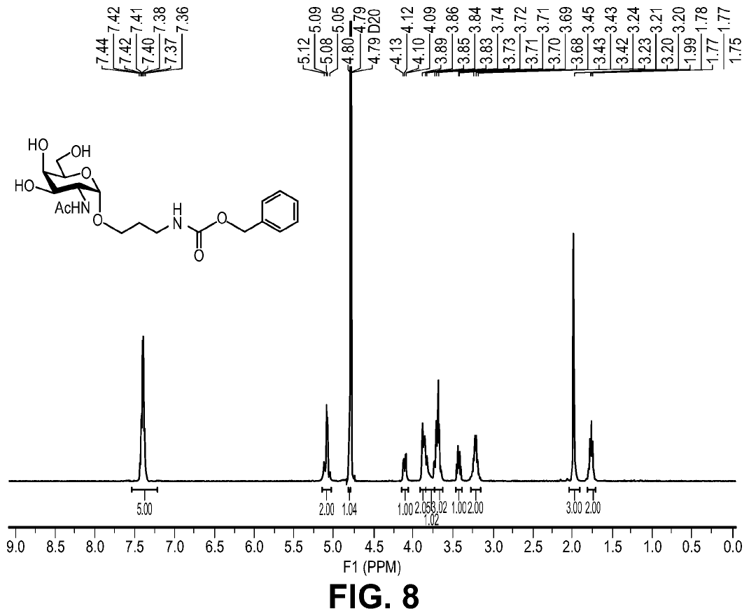

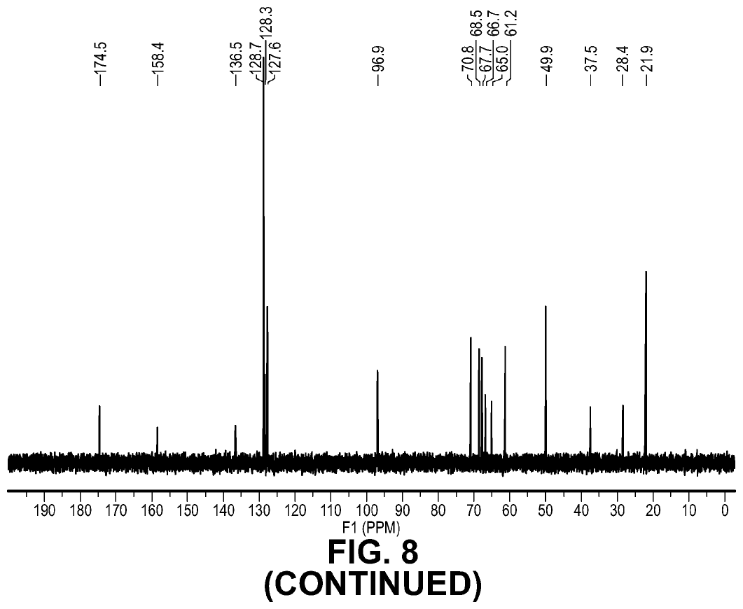

Synthesis of GalNAcαProNHCbz. To a solution of D-GalNAc (1.0 g, 4.52 mmol) in 3-chloropropanol (20 mL) was added acetyl chloride (322 μL, 4.52 mmol) dropwise at 0° C. The reaction mixture was heated at 70° C. for 3 hours. The solution was concentrated and the residue was purified by silica gel chromatography to yield GalNAcαProCl (996 mg, 74%). The obtained GalNAcαProCl (996 mg, 3.35 mmol) was dissolved in DMF (10 mL), and NaN₃ (2.17 g, 33.45 mmol) was added. The reaction mixture was heated at 70° C. for 6 hours. The reaction mixture was then concentrated and the residue was purified using a silica gel column with EtOAc:MeOH:H₂O=10:2:0.1 as the mobile phase. The fractions containing pure products were combined, concentrated in a rotavap, and dried in vacuo to yield GalNAcαProN₃ (998 mg, 3.28 mmol, 98%) as a white solid. Next, catalytic hydrogenation of azide to amine was done by redissolving in anhydrous MeOH (10 mL), and adding 10% Pd/C (100 mg), followed by hydrogenation using hydrogen ballon. After the completion of the reaction, palladium was removed by filtration. The solvent was removed in vacuo. The obtained compound was used directly for the next reaction without any further purification. The compound (3.28 mmol) was dissolved in saturated sodium carbonate water (6 mL) and benzyl chloroformate (CbzCl, 1.25 mL, 9.81 mmol) in acetonitrile (6 mL) was added at 0° C. After completion of the reaction, the solvent was removed. The mixture was purified using a C18 column (gradient solvent of CH₃CN in H₂O was used for elution) to produce the GalNAcαProNHCbz as a white solid (1.04 g, 77%) (Scheme 1). ¹H NMR (400 MHz, D₂O) δ 7.44-7.36 (m, 5H), 5.09 (dd, J=12.8, 4.3 Hz, 2H), 4.80 (d, J=2.9 Hz, 1H), 4.11 (dd, J=10.9, 3.7 Hz, 1H), 3.89-3.83 (m, 3H), 3.74-3.65 (m, 3H), 3.43 (dt, J=10.1, 5.8 Hz, 1H), 3.22 (q, J=6.2 Hz, 2H), 1.99 (s, 3H), 1.76 (dd, J=12.6, 6.7 Hz, 2H); ¹³C NMR (100 MHz, D₂O) δ 174.5, 158.4, 136.5, 128.7, 128.7, 128.3, 127.6, 127.6, 96.9, 70.8, 68.5, 67.7, 66.7, 65.0, 61.2, 49.9, 37.5, 28.4, 21.9; HRMS (ESI-Orbitrap) m/z: [M+Na]⁺ calculated for C₁₉H₂₈N₂O₈Na 435.1743; found 435.1731. The ¹H and ¹³C NMR spectra are shown in FIG. 8.

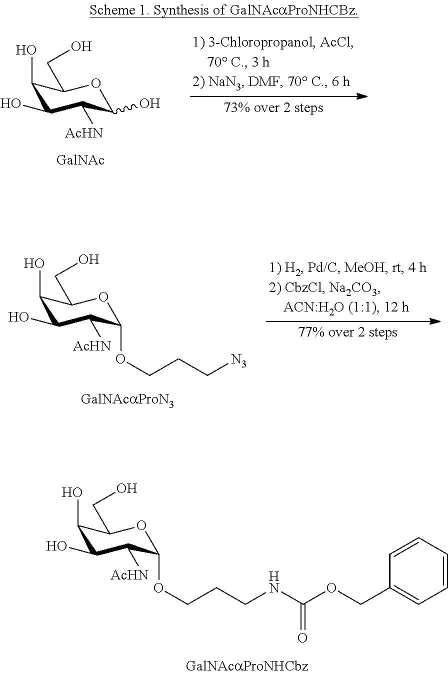

One-pot two-enzyme (OP2E) preparative-scale synthesis of disaccharide Neu5Acα2-6GalNAcαProNHCbz. MBP-AbGtr18-His₆ was used together with a recombinant Neisseria meningitidis CMP-sialic acid synthetase (NmCSS) in a one-pot two-enzyme (OP2E) system (Scheme 2) for the synthesis of Neu5Acα2-6GalNAcαProNHCbz from GalNAcαProNHCbz with a 94% yield.

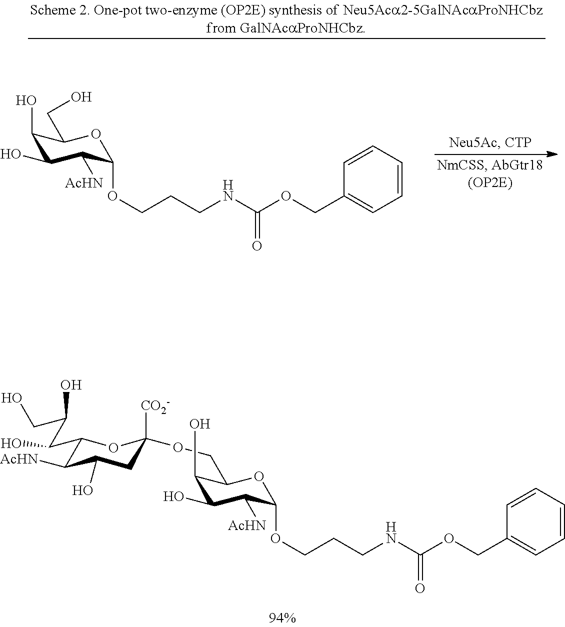

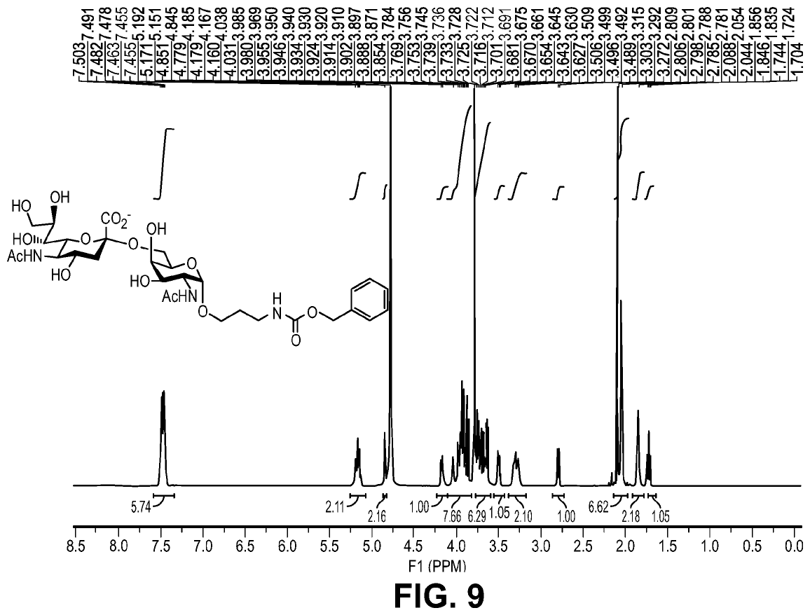

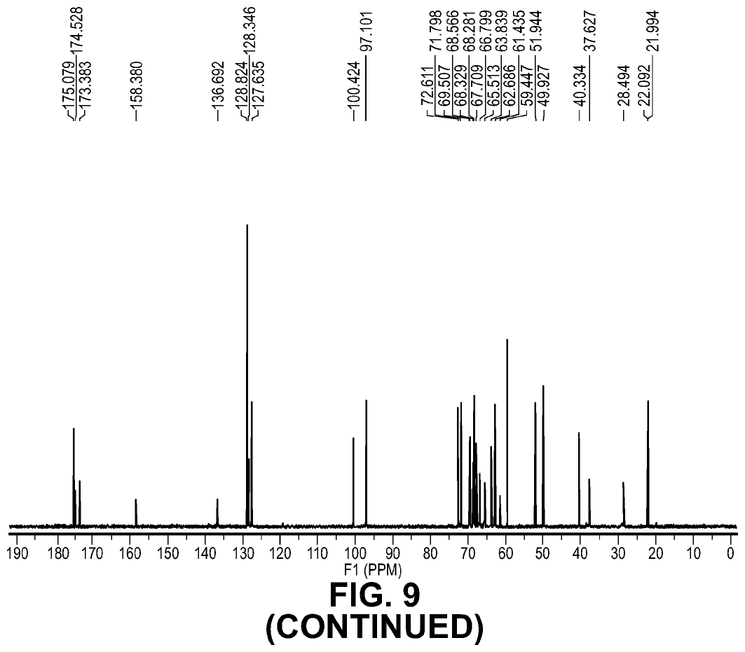

GalNAcαProNHCbz (100 mg, 0.24 mmol), Neu5Ac (97 mg, 0.31 mmol), and CTP (191 mg, 0.36 mmol) were incubated at 30° C. in a Tris-HCl buffer (100 mM, pH 8.5) containing MgCl₂ (20 mM), NmCSS (1.5 mg), and MBP-AbGtr18-His₆ (4 mg). The reaction with total 20 mL volume was incubated at 30° C. for 16 h with agitation at 180 rpm. The product formation was monitored by TLC with EtOAc:MeOH:H₂O=5:2.4:1 (by volume) as the developing solvent and by HRMS. After the reaction was completed, the reaction was incubated in a boiling water bath for 5 minutes to denature the enzymes, then centrifuged at 9,016 g for 30 minutes at 4° C. The supernatant was concentrated and purified by a C18 cartridge (51 g, 50 μm, 120 Å, Yamazen) on a CombiFlash system to obtain the pure product as a white powder (165 mg, 94%). ¹H NMR (600 MHz, D₂O) δ 7.51-7.43 (m, 5H), 5.19-5.13 (m, 2H), 4.85 (d, J=4.0 Hz, 1H), 4.17 (dd, J=11.4, 3.6 Hz, 1H), 4.05-3.48 (m, 12H), 3.38-3.17 (m, 2H), 2.79 (dd, J=12.2, 4.8 Hz, 1H), 2.09 (m, 3H), 2.05 (m, 3H), 1.87-1.82 (m, 2H), 1.72 (t, J=12.0 Hz, 1H). ¹³C NMR (150 MHz, D₂O) δ 175.08, 174.53, 173.38, 158.38, 136.69, 128.82, 128.35, 127.63, 100.42, 97.10, 72.61, 71.80, 69.51, 68.57, 68.33, 68.28, 67.71, 66.78, 65.51, 63.84, 62.69, 61.43, 59.45, 51.94, 49.93, 40.33, 37.63, 28.49, 22.09, 21.99. HRMS (ESI-Orbitrap) m/z: [M−H]⁻ calculated for C₃₀H₄₄N₃O₁₆ 702.2727; found 702.2735. The ¹H and ¹³C NMR spectra are shown in FIG. 9.

One-pot two-enzyme (OP2E) synthesis of disaccharide Neu5Acα2-6GalNAc. Similarily, synthesis of Neu5Acα2-6GalNAc from D-GalNAc and Neu5Ac using the OP2E containing MBP-AbGtr18-His₆ and NmCSS was achieved with a 95% yield (Scheme 3).

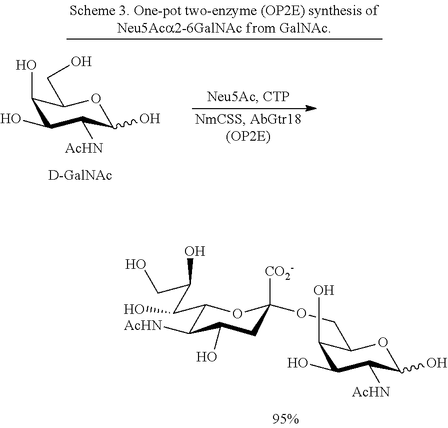

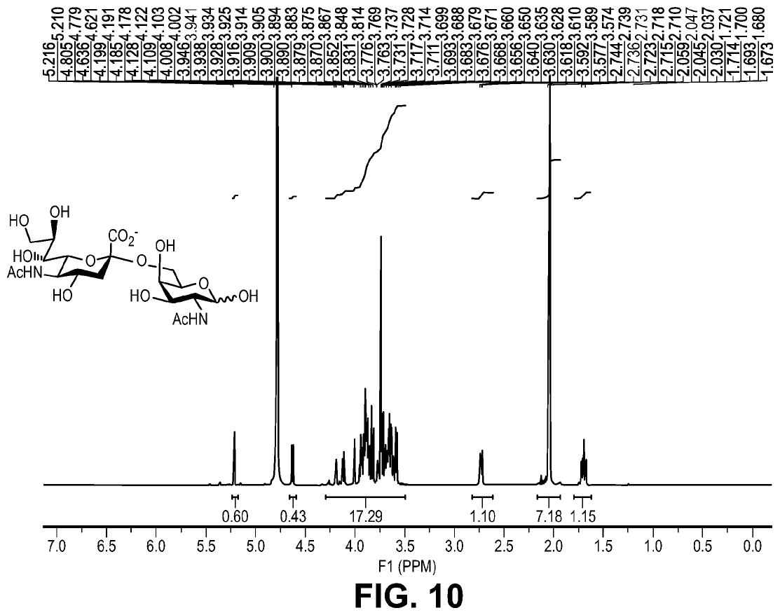

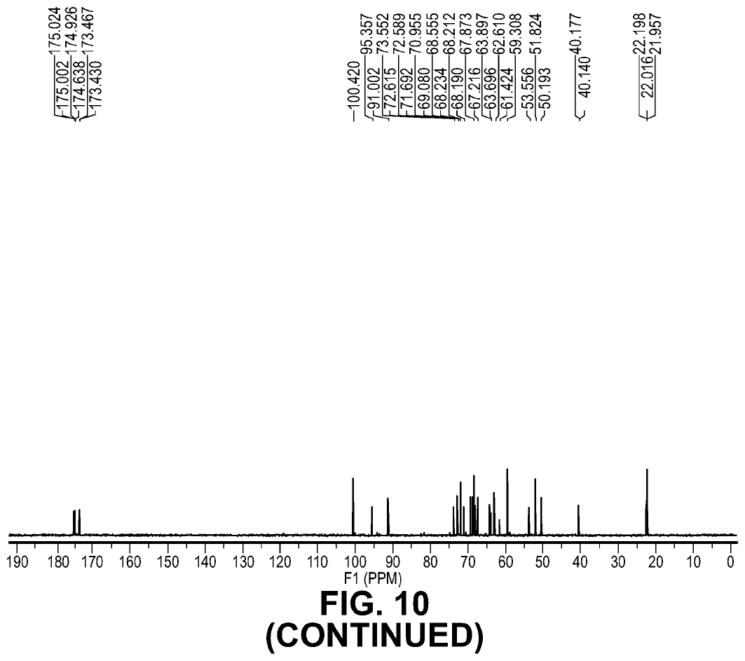

D-GalNAc (100 mg, 0.45 mmol), Neu5Ac (182 mg, 0.58 mmol), and CTP (357 mg, 0.68 mmol) were incubated at 30° C. in a Tris-HCl buffer (100 mM, pH 8.5) containing MgCl₂ (20 mM), NmCSS (2.0 mg), and MBP-AbGtr18-His₆ (5 mg). The reaction with total 20 mL volume was incubated at 30° C. for 18 hours with agitation at 180 rpm. The product formation was monitored by HRMS. After the reaction was completed, 5 mg of alkaline phosphatase (from bovine) was added and the reaction was incubated at 30° C. for 15 hours with agitation at 180 rpm. The reaction was incubated in a boiling water bath for 5 minutes to denature the enzymes, then centrifuged at 9,016 g for 30 minutes at 4° C. The supernatant was concentrated and purified by a BIO-GEL P-2 column to obtain the pure product as a white powder (230 mg, 95%). ¹H NMR (600 MHz, D₂O) δ 5.21 (d, J=3.6 Hz, 0.6H), 4.63 (d, J=9.0 Hz, 0.4H), 4.29-3.49 (m, 13H), 2.75-2.71 (m, 1H), 2.05 (s, 3H), 2.04 (s, 3H), 1.73-1.67 (m, 1H). ¹³C NMR (150 MHz, D₂O) δ 175.02, 175.00, 174.93, 174.64, 173.47, 173.43, 100.42, 95.36, 91.00, 73.55, 72.61, 72.59, 71.69, 70.96, 69.08, 68.56, 68.23, 68.21, 68.19, 67.87, 67.22, 63.90, 63.70, 62.61, 61.42, 59.31, 53.56, 51.82, 50.19, 40.18, 40.14, 22.20, 22.02, 21.95. HRMS (ESI-Orbitrap) m/z: [M−H]⁻ calculated for C₁₉H₃₁N₂O₁₄ 511.1781; found 511.1770. The ¹H and ¹³C NMR spectra are shown in FIG. 10.

One-pot two-enzyme (OP2E) and one-pot three-enzyme (OP3E) syntheses of disaccharide Leg5,7Ac₂α2-6GalNAcαProNHCbz. Synthesis of Leg5,7Ac₂α2-6GalNAcαProNHCbz from GalNAcαProNHCbz was achieved using a OP2E system containing Legionella penumophila CMP-Leg5,7Ac₂ synthetase (LpCLS) and MBP-AbGtr18-His₆ with a 99% yield using Leg5,7Ac₂ as a donor precursor (Scheme 4). It was also achived from a OP3E system containing Pasteurella multocida sialic acid aldolase (PmAldolase), LpCLS, and MBP-AbGtr18-His₆ with a 94% yield using 6deoxyMan2,4diNAc as a donor precursor (Scheme 4).

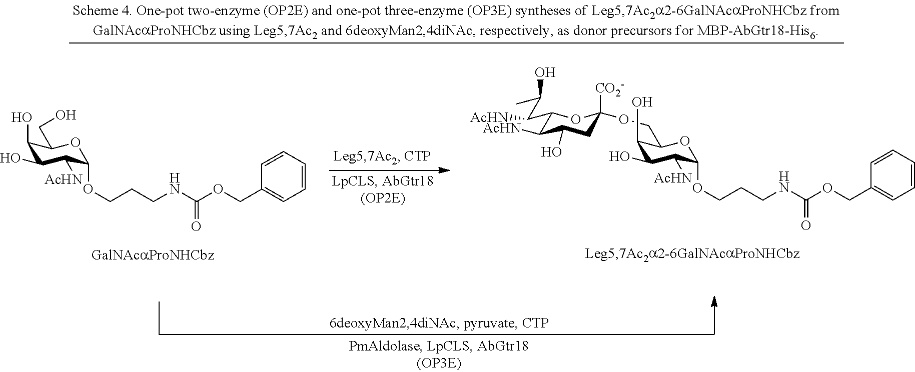

OP2E synthesis of disaccharide Leg5,7Ac₂α2-6GalNAcαProNHCbz. Each reaction was carried out at 30° C. for 5 hours in an aqueous solution (10 μL) containing Leg5,7diNAc (7.5 mM), CTP (10 mM), GalNAcαProNHCbz (5 mM), MgCl₂ (20 mM), LpCLS (0.5 mg/mL), and MBP-AbGtr18-His₆ (0.5 mg/mL). HRMS and UHPLC were used for product detection and yield determination.

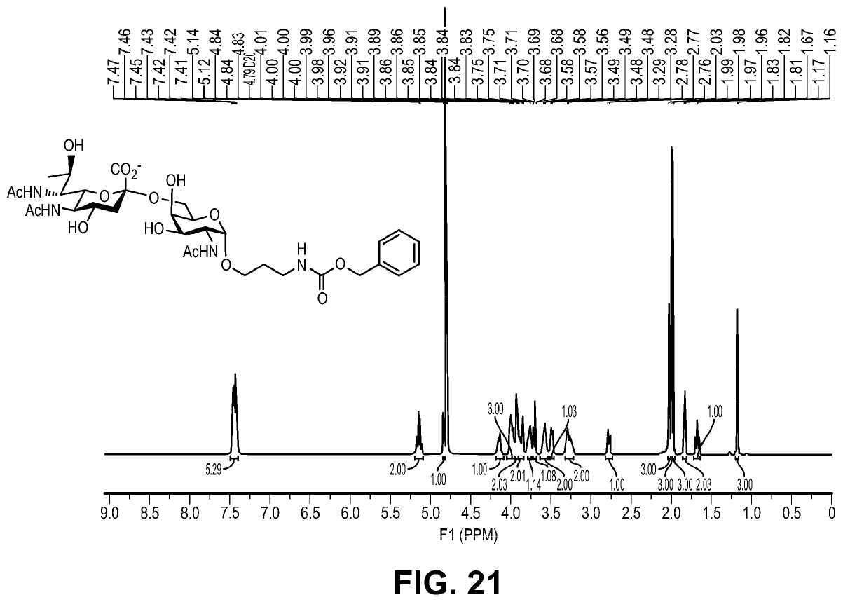

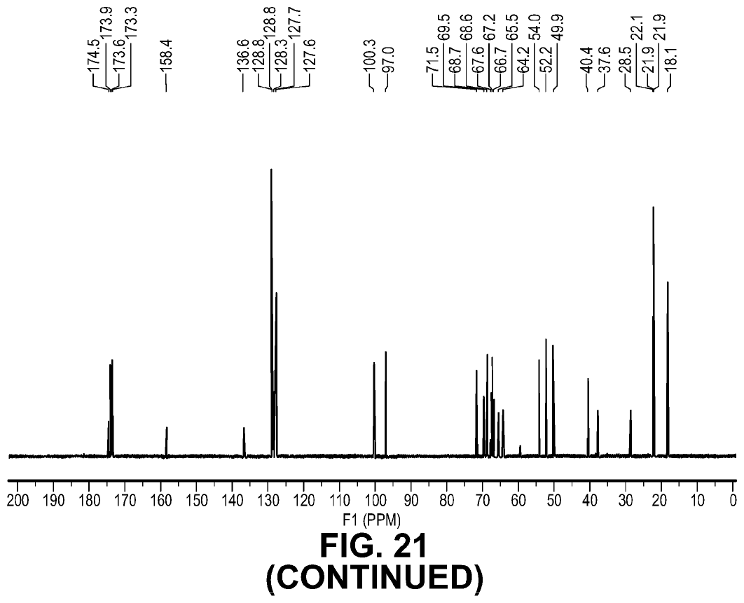

OP3E synthesis of disaccharide Leg5,7Ac₂α2-6GalNAcαProNHCbz. Preparative-scale synthesis was carried out in an aqueous solution (11 mL) containing 6deoxyMan2,4diNAc (31 mg, 0.125 mmol), sodium pyruvate (69 mg, 0.63 mmol), CTP (79 mg, 0.15 mmol), GalNAcαProNHCbz (40 mg, 0.097 mmol), MgCl₂ (20 mM), PmAldolase (32 mg), LpCLS (3 mg), and MBP-AbGtr18-His₆ (3 mg) at 30° C. for 16 hours with agitation at 180 rpm. The product formation was monitored by UHPLC. After the reaction was completed, the reaction was quenched by adding the same volume (11 mL) of ice-cold methanol. The mixture was incubated at 4° C. for 30 minutes and centrifuged to remove precipitates. The supernatant was concentrated, and the residue was purified using a 37 g ODS-SM column (50 mM, 120 A, Yamazen) on a CombiFlash® Rf 200i system to obtain the pure product as a white amorphous powder (66.4 mg, 94%). ¹H NMR (600 MHz, D₂O) δ 7.47-7.41 (m, 5H), 5.13 (q, J=12.7 Hz, 2H), 4.84 (d, J=2.9 Hz, 1H), 4.15 (dd, J=11.1, 3.6 Hz, 1H), 4.02-3.95 (m, 3H), 3.92 (d, J=10.0 Hz, 2H), 3.90-3.82 (m, 2H), 3.78-3.74 (m, 1H), 3.69 (td, J=10.1, 1.4 Hz, 1H), 3.60-3.56 (m, 2H), 3.48 (dt, J=11.0, 5.7 Hz, 1H), 3.32-3.24 (m, 2H), 2.77 (dd, J=12.4, 4.2 Hz, 1H), 2.03 (s, 3H), 1.99 (d, J=1.2 Hz, 3H), 1.97 (d, J=1.2 Hz, 3H), 1.82 (p, J=6.0 Hz, 2H), 1.67 (t, J=12.2 Hz, 1H), 1.17 (d, J=6.1 Hz, 3H); ¹³C NMR (150 MHz, D₂O) δ 174.5, 173.9, 173.6, 173.3, 158.4, 136.6, 128.8, 128.8, 128.3, 127.7, 127.6, 100.3, 97.0, 71.5, 69.5, 68.7, 68.6, 67.6, 67.2, 66.7, 65.5, 64.2, 54.0, 52.2, 49.9, 40.4, 37.6, 28.5, 22.1, 21.9, 21.9, 18.1; HRMS (ESI-Orbitrap) m/z: [M−H]⁻ calculated for C₃₂H₄₈N₄O₁₅ 727.3043; found 727.3042. The ¹H and ¹³C NMR spectra are shown in FIG. 21.

One-pot three-enzyme (OP3E) synthesis of disaccharide Leg5Ac7N₃α2-6GalNAcαProNHCbz. Similarly, Leg5Ac7N₃α2-6GalNAcαProNHCbz was synthesized from GalNAcαProNHCbz and 6deoxyManNAc4N₃ using OP3E containing PmAldolase, LpCLS, and MBP-AbGtr18-His₆ with a 99% yield (Scheme 5).

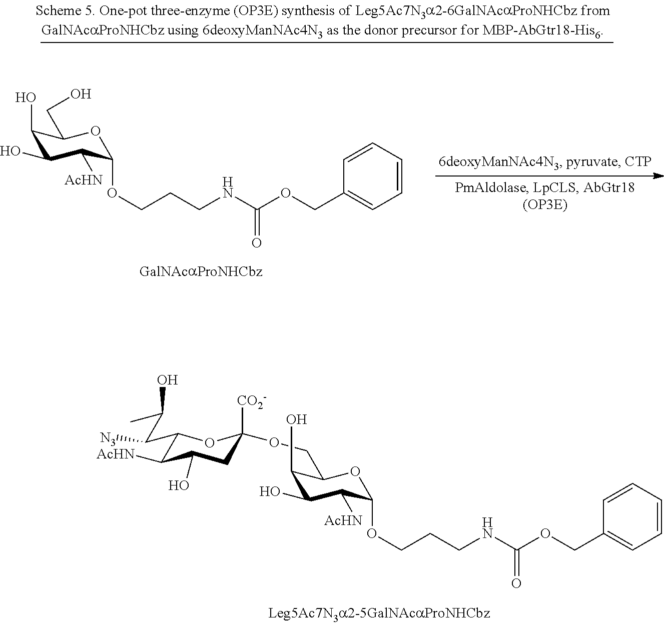

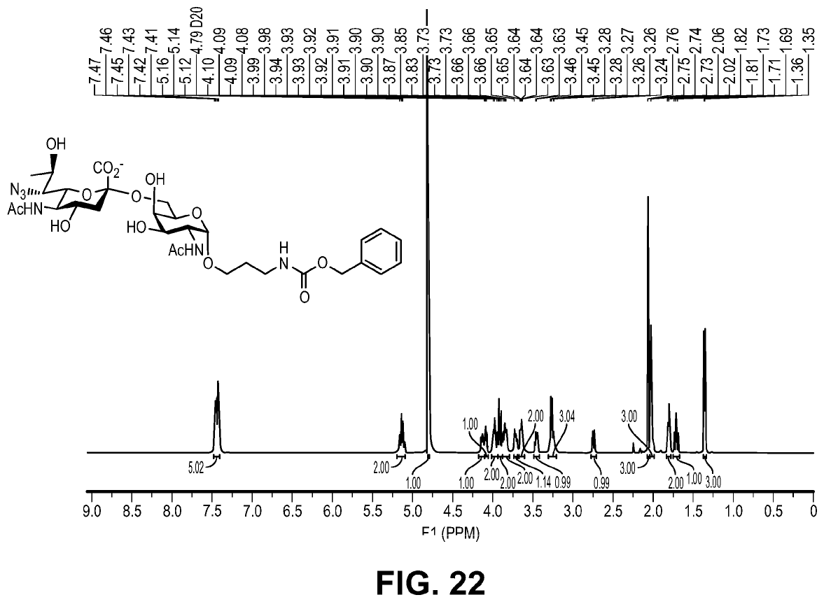

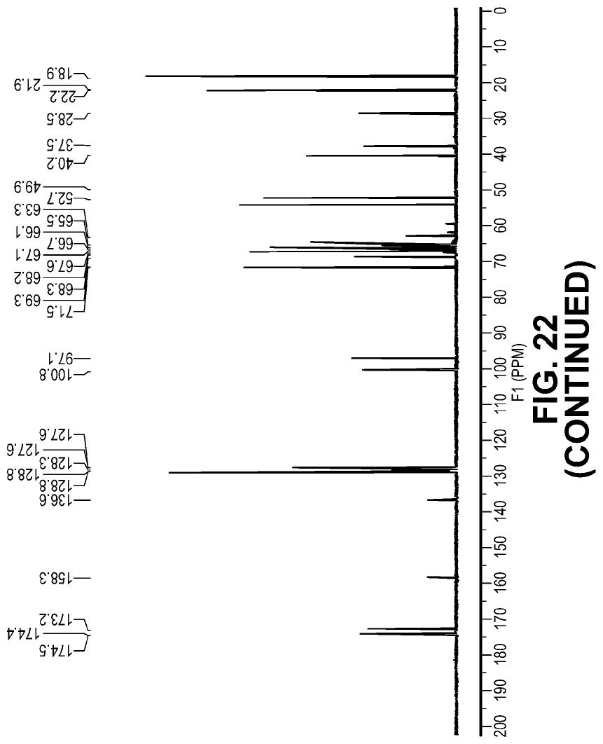

Preparative-scale synthesis was carried out at 30° C. in an aqueous solution (10 mL) containing 6deoxyManNAc4N₃ (35 mg, 0.15 mmol), sodium pyruvate (83.6 mg, 0.75 mmol), and PmAldolase (13 mg) for 12 hours to pre-generate Leg5Ac7N₃. Then PmAldolase (16 mg), CTP (95 mg, 0.18 mmol). MgCl₂ (20 mM), and LpCLS (6 mg) were added, and the reaction mixture was incubated at 30° C. for another 2 hours. GalNAcαProNHCbz (50 mg, 0.12 mmol) and MBP-AbGtr18-His₆ (7 mg) were then added and the resulting reaction mixture (18 mL) was incubated at 30° C. for 7.5 hours with agitation at 180 rpm. The product formation was monitored by UHPLC. After the reaction was completed, the reaction was quenched by adding the same volume (18 mL) of ice-cold methanol. The mixture was incubated at 4° C. for 30 minutes and centrifuged to remove precipitates. The supernatant was concentrated, and the residue was purified using a 37 g ODS-SM column (50 mM, 120 A, Yamazen) on a CombiFlash® Rf 200i system to obtain the pure product as a white amorphous powder (85.6 mg, 99%). ¹H NMR (600 MHz, D₂O) δ 7.47-7.44 (m, 5H), 5.13 (q, J=12.8 Hz, 2H), 4.79 (d, J=2.9 Hz, 1H) [masked by D₂O peak at 4.79], 4.14 (dd, J=11.1, 3.7 Hz, 1H), 4.09 (tt, J=7.0, 3.5 Hz, 1H), 3.98-3.95 (m, 2H), 3.95-3.89 (m, 2H), 3.88-3.80 (m, 2H), 3.74-3.68 (m, 1H), 3.67-3.60 (m, 2H), 3.45 (dt, J=10.7, 5.9 Hz, 1H). 3.32-3.20 (m, 3H), 2.74 (dd, J=12.5, 4.4 Hz, 1H), 2.06 (s, 3H), 2.02 (d, J=1.2 Hz, 3H), 1.81 (p, J=6.5 Hz, 2H), 1.71 (t, J=12.2 Hz, 1H), 1.36 (d, J=6.2 Hz. 3H); ¹³C NMR (150 MHz, D₂O) δ 174.5, 174.4, 173.2, 158.3, 136.6, 128.8, 128.8, 128.3, 127.6, 127.6, 100.8, 97.1, 71.7, 69.3, 68.3, 68.2, 67.6, 67.1, 66.7, 66.1, 65.5, 63.3, 52.7, 49.9, 40.2, 37.5, 28.5, 22.2, 21.9, 18.9. HRMS (ESI-Orbitrap) m/z: [M−H]⁻ calculated for C₃₀H₄₄N₆O₁₄ 711.2843; found 711.2841. The ¹H and ¹³C NMR spectra are shown in FIG. 22.

One-pot three-enzyme (OP3E) syntheses of disaccharide Leg5,7diN₃α2-6GalNAcαProNHCbz. Synthesis of Leg5,7diN₃α2-6GalNAcαProNHCbz from GalNAcαProNHCbz was achieved with a 36.4% yield using an OP3E containing PmAldolase, NmCSS and MBP-AbGtr18-His₆ (Scheme 6). Each reaction was carried out at 30° C. for 20 hours in an aqueous solution (10 L) containing 6deoxyMan2,4diN₃ (7 mM), sodium pyruvate (35 mM), CTP (10 mM), GalNAcαProNHCbz (5 mM), MgCl₂ (20 mM), PmAldolase (0.8 mg/mL), NmCSS (0.4 mg/mL), and MBP-AbGtr18-His₆ (0.35 mg/mL). HRMS and UHPLC were used for product detection and yield determination.

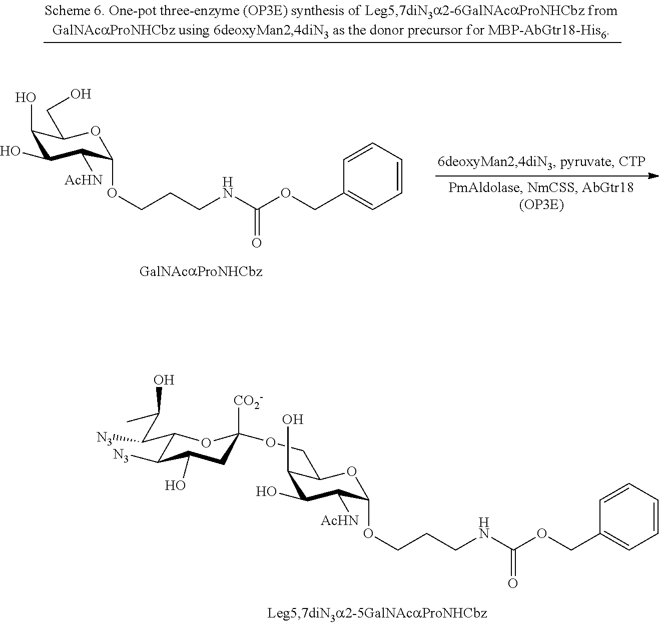

One-pot three-enzyme (OP3E) syntheses of disaccharide Leg5N₃7Acα2-6GalNAcαProNHCbz. Synthesis of Leg5N₃7Acα2-6GalNAcαProNHCbz from GalNAcαProNHCbz was achieved using the OP3E containing PmAldolase, LpCLS and MBP-AbGtr18-His₆ (Scheme 7) with an estimated 20% yield. Each reaction was carried out at 30° C. for 20 hours in an aqueous solution (10 L) containing 6deoxyMan2N₃4NAc (4 mM), sodium pyruvate (20 mM), CTP (4.5 mM), GalNAcαProNHCbz (2 mM), MgCl₂ (20 mM), PmAldolase (0.8 mg/ml), LpCLS (0.2 mg/mL), and MBP-AbGtr18-His₆ (0.2 mg/mL). HRMS was used for product detection and yield estimation.

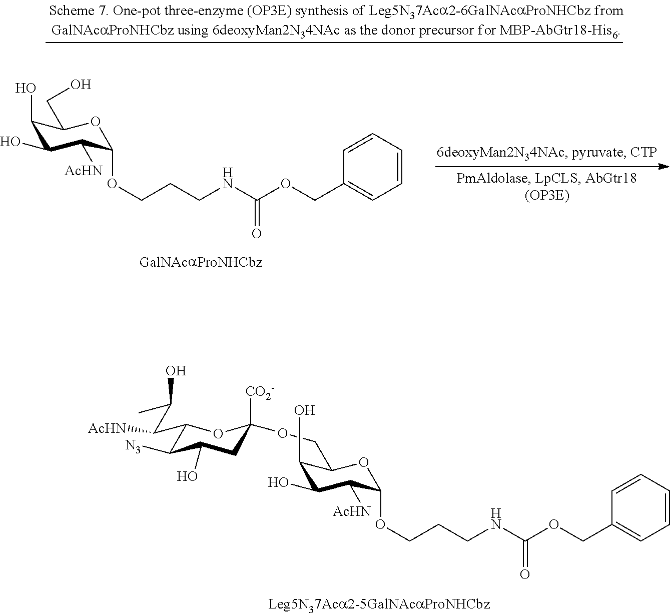

Synthesis of 6deoxyMan2,4diNAc (5) and 6deoxyManNAc4N₃ (8) as precursors for Leg5,7Ac₂ and Leg5Ac7N₃, respectively. 6deoxyMan2,4diNAc (5) was synthesized and the synthesis of 6deoxyManNAc4N₃ (8) was achieved as shown in Scheme 8.

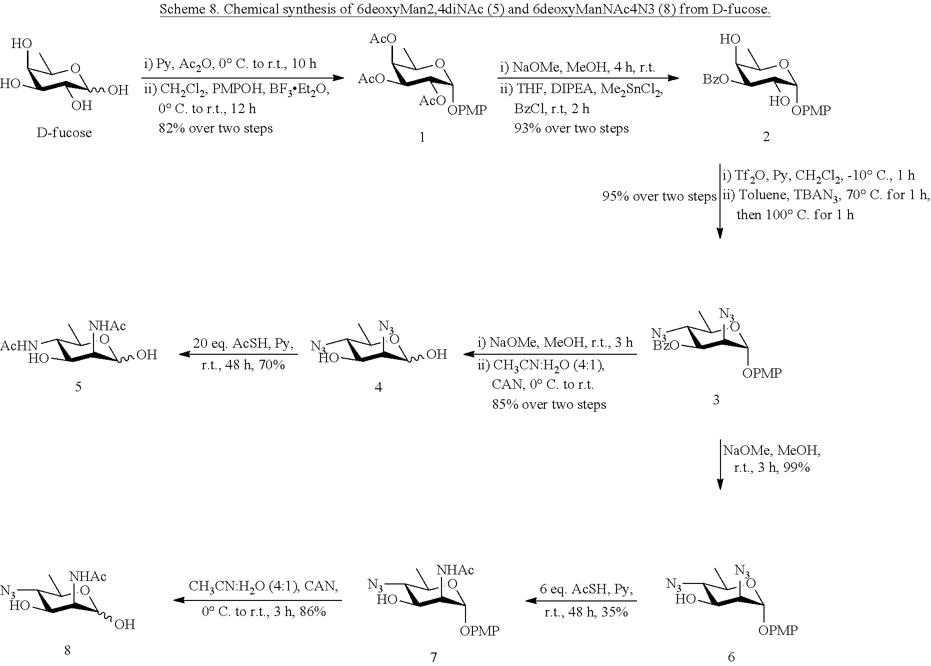

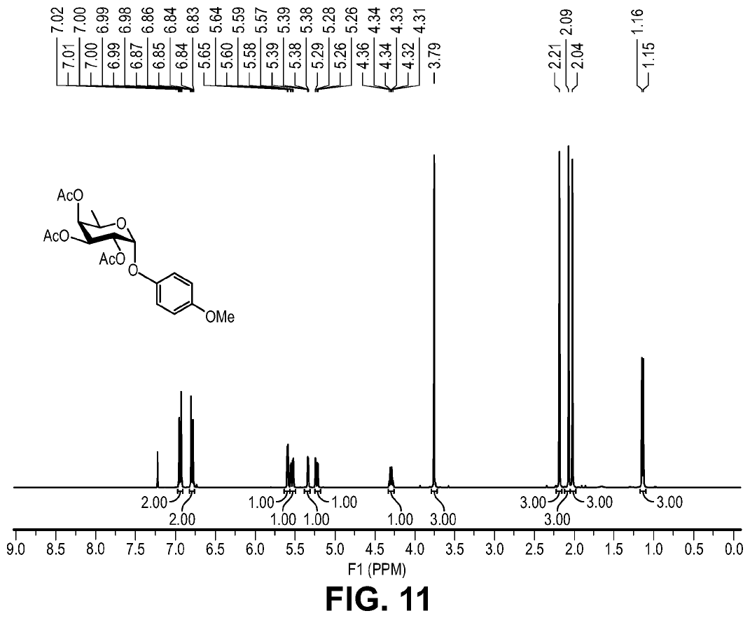

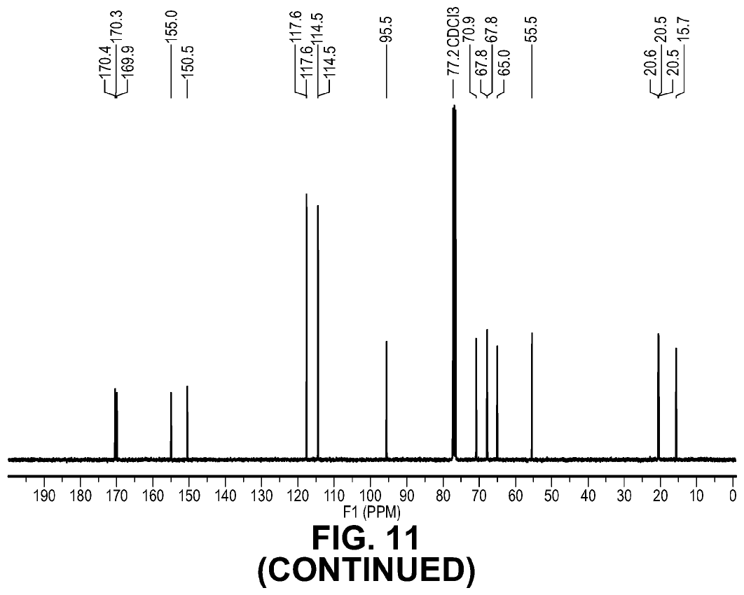

p-Methoxyphenyl-2,3,4-tri-O-acetyl-α-D-fucopyranoside (1). To a solution of D-fucose (7 g, 42.64 mmol) in 50 mL pyridine at 0° C. was added 35 mL acetic anhydride dropwise. After stirring at 0° C. for 1 hour, the mixture was allowed to warm to room temperature and stirred for a total of 12 hours. The solvent was removed in vacuo and co-evaporated with 50 mL of toluene 3 times. The peracetylated D-fucose was dried in vacuo for 6 hours and directly used for next step without further purification. Peracetate (14 g, 42.13 mmol) and p-methoxyphenol (7.84 g, 63.19 mmol) were added in anhydrous CH₂Cl₂ (100 mL) containing 4 Å molecular sieves (5 g) under nitrogen atmosphere and stirred for 30 minutes at room temperature. The reaction mixture was then cooled down to 0° C. using an ice bath, stirred for another 10 minutes and BF₃·OEt₂ (10.40 mL, 84.26 mmol) was added dropwise over a period of 15 minutes. After stirring at 0° C. for 2 hours, the mixture was allowed to warm to room temperature and stirred for another 10 hours. After completion of reaction as indicated by thin layer chromatography (TLC), the reaction was quenched by adding Et₃N and filtered over Celite. After filtration, the solvent was removed under reduced pressure and the product was purified by silica gel chromatography using hexane:EtOAc=8:1 (by volume) as an eluent to produce compound 1 (13.86 g, yield 82% in 2 steps) as a white solid. ¹H NMR (400 MHz, CDCl₃) δ 7.03-6.97 (m, 2H), 6.88-6.82 (m, 2H), 5.64 (d, J=3.7 Hz, 1H), 5.59 (dd, J=10.9, 3.4 Hz, 1H), 5.38 (dd, J=3.5, 1.3 Hz, 1H), 5.27 (dd, J=10.9, 3.7 Hz, 1H), 4.33 (dt, J=6.5, 1.3 Hz, 1H), 3.79 (s, 3H), 2.21 (s, 3H), 2.09 (s, 3H), 2.04 (s, 3H), 1.16 (d, J=6.5 Hz, 3H); ¹³C NMR (100 MHz, CDCl₃) δ 170.4, 170.3, 169.9, 155.0, 150.5, 117.6, 117.6, 114.5, 114.5, 95.5, 70.9, 67.8, 67.8, 65.0, 55.5, 20.6, 20.5, 20.5, 15.7; HRMS (ESI-Orbitrap) m/z: [M+Na]⁺ calculated for C₁₉H₂₄O₉Na 419.1318; found 419.1312. The ¹H and ¹³C NMR spectra are shown in FIG. 11.

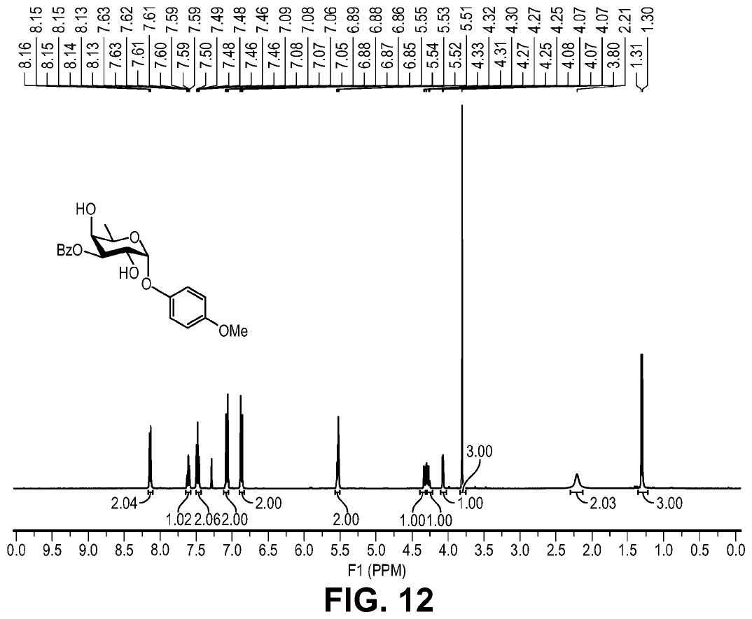

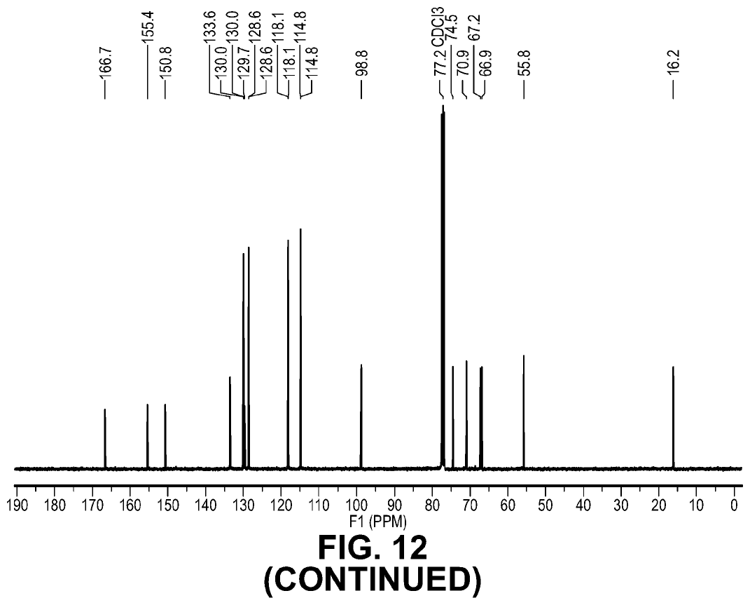

p-Methoxyphenyl-3-O-benzoyl-α-D-fucopyranoside (2). To a solution of 4-methoxyphenyl-2,3,4-tri-O-acetyl-α-D-fucopyranoside 1 (13 g, 32.80 mmol) in methanol:CH₂Cl₂ (1:1) (60 mL) was added 30% sodium methoxide in methanol (1 mL) at room temperature. After 4 hours, the reaction mixture was neutralized with Dowex 50W (H⁺), filtered and concentrated under reduced pressure. This intermediate was dried in vacuo for 6 hours and used in the next step without further purification. To a stirred solution of intermediate (8.86 g, 32.78 mmol) in anhydrous THF (50 mL), Me₂SnCl₂ (360 mg, 1.64 mmol) and DIPEA (N,N-diisopropylethylamine) (22.9 mL, 131.12 mmol) were added and stirred for 15 minutes. BzCl (4.19 mL, 36.06 mmol) was then added dropwise and after 1 hour the reaction mixture was quenched with 1M HCl (70 mL) and extracted with EtOAc, dried over Na₂SO₄. After filtration, the solvent was removed under reduced pressure and the product was purified by silica gel chromatography using hexane:EtOAc=4:1 (by volume) as an eluent to produce compound 2 (11.41 g, yield 93% in 2 steps) as a white solid. ¹H NMR (400 MHz, CDCl₃) δ 8.18-8.10 (m, 2H), 7.65-7.57 (m, 1H), 7.51-7.44 (m, 2H), 7.11-7.03 (m, 2H), 6.92-6.84 (m, 2H), 5.54 (dd, J=3.5, 3.1 Hz, 1H), 5.52 (t, J=3.5 Hz, 1H), 4.32 (dd, J=10.3, 3.8 Hz, 1H), 4.26 (dd, J=6.4, 1.2 Hz, 1H), 4.07 (dd, J=3.2, 1.2 Hz, 1H), 3.80 (s, 3H), 2.21 (s, 2H), 1.31 (d, J=6.6 Hz, 3H); ¹³C NMR (100 MHz, CDCl₃) δ 166.7, 155.4, 150.8, 133.6, 130.0, 130.0, 129.7, 128.6, 128.6, 118.1, 118.1, 114.8, 114.8, 98.8, 74.5, 70.9, 67.2, 66.9, 55.8, 16.2; HRMS (ESI-Orbitrap) m/z: [M+Na]⁺ calculated for C₂₀H₂₂O₇Na 397.1263; found 397.1258. The ¹H and ¹³C NMR spectra are shown in FIG. 12.

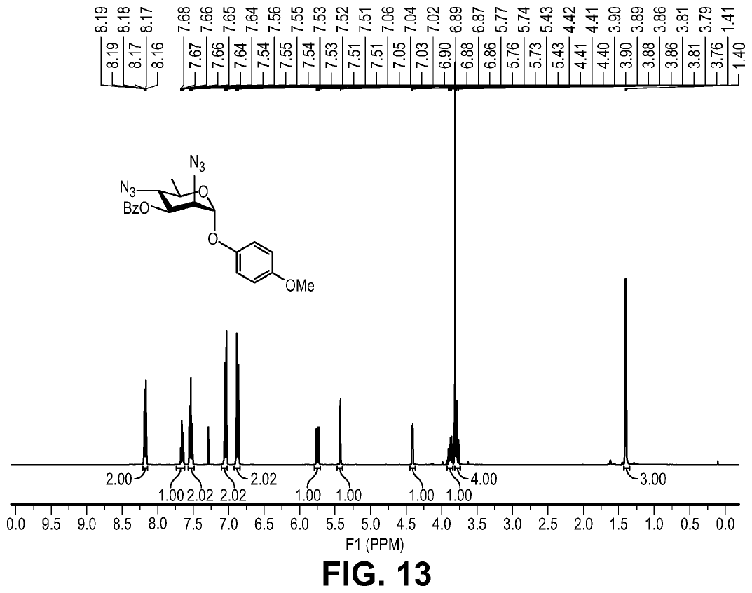

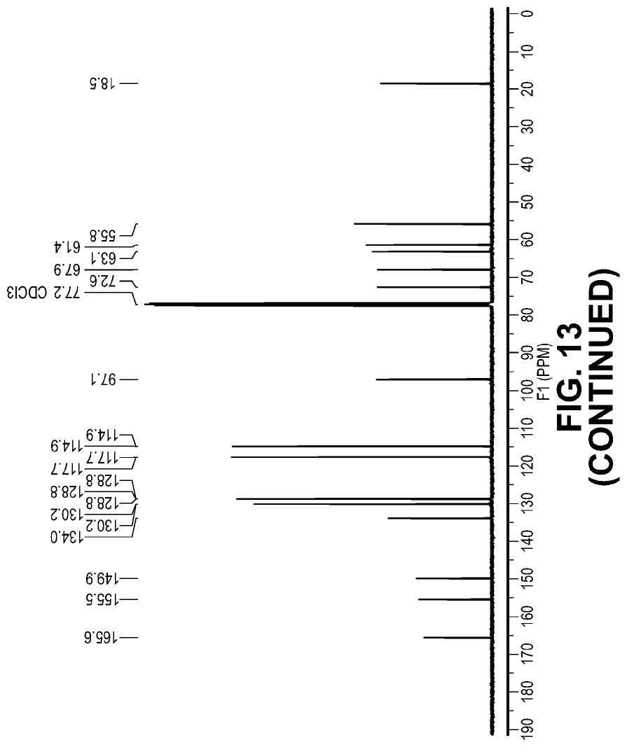

p-Methoxyphenyl-2,4-di-azido-3-O-benzoyl-2,4,6-trideoxy-α-D-mannopyranoside (3). To a solution of compound 2 (5 g, 13.36 mmol) in anhydrous CH₂Cl₂ (50 mL) and anhydrous pyridine (10 mL) at −10° C. trifluoromethanesulfonic anhydride (13.5 mL, 80.13 mmol) was added. Temperature was slowly increased to 0° C. over a period of 1 hour. The reaction was diluted with the addition of 70 mL of CH₂Cl₂. The organic layer was washed with 1 μM HCl, saturated NaHCO₃, and brine solution, and then dried over anhydrous Na₂SO₄. After filtration, the solvent was removed under reduced pressure at room temperature, was dried in vacuo for 2 hours and directly used for next step without further purification. To a solution of the 2,4-bistriflate in anhydrous toluene (70 mL) at 70° C., tetrabutylammonium azide (11.40 g, 40.08 mmol) was added and the mixture was stirred for 1 hour. The temperature was then increased to 100° C. and the mixture was stirred for another 1 hour. Then the solvent was removed and the condensed mixture was diluted with 75 mL of CH₂C₁₂. The organic layer was washed with brine solution and dried over anhydrous Na₂SO₄. After filtration, the solvent was removed under reduced pressure and the product was purified by silica gel chromatography using toluene:hexane=25:1 (by volume) as an eluent to produce compound 3 (5.39 g, yield 95% over 2 steps) as a white solid. ¹H NMR (400 MHz, CDCl₃) δ 8.24-8.14 (m, 2H), 7.69-7.60 (m, 1H), 7.59-7.47 (m, 2H), 7.10-6.97 (m, 2H), 6.91-6.82 (m, 2H), 5.75 (dd, J=10.1, 3.7 Hz, 1H), 5.43 (d, J=1.8 Hz, 1H), 4.41 (dd, J=3.7, 1.8 Hz, 1H), 3.92-3.84 (m, 1H), 3.81 (s, 3H), 3.77 (t, J=10.1 Hz, 1H), 1.41 (d, J=6.1 Hz, 3H); ¹³C NMR (100 MHz, CDCl₃) δ 165.6, 155.5, 149.9, 134.0, 130.2, 130.2, 128.8, 128.8, 128.8, 117.7, 117.7, 114.9, 114.9, 97.1, 72.6, 67.9, 63.1, 61.4, 55.8, 18.5; HRMS (ESI-Orbitrap) m/z: [M+Na]⁺ calculated for C₂₀H₂₀N₆O₅Na 447.1393; found 447.1405. The ¹H and ¹³C NMR spectra are shown in FIG. 13.

2,4-Diazido-2,4,6-trideoxy-D-mannose (α:β=1.0:0.8) (4). To a solution of compound 3 (3 g, 7.07 mmol) in anhydrous methanol (30 mL) was added 30% sodium methoxide in methanol (300 μL) at room temperature. After 3 hours, the reaction mixture was neutralized with Dowex 50W (H¹), filtered and concentrated under reduced pressure. This intermediate was dried in vacuo and used in the next step without further purification.

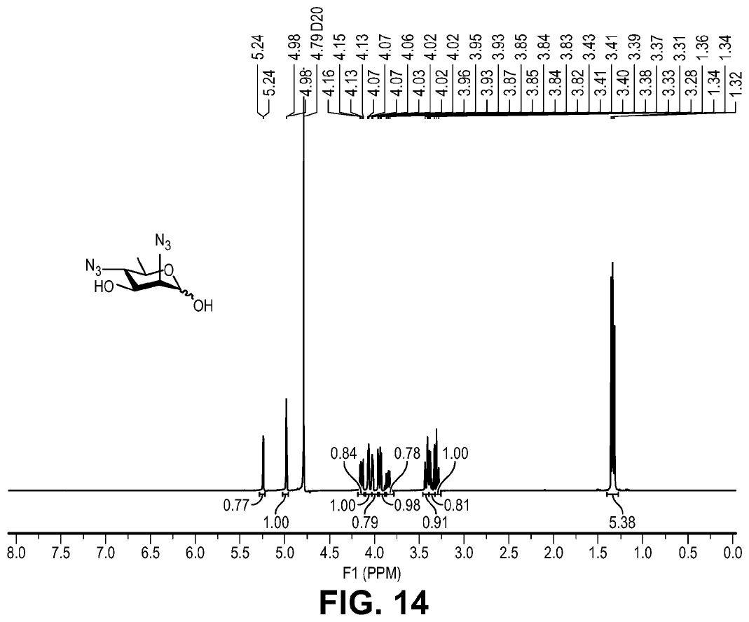

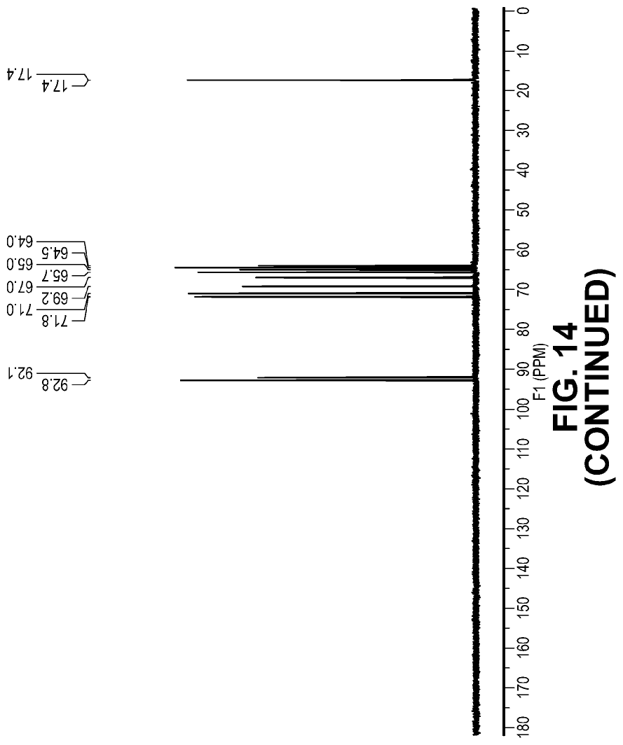

To a solution of the 2,4-diazido intermediate in 40 mL of acetonitrile:water=4:1 (by volume) at 0° C., ceric ammonium nitrate (11.60 g, 21.17 mmol) was added and the reaction mixture was stirred for 1 hour. The reaction was warmed up to room temperature and was stirred for another 3 hours. The acetonitrile was then removed under reduced pressure at room temperature and diluted with 100 mL of ethyl acetate. The organic layer was washed with water saturated NaHCO₃, and brine solution, and then dried over anhydrous Na₂SO₄. After filtration, the solvent was removed under reduced pressure and the product was purified by silica gel chromatography using a mixed solvent (Hexane:EtOAc=2:1, by volume) as an eluent to produce compound 4 (1.28 g, yield 85% over 2 steps) as a reddish solid. ¹H NMR (400 MHz, D₂O) δ 5.24 (d, J=1.7 Hz, 1H), 4.98 (d, J=1.4 Hz, 1H), 4.14 (dd, J=10.0, 3.8 Hz, 1H), 4.07 (dd, J=3.7, 1.3 Hz, 1H), 4.02 (dd, J=3.8, 1.7 Hz, 1H), 3.94 (dd, J=9.7, 3.7 Hz, 1H), 3.89-3.79 (m, 1H), 3.44-3.39 (m, 1H), 3.39-3.37 (m, 1H), 3.32 (q, J=10.2, 9.8 Hz, 1H), 1.35 (d, J=6.1 Hz, 3H), 1.33 (d, J=6.3 Hz, 3H); ¹³C NMR (100 MHz, D₂O) δ 92.8, 92.1, 71.8, 71.0, 69.2, 67.0, 65.7, 65.0, 64.5, 64.0, 17.4, 17.4; HRMS (ESI-Orbitrap) m/z: [M+Cl]⁻ calculated for C₆H₁₀N₆O₃Cl 249.0508; found 249.0509. The ¹H and ¹³C NMR spectra are shown in FIG. 14.

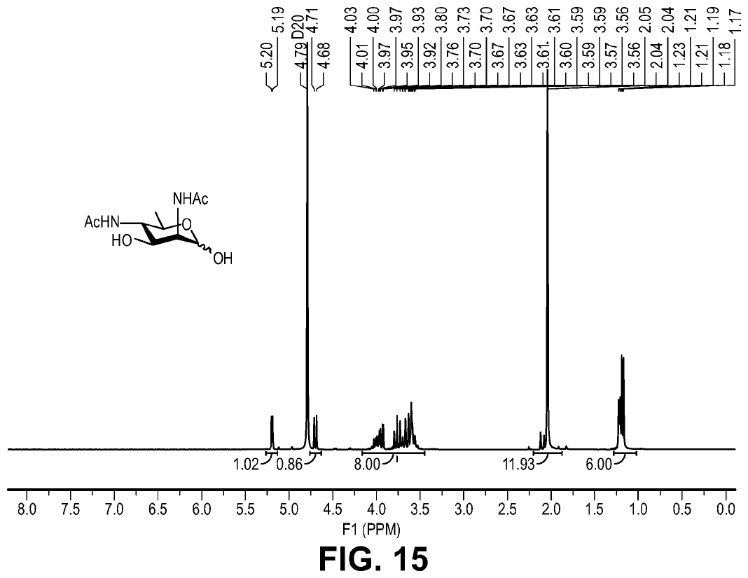

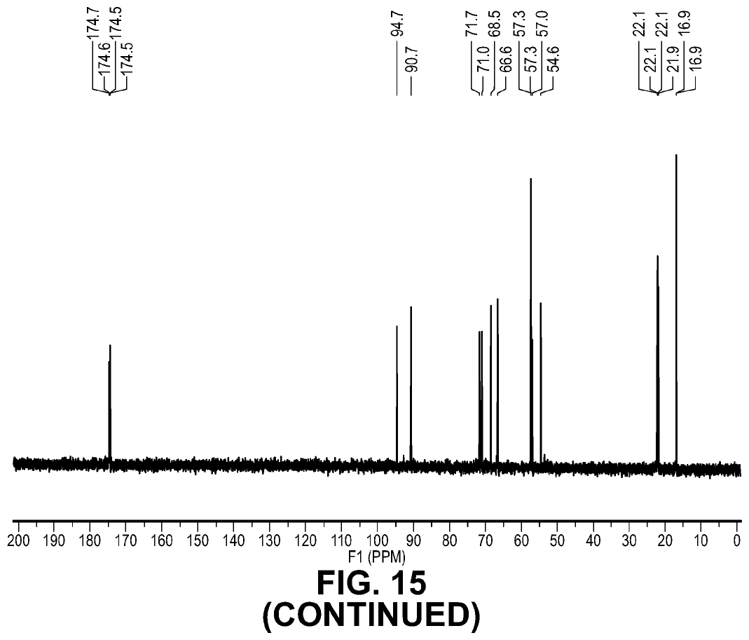

6deoxyManNAc4NAc (α:β=1.0:1.1) (5). To a solution of compound 4 (1 g, 4.67 mmol) in pyridine (20 mL) was added thioacetic acid (3.3 mL, 46.69 mmol) under argon at room temperature, and the mixture was stirred for 48 hours. The resulting product was purified by silica gel chromatography using a mixed solvent (EtOAc:MeOH=10:1, by volume) as an eluent to produce compound 5 (805 mg, yield 73%) as a white solid. ¹H NMR (400 MHz, D₂O) δ 5.19 (d, J=3.5 Hz, 1H), 4.70 (d, J=8.3 Hz, 1H), 4.03-3.56 (m, 8H), 2.05-2.04 (m, 12H), 1.23-1.17 (m, 6H); ¹³C NMR (100 MHz, D₂O) δ 174.7, 174.6, 174.5, 174.5, 94.7, 90.7, 71.7, 71.0, 68.5, 66.6, 57.3, 57.3, 57.0, 54.6, 22.1, 22.1, 22.1, 21.9, 16.9, 16.9; HRMS (ESI-Orbitrap) m/z: [M+Cl]⁻ calculated for C₁₀H₁₈N₂O₅Cl 281.0910; found 281.0913. The ¹H and ¹³C NMR spectra are shown in FIG. 15.

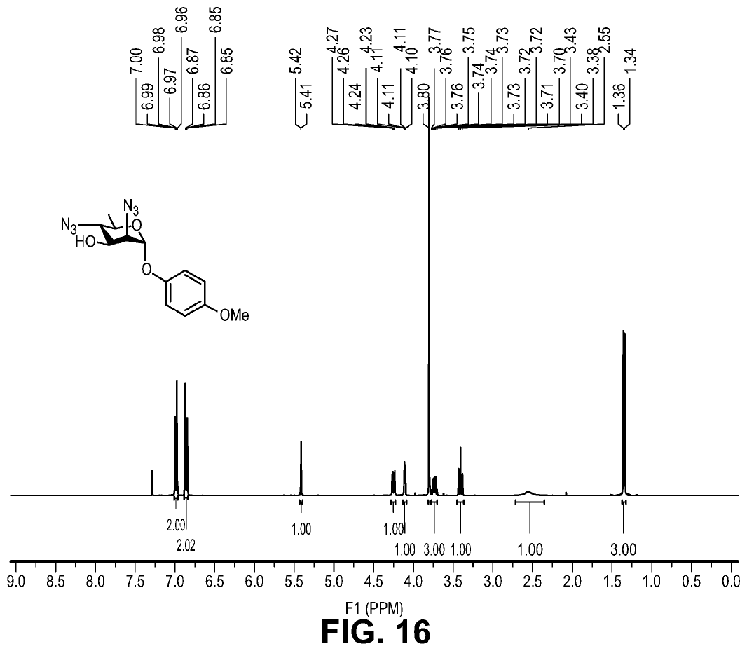

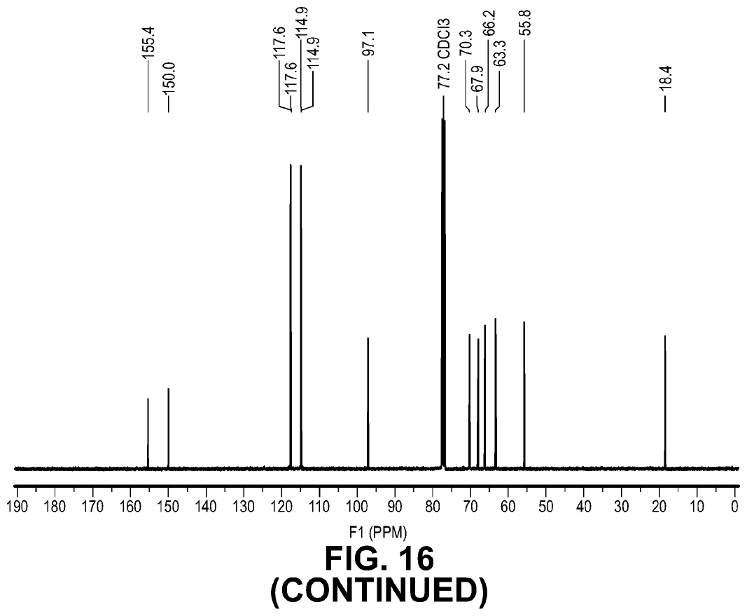

p-Methoxyphenyl-2,4-di-azido-2,4,6-trideoxy-α-D-mannopyranoside (6). To a solution of compound 3 (2 g, 4.71 mmol) in anhydrous methanol (20 mL) was added 30% sodium methoxide in methanol (200 L) at room temperature. After 3 hours, the reaction mixture was neutralized with Dowex 50W (H⁺), filtered and concentrated under reduced pressure. The crude product was purified by silica gel chromatography using a mixed solvent (Hexane:EtOAc=7:3, by volume) as an eluent to produce compound 6 (1.49 g, yield 99%) as a white solid. ¹H NMR (400 MHz, CDCl₃) δ 7.03-6.93 (m, 2H), 6.90-6.81 (m, 2H), 5.41 (d, J=1.7 Hz, 1H), 4.25 (dd, J=9.8, 3.9 Hz, 1H), 4.11 (dd, J=3.9, 1.7 Hz, 1H), 3.80 (s, 3H), 3.78-3.69 (m, 1H), 3.40 (t, J=9.9 Hz, 1H), 2.55 (s, 1H), 1.35 (d, J=6.2 Hz, 3H); ¹³C NMR (100 MHz, CDCl₃) δ 155.4, 150.0, 117.6, 117.6, 114.9, 114.9, 97.1, 70.3, 67.9, 66.2, 63.3, 55.8, 18.4; HRMS (ESI-Orbitrap) m/z: [M+Cl]⁻ calculated for C₁₃H₁₆N₆O₄Cl 355.0927; found 355.0931. The ¹H and ¹³C NMR spectra are shown in FIG. 16.

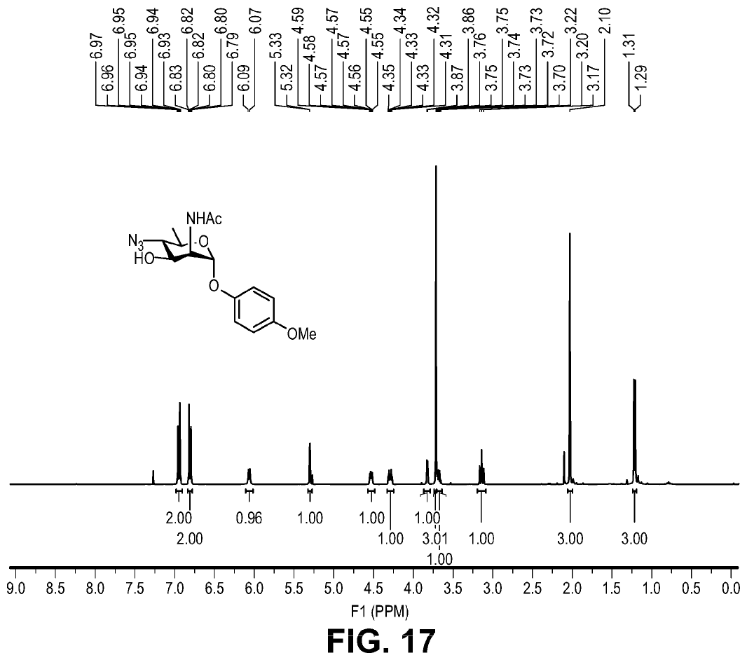

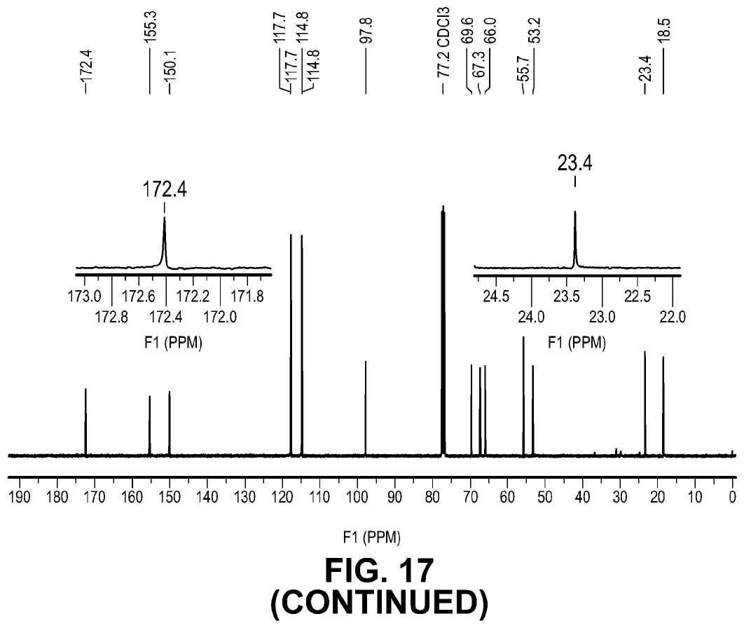

p-Methoxyphenyl-2-acetamido-4-azido-2,4,6-trideoxy-α-D-mannopyranoside (7). To a solution of compound 6 (1 g, 3.12 mmol) in pyridine (15 mL) was added thioacetic acid (440 μL, 6.24 mmol) under argon at room temperature and stirred for 24 hours. After 24 hours, additional thioacetic acid (440 μL, 6.24 mmol) was added and stirred for 12 hours. Then additional thioacetic acid (220 μL, 3.12 mmol) was added and stirred for another 12 hours. The product was purified by silica gel chromatography using a mixed solvent (Hexane:EtOAc:=3:7, by volume) as an eluent to produce compound 7 (367 mg, yield 35%) as a white solid. ¹H NMR (400 MHz, CDCl₃) δ 6.99-6.92 (m, 2H), 6.84-6.77 (m, 2H), 6.08 (d, J=7.9 Hz, 1H). 5.32 (d, J=1.6 Hz, 1H), 4.57 (ddd, J=8.0, 4.5, 1.7 Hz, 1H), 4.33 (dt, J=10.0, 4.1 Hz, 1H), 3.87 (d, J=4.1 Hz, 1H), 3.76 (s, 3H), 3.75-3.69 (m, 1H), 3.20 (t, J=10.1 Hz, 1H), 2.10 (s, 3H), 1.30 (d, J=6.2 Hz, 3H); ¹³C NMR (100 MHz, CDCl₃) δ 172.4, 155.3, 150.1, 117.7, 117.7, 114.8, 114.8, 97.8, 69.6, 67.3, 66.0, 55.7, 53.2, 23.4, 18.5; HRMS (ESI-Orbitrap) m/z: [M+Cl]⁻ calculated for C₁₅H₂₀N₄O₅Cl 335.1361; found 335.1361. The ¹H and 13C NMR spectra are shown in FIG. 17.

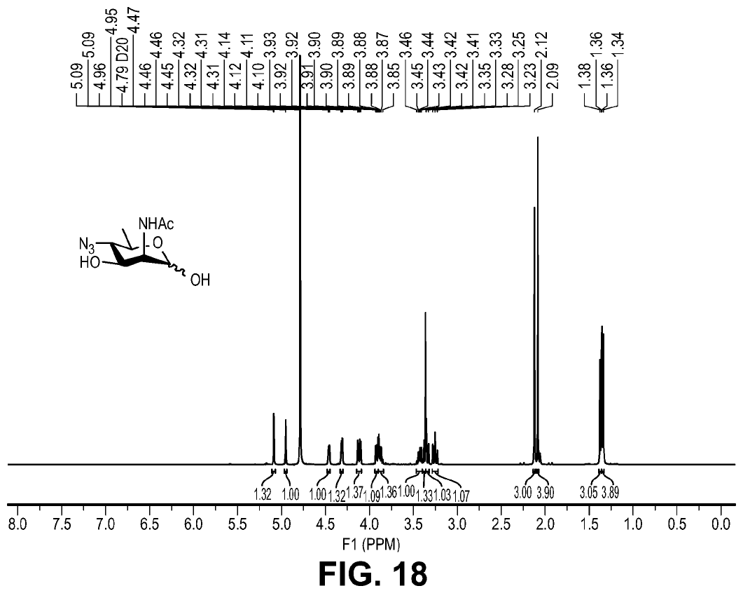

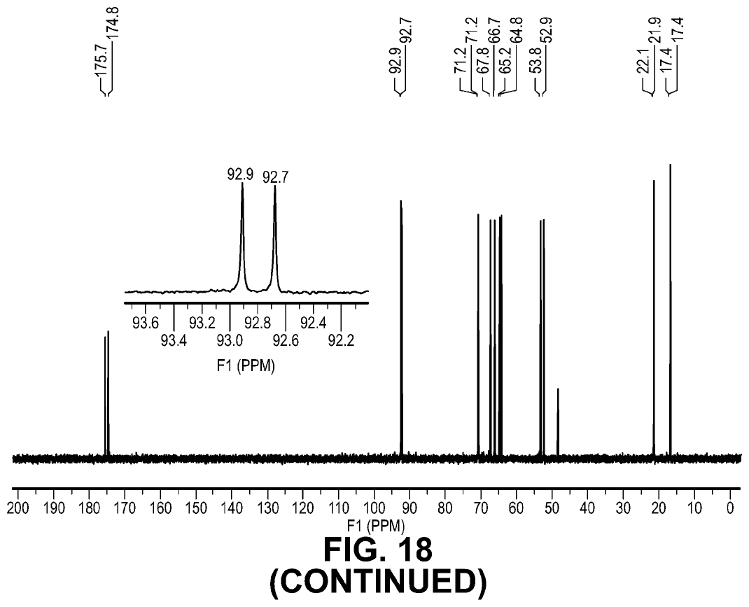

6deoxyManNAc4N₃ (α:β=1.0:1.3) (8). To a solution of compound 7 (700 mg, 2.08 mmol) in 20 mL of acetonitrile:water=4:1 (by volume) at 0° C., ceric ammonium nitrate (3.42 g, 6.24 mmol) was added and the reaction mixture was stirred for 1 hour. The reaction was warmed up to room temperature and was stirred for another 3 hours. The acetonitrile was then removed under reduced pressure at room temperature and diluted with 20 mL of water. The water layer was washed with ethyl acetate (2×20 mL), neutralized with saturated NaHCO₃, and centrifuged. After filtration, the water was removed under reduced pressure and the product was purified by silica gel chromatography using EtOAc:MeOH=9:1 (by volume) as an eluent to produce compound 8 (412 mg, yield 86%) as a reddish solid. ¹H NMR (400 MHz, D₂O) δ 5.09 (d, J=1.6 Hz, 1H), 4.95 (d, J=1.7 Hz, 1H), 4.46 (dd, J=4.4, 1.7 Hz, 1H), 4.32 (dd, J=4.7, 1.6 Hz, 1H), 4.12 (dd, J=10.3, 4.6 Hz, 1H), 3.84-3.94 (m, 2H), 3.44 (dt, J=10.0, 6.2 Hz, 1H), 3.34 (d, J=10.3 Hz, 1H), 3.25 (t, J=10.1 Hz, 1H), 2.12 (s, 3H), 2.09 (s, 3H), 1.37 (d, J=6.2 Hz, 3H), 1.35 (d, J=6.3 Hz, 3H); ¹³C NMR (100 MHz, D₂O) δ 175.7, 174.8, 92.9, 92.7, 71.2, 71.2, 67.8, 66.7, 65.2, 64.8, 53.8, 52.9, 22.1, 21.9, 17.4, 17.4; HRMS (ESI-Orbitrap) m/z: [M+Cl]⁻ calculated for C₈H₁₄N₄O₄Cl 265.0709; found 265.0705. The ¹H and ¹³C NMR spectra are shown in FIG. 18.

Synthesis of 6deoxyMan2N₃4NAc (10) as LegT donor precursor. 6deoxyMan2N₃4NAc (10) was synthesized from compound 6 as shown in Scheme 9.

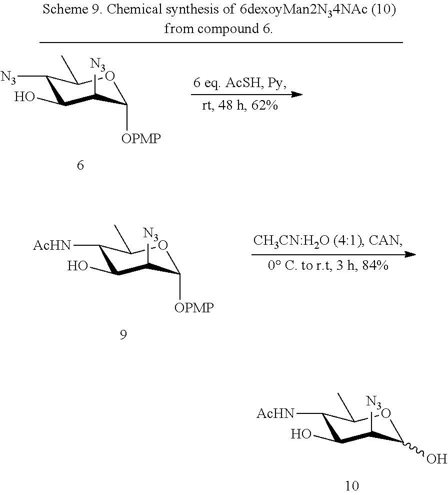

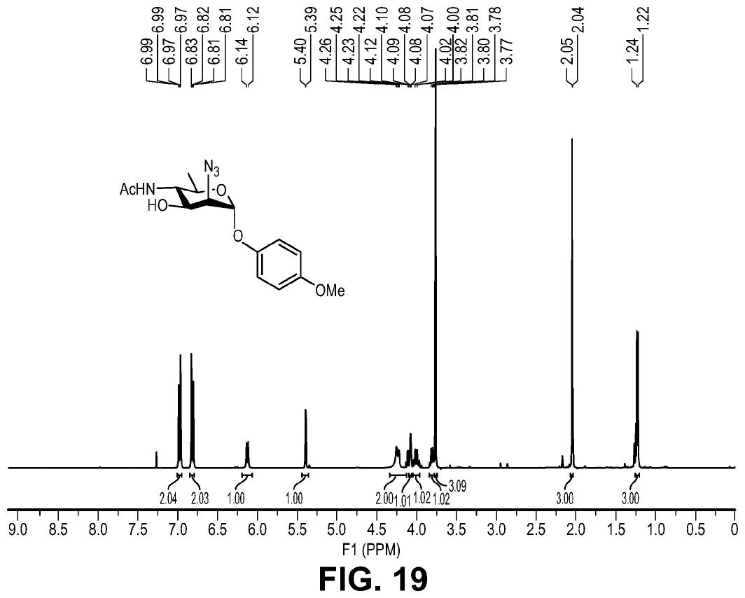

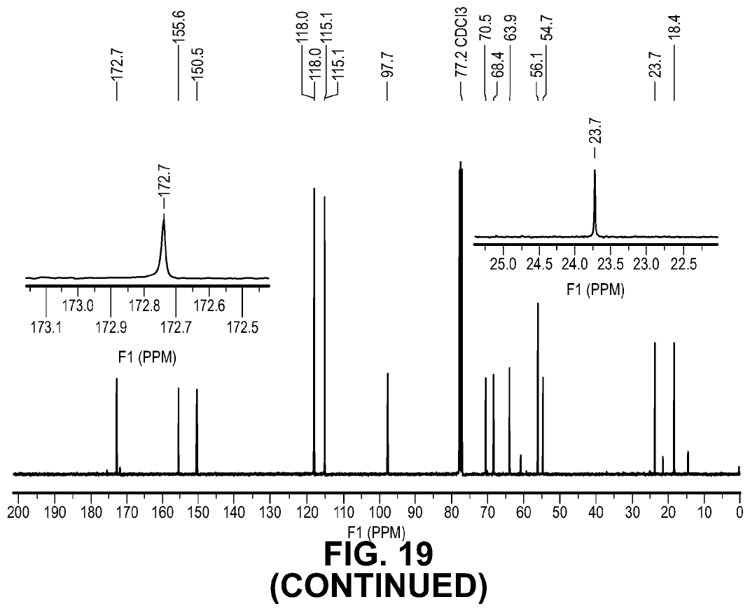

p-Methoxyphenyl-4-acetamido-2-azido-2,4,6-trideoxy-α-D-mannopyranoside (9). To a solution of compound 6 (1 g, 3.12 mmol) in pyridine (15 mL) was added thioacetic acid (440 μL, 6.24 mmol) under argon at room temperature and stirred for 24 h. After 24 h, additional thioacetic acid (440 μL, 6.24 mmol) was added and stirred for 12 h. Then additional thioacetic acid (220 μL, 3.12 mmol) was added and stirred for another 12 h. The product was purified by silica gel chromatography using a mixed solvent (hexane:EtOAc:=1:9, by volume) as an eluent to produce compound 9 (651 mg, yield 62%) as a white solid. ¹H NMR (400 MHz, CDCl₃) δ 7.03-6.93 (m, 2H), 6.85-6.78 (m, 2H), 6.13 (d, J=8.7 Hz, 1H), 5.40 (d, J=1.8 Hz, 1H), 4.25 (s, 1H), 4.24 (dd, J=10.6, 3.8 Hz, 1H), 4.08 (dd, J=3.2, 1.7 Hz, 1H), 4.01 (d, J=9.5 Hz, 1H), 3.80 (dd, J=10.0, 6.2 Hz, 1H), 3.77 (s, 3H), 2.05 (s, 3H), 1.23 (d, J=6.2 Hz, 3H); ¹³C NMR (100 MHz, CDCl₃) δ 172.7, 155.6, 150.5, 118.0, 118.0, 115.1, 115.1, 97.7, 70.5, 68.4, 63.9, 56.1, 54.7, 23.7, 18.4; HRMS (ESI-Orbitrap) m/z: [M+Cl]⁻ Calcd for C₁₅H₂₀N₄O₅Cl 335.1361; found 335.1359. The ¹H and ¹³C NMR spectra are shown in FIG. 19.

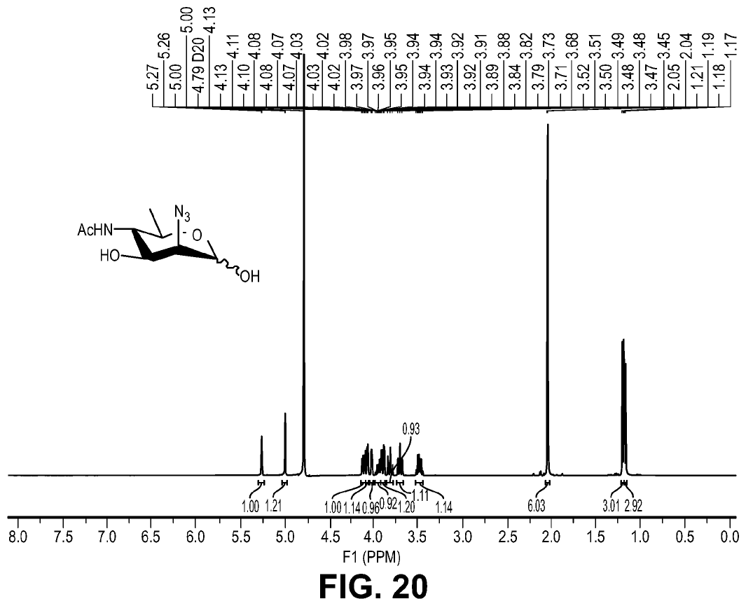

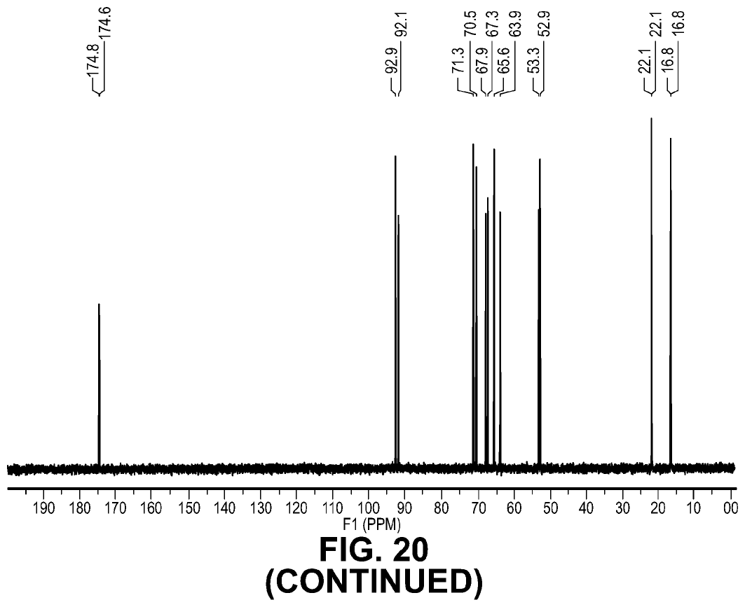

6deoxyMan2N₃4NAc (α:β=1.0:1.2) (10). To a solution of compound 9 (600 mg, 1.78 mmol) in 20 mL of acetonitrile:water=4:1 (by volume) at 0° C., ceric ammonium nitrate (2.93 g, 5.35 mmol) was added and the reaction mixture was stirred for 1 h. The reaction was warmed up to room temperature and was stirred for another 3 h. The acetonitrile was then removed under reduced pressure at room temperature and diluted with 20 mL of water. The water layer was washed with ethyl acetate (2×20 mL), neutralized with saturated NaHCO₃, and centrifuged. After filtration, the water was removed under reduced pressure and the product was purified by silica gel chromatography using EtOAc:MeOH=9:1 (by volume) as an eluent to produce compound 10 (345 mg, yield 84%) as a reddish solid. ¹H NMR (400 MHz, D₂O) δ 5.26 (d, J=1.8 Hz, 1H), 5.00 (d, J=1.4 Hz, 1H), 4.12 (dd, J=10.4, 3.7 Hz, 1H), 4.07 (dd, J=3.7, 1.3 Hz, 1H), 4.03 (dd, J=3.7, 1.8 Hz, 1H), 3.99-3.92 (m, 1H), 3.90 (dd, J=10.5, 3.7 Hz, 1H), 3.82 (t, J=10.3 Hz, 1H), 3.71 (t, J=10.2 Hz, 1H), 3.49 (dq, J=10.0, 6.2 Hz, 1H), 2.05 (s, 3H), 2.04 (s, 3H), 1.20 (d, J=6.2 Hz, 3H), 1.17 (d, J=6.2 Hz, 3H); ¹³C NMR (100 MHz, D₂O) δ 174.8, 174.6, 92.9, 92.1, 71.3, 70.5, 67.9, 67.3, 65.6, 63.9, 53.3, 52.9, 22.1, 22.1, 16.8, 16.8; HRMS (ESI-Orbitrap) m/z: [M+Cl]⁻ Calcd for C₈H₁₄N₄O₄Cl 265.0709; found 265.0706. The ¹H and ¹³C NMR spectra are shown in FIG. 20.

### Example 4. Substrate Specificity Studies

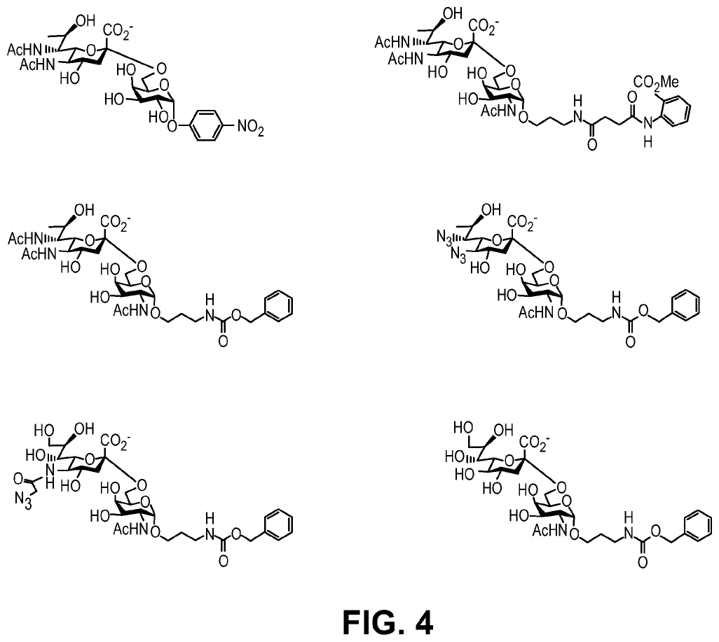

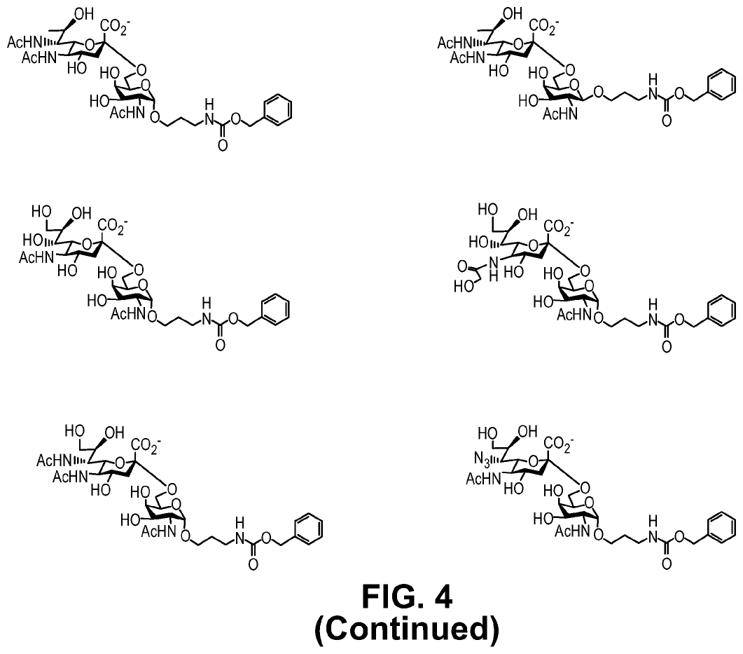

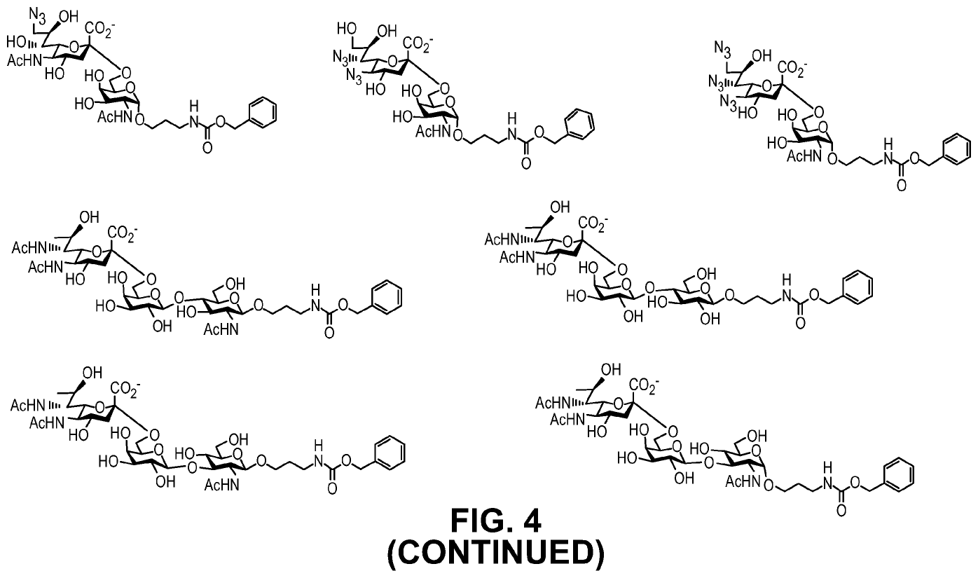

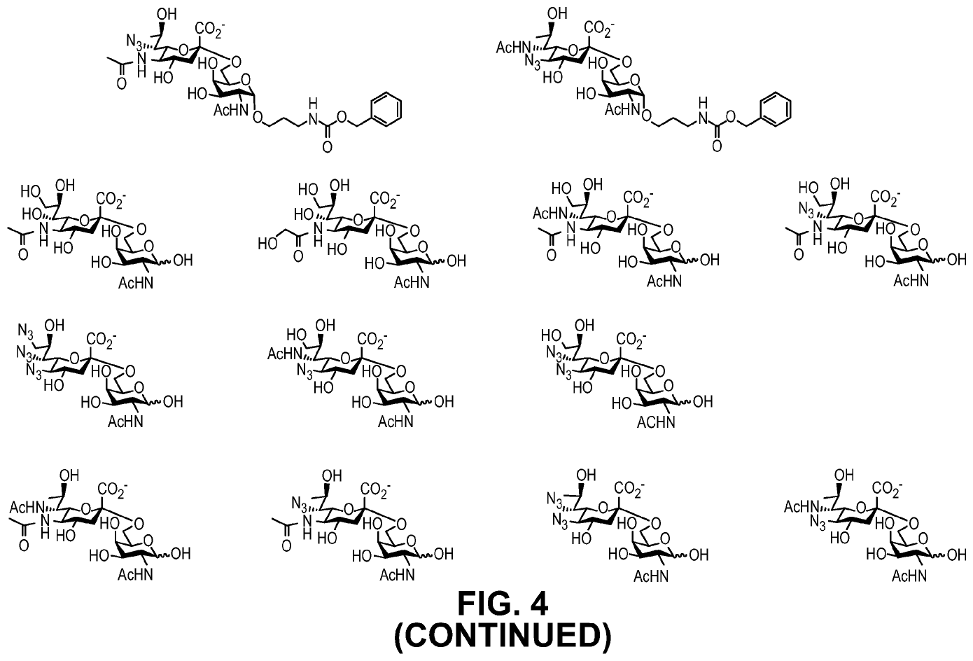

Substrate specificity studies were carried out for AbGtr18 using a high-performance liquid chromatography (HPLC)-based quantitative assay and the structures of products are shown in (FIG. 4). Some monosaccharides and several structurally defined synthetic disaccharides containing a methyl 2-anthranilic acid ester (2AA) tag, a hydrophobic benzyloxycarbonyl (Cbz)-protected propylamine (ProNHCbz) or 4-nitrophenyl tag at the reducing end were used as potential acceptor substrates. As shown in Table 2, monosaccharides GalNAcαOR were well tolerated. The tag could affect the activity of His₆-AbGtr18 to some extent, since it was the most reactive towards GalNAcα2AA while with lower efficiency towards GalNAcαProNHCbz for short time reaction (15 minutes). It was active, but with much lower efficiencies, towards monosaccharides GalNAc3ProNHCbz, GlcNAcαProNHCbz and GalαpNP, and type I glycans Galβ1-3GlcNAcjβProNHCbz and the corresponding disaccharide analogue Galβ1-3GlcNAcαProNHCbz. LacβProNHCbz and LacNAcβProNHCbz were also tolerated with limited activity. His₆-AbGtr18 showed trace activity towards the other tested acceptors, including GlcNAcβProNHCbz, GalβpNP, type IV glycan Galβ1-3GalNAcβProNHCbz and type III or Core 1 glycan Galβ1-3GalNAcαProNHCbz.

TABLE 2
Acceptor substrate specificity of His₆-AbGtr18. ^{└a┘}

Yield (%)

| Acceptor | 15 min^{[b]} | 20 h^{[c]} |
|---|---|---|
| GalNAcα2AA | 35.7 ± 2.2 | 78.1 ± 0.6 |
| GalNAcαProNHCbz | 14.6 ± 1.1 | 82.5 ± 0.6 |
| GalNAcβProNHCbz | 1.7 ± 0.2 | 33.5 ± 0.8 |
| GlcNAcαProNHCbz | 0.5 ± 0.1 | 20.4 ± 0.1 |
| GlcNAcβProNHCbz | N.D.^{[d]} | 1.0 ± 0.1 |
| LacβProNHCbz | N.D. | 13.8 ± 0.2 |
| LacNAcβProNHCbz | N.D. | 7.7 ± 0.1 |
| Galβ1-3GalNAcαProNHCbz | N.D. | Trace^{[e]} |
| Galβ1-3GalNAcβProNHCbz | N.D. | Trace |
| Galβ1-3GlcNAcαProNHCbz | 1.8 ± 0.3 | 24.7 ± 0.5 |
| Galβ1-3GlcNAcβProNHCbz | 0.9 ± 0.1 | 23.9 ± 0.3 |
| GalαpNP | N.D. | 16.7 ± 0.2 |
| GalβpNP | N.D. | 1.6 ± 0.1 |

^{└a┘} Quantitative HPLC methods were used to determine the yields.
^{[b]}0.55 μM of His₆-AbGtr18 was used.
^{[c]}5.5 μM of His₆-AbGtr18 was used.
^{[d]}N.D.: not detected.
^{[e]}Activity confirmed by high-resolution mass spectrum.

Donor substrate specificity studies of MBP-AbGtr18-His₆ using in situ generated CMP-Leg5,7Ac₂, CMP-sialic acid, and derivatives were also conducted. A two-step process was established. In the first step, CMP-Leg5,7Ac₂, CMP-sialic acid, or their derivative was prepared from Leg5,7Ac₂, sialic acid, its derivative, or its precursor using a recombinant Legionella pneumophila CMP-Leg5,7Ac₂ synthetase (LpCLS) or Neisseria meningitidis CMP-sialic acid synthetase (NmCSS) with (when a nonulosonic acid precursor or its derivative was used as the starting material) or without (when a nonulosonic acid or its derivative was used as the starting material) Pasteurella multocida sialic acid aldolase (PmAldolase) and sodium pyruvate in the presence of cytidine 5′-triphosphate (CTP). In the second step, MBP-AbGtr18-His₆ sialylation reactions were carried out using GalNAcαProNHCbz as the acceptor substrate for 30 minutes or 20 hours and the reaction mixtures from the first step as the sources of CMP-Leg5,7Ac₂ and CMP-sialic acid donors. As shown in Table 3, in addition to CMP-Leg5,7Ac₂, MBP-AbGtr18-His₆ was also able to tolerate a variety of CMP-sialic acids and derivatives as donor substrates. CMP-Leg5,7Ac₂ was generated highly efficient from Leg5,7Ac₂, and was used efficiently by MBP-AbGtr18-His₆ which was also efficient in using in situ-generated CMP-Neu5Ac (generated from Neu5Ac or ManNAc), CMP-Neu5Az (generated from ManNAz) and CMP-Neu5Ac9N₃ (generated from ManNAc6N₃⁸). In comparison, CMP-Leg5,7diN₃ (generated from Leg5,7diN₃), CMP-Neu5Gc (generated from ManNGc) and CMP-Neu5Ac7N₃ (generated from ManNAc4N₃⁹) were suitable but less efficient donor substrates for MBP-AbGtr18-His₆. CMP-Neu5,7Ac₂ (generated from Man2,4diNAc), CMP-Neu5,7diN₃ (generated from Man2,4diN₃) and CMP-Neu5,7,9triN₃ (generated from Man2,4,6triN₃) can also be tolerated by MBP-AbGtr18-His₆ with limited activity. CMP-Kdn (generated from Kdn or Mannose), CMP-Neu5,7,9Ac3 (generated from Man2,4,6triNAc) and CMP-Neu5N₃ (generated from Man2N₃⁶) were poor donor substrates for MBP-AbGtr18-His₆.

TABLE 3
Donor substrate specificity of MBP-AbGtr18-His₆.

| | | Sialoside yield (%) | |
|---|---|---|---|
| Donor precursors | CMP-Sia Yield (%) | 0.5 h | 20 h |
| CMP-Leg5,7Ac₂ | — | 53.5 ± 0.5 | 72.6 ± 0.1 |
| Leg5,7Ac₂ | >99 | 56.6 ± 1.6 | 99.0 ± 0.6 |
| Leg5,7diN₃ | >99 | 44.3 ± 1.6 | 68.4 ± 1.5 |
| Neu5Ac | >99 | 47.4 ± 1.7 | 93.3 ± 0.6 |
| ManNAc | >99 | 37.6 ± 0.4 | 71.1 ± 0.4 |
| ManNGc | >99 | 13 ± 0.4 | 71.6 ± 2.5 |
| ManNAz | >99 | 57.3 ± 0.4 | 98.4 ± 1.8 |
| Kdn | >99 | N.D. | 9.6 ± 0.1 |
| Mannose | 77.7 ± 1.6 | N.D. | 5.0 ± 0.3 |
| Man2,4diNAc | 51.5 ± 0.9 | 5.5 ± 0.1 | 30.3 ± 0.3 |
| Man2,4,6triNAc | 24.1 ± 1.1 | N. D. | 2.8 ± 0.1 |
| ManNAc4N₃ | 84.4 ± 1.6 | 50.6 ± 0.5 | 72.5 ± 0.1 |
| ManNAc6N₃ | >99 | 55.5 ± 0.6 | 96.0 ± 2.1 |
| Man2,4diN₃ | 81.4 ± 0.3 | 28.0 ± 0.1 | 31.3 ± 1.1 |
| Man2,4,6triN₃ | 47.2 ± 0.1 | 20.0 ± 0.6 | 23.0 ± 1.0 |
| Man2N₃ | 57.6 ± 0.1 | N. D. | Trace |

### Example 5. Comparison of Legionaminic Acid Transferase with Sialyltransferases

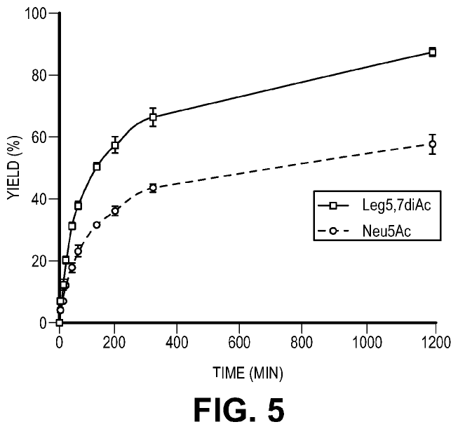

As indicated by the time-course studies using in situ generated CMP-Leg5,7Ac₂ and CMP-Neu5Ac (FIG. 5), MBP-AbGtr18-His₆ showed overall superior activity with CMP-Leg5,7Ac₂, suggesting its activity as a legionaminic acid transferase.

As indicated by activity assays using in situ generated CMP-Neu5Ac as a donor (Table 4), both MBP-AbGtr18-His₆ and Psp2,6ST(15-501)-His₆ A366G showed higher efficiency towards GalNAcαProNHCbz in 30 minutes when 1.2 M of catalysts was used. However, only MBP-AbGtr18-His₆ showed the highest efficiency for GalNAcαProNHCbz when the reaction time was extended to 20 hours with a yield of 74.1+0.5%. All catalysts except for MBP-AbGtr18-His₆ showed high efficiency in tolerating LacβProNHCbz with 40.8-66.8% yields in 15 minutes and 79.8-95.10% yields in 20 hours, respectively, when 0.4 μM of catalysts was used. MBP-AbGtr18-His₆ (0.4 μM) showed no detectable activity for LacβProNHCbz in 15 minutes and only 16.6±0.1% yield was obtained when the reaction time was extended to 20 hours, suggesting LacβProNHCbz as unfavorable acceptor.

TABLE 4
Activity comparison of MBP-AbGtr18-His₆ and α2-6-
sialyltransferases in transferring Neu5Ac to GalNAcαProNHCbz
and LacβProNHCbz, respectively.

| | Yield for | | Yield for | |
|---|---|---|---|---|
| | GalNAcαProNHCbz^{[a]} | | LacβProNHCbz^{[b]} | |
| Catalyst | 30 min | 20 h | 15 min | 20 h |
| MBP-AbGtr18-His₆ | 19.6 ± 1.8 | 74.1 ± 0.5 | N.D.^{[c]} | 16.6 ± 0.1 |
| Psp2,6ST(15-501)-His₆ | 6.5 ± 0.1 | 11.1 ± 0.2 | 40.8 ± 2.7 | 79.8 ± 0.8 |
| Psp2,6ST(15-501)-His₆ | 13.3 ± 0.3 | 19.3 ± 0.6 | 53.2 ± 0.9 | 95.1 ± 0.1 |
| A366G | | | | |
| His₆-Pd2,6ST(16-497) | 8.7 ± 0.3 | 14.5 ± 0.3 | 66.8 ± 0.4 | 85.4 ± 0.4 |

^{[a]}1.2 μM of catalyst was used.
^{[b]}0.4 μM of catalyst was used
^{[c]}N.D.: not detected.

When using in situ generated CMP-Leg5,7Ac₂ as a donor (Table 5), all catalysts except for MBP-AbGtr18-His₆ showed limited efficiency towards GalNAcαProNHCbz with 4.9-13.6% yields in 30 min and 10.9-17.7% yields in 20 h, respectively, when 6 M of catalysts was used. MBP-AbGtr18-His₆ showed the highest efficiency for GalNAcαProNHCbz when 6 M of enzyme (10-fold less) was used, with 26.5±0.1% yield in 30 min and 86.1±0.2% yield in 20 hours. His₆-Pd2,6ST(16-497) showed the highest efficiency for LacβProNHCbz in 30 minutes when 4 μM of enzyme was used with a yield of 33.4±0.5%. Only limited yields (4.0-7.0%) were obtained by other enzymes in 30 minutes. When the reaction time was extended to 20 hours, His₆-Pd2,6ST(16-497) showed moderate conversion yield of 43.5±1.8%, while MBP-AbGtr18-His₆ was less efficient with a yield of 35.1±0.8%. Psp2,6ST(15-501)-His₆ and its A366G mutant only showed very poor yields of 9.6-9.7%.

TABLE 5
Activity comparison of MBP-AbGtr18-His₆ and α2-6-
sialyltransferases in transferring Leg5,7Ac₂ to
GalNAcαProNHCbz and LacβProNHCbz, respectively.

| | Yields (%) | | | |
|---|---|---|---|---|
| | With | | With | |
| | GalNAcαProNHCbz^{[a]} | | LacβProNHCbz^{[b]} | |
| Enzymes | 30 min | 20 h | 30 min | 20 h |
| MBP-AbGtr18-His₆ | 26.5 ± 0.1 | 86.1 ± 0.2 | 7.0 ± 0.1 | 35.1 ± 0.8 |
| Psp2,6ST(15-501)- | 4.9 ± 0.2 | 10.9 ± 0.5 | 4.0 ± 0.3 | 9.7 ± 0.1 |
| His₆ | | | | |
| Psp2,6ST(15-501)- | 6.2 ± 0.1 | 11.0 ± 0.2 | 4.1 ± 0.1 | 9.6 ± 0.1 |
| His₆ | | | | |
| A366G | | | | |
| His₆-Pd2,6ST(16-497) | 13.6 ± 1.1 | 17.7 ± 0.1 | 33.4 ± 0.5 | 43.5 ± 1.8 |

^{[a]}0.6 μM of MBP-AbGtr18-His₆ and 6 μM of other enzymes were used, respectively.
^{[b]}4 μM of other enzymes was used.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

SEQUENCE LISTING
AbGtr18 (GenBank accession: AQQ74347.1)
SEQ ID NO: 1
MEHRQKSLIMCVTPLQILIAEKIIQSRPTEDFDVIIFALENNPKF
NYYAEKLKKYAKAYWYSYIVYKNSIYNLINFLKFICQFNFNGFNK
KYDNYYLSSIDSRYFQYILSKKTNSSNVFTFDDGTANIVKTSIYY
MKNINDEKKNKLFKILGIKYFQDDIKDMSKLHYTIYPEFKNIIEN
IKEVKLFNTIETSCQRDKEKNIFLGQPYEQLDKGLTIDKVERLMI
DLNIDFYFPHPREKYSPSNIEIIKTEKIFEDYIVNYLRNNKDICV
NIYTFMSSAALNVIAVEGVQVFFIQNKNLLNKYPDLFNILKDHKI
NFLKFKDI
AbGtr56_{K27} (GenBank accession: ALL34863.1)
SEQ ID NO: 2
MNLKKNKNLIICLTPLQMLIADSILRMKENEEYTFICICYGYNDK
YDLYFNRINDKVSSSFLYQVLSVNKIGRFFDLLRYKKNIVKKLDK
KFNNVYFASIDNPFVQLALSTVEYNNMISFDDGTANLWVNSIYNK
PVDRGVIQRIISKLVGISVNEEFIKARIFRHYTIFKNNMYLKGKS
EFLPLFNLDFEKEQNSKVIKIFLGQPLSDYNFPIFNKENVEYILK
ILDVDFYFPHPREKEEITSVETIKTRKIFEDYIVELLAKGHFVEV
YTFFSSAALTVANLQNVKVSAVVDNDTLNFFLELYNFFEMSGVQL
VELDNIREKRYNSNC
AbGtr56_{K44} (GenBank accession: QGW59139.1)
SEQ ID NO: 3
MNLKKNKNLIICLTPLQMLIADSILRMKENEEYTFICICYGYNDK
YDLYFNRINDKASYSFLYQVLSVNKIGRFFDLLRYKKNIVKKLDK
KFNNVYFASIDNPFVQLALSTVEYNNMISFDDGTANLWVNSIYNK
PVDRGVIQRITSKLVGISVNEEFIKARIFRHYTIFKNNMYLKGKS
EFLPLFNLDFEKEQNSKVIKIFLGQPLSDYNFPIFNKENVEYILK
ILDVDFYFPHPREKEEITSVETIKTRKIFEDYIVELLTLGYVVNV
YTFFSSAILTISGLENVQRFAVLDKQIFNHFSDLFAIFKENGIEL
IELDFDNANNL
AbGtr109 (GenBank accession: AWJ68081.1)
SEQ ID NO: 4
MEHRQKSLIMCVTPLQILIAEKIIQSRPAEDFDVIIFALENNPKF
NYY AEKLKKYAKAYWYSYIVYKSSIYNLINFLKFICQFNLNGFN
KKYDNYYLSSIDSRYFQYILSRKTNSSSVFTFDDGTANIVKTSIY
YMKNKNDENKNKLFRMLGIKYFQDDIKSMSKLHYTIYPDFENITS
YTQEIKLFDVEDSCNERKKILNIFLGQPYEQLSNHINLAKIKELL
EKLNINSYFPHPREKNTPDNIEVIYTNKIFEDYIINFLNKNKDTH
INIYTLMSSAALNVNSIDGLNVFFVRDDKILEKYSDFFKLLEYNG
INIISYEL
CjMaf4 (GenBank accession: ACC77728.1)
SEQ ID NO: 5
MTFTPTQKELFNKNIEALSNILLKESLKEIKSSKFELILGKDNLD
INLKDTSIKNNGGGYNENLLYQDPIKELQTMLNTYNDKYLLYPVL
YFYGFGNGILFKALLQNKNHQHIVVFEKNIEIIWIMFHILNFSSE
LQSARLMILENDKLQAQDYTELCSSKPFFQFSRIYFLELMSHYYE
RFHEDILGLNKKLAENFKNSIISHGNDSTDTLQGIEQFVYNLPSM
ITHPSYKELLSKRKGISDTAIIVSTGPSLTKQLPLLKKYANKATI
FCADSSYPILAKHGIKPDYVCMLERTEITAEFFNHDFGEFDKDIV
FVCAGVVHPKAIEYLKGRNRKYLIIPRYLYFPIYIKLKYFDFLYN
TPSVAHMACYLSLHLNHKNIIFIGQDLAY AENGNSHPDDYQNSA
NYESQMYEHILTEAYGGKKEIKTHEVWIFFKQILEAMIIKYHITT
YNCTEGGARIEGTIEKPFLWACENLLDKNLNKPFEKLEPLSLNKQ
NEFLLKAYYKVCKSIKHCRDFSKILSNDENNIQNIYLNLNKKEND
LNLAIRKIDEFKNKLENIKQMQDLYEILQPLRTQFELNLARIYVL
NPKTKEDAFNKSILWIKEHLEFMELVYGHIKAQENALIKNILPLE
EKLKERKLDKWMERVRR
CtWepA (GenBank accession: AFO84314.1)
SEQ ID NO: 6
MNKLEKYQLTPVCIVNNVHGLLIYYLYNTQYFFDTLFVVSDGISV
DVQKKLKHTIRIPSFSHSPKLLRIILRAIYYQLANFFLKFKKDNK
IVYGHDHLFYSQLFIKKAKRFILLEDGLANYSEHDAAKGGKIRKI
IFGSSGPFFGWSKQVSSVVLSGIVDIPDKLKNKTTLVDINMRWNE
LAPQQKQRFINIFSAEPYIALKKVVIFTQPFSEDAMMSEAQKINI
YKKIYNHYRQIFQKDEICIKSHPREHTDYSQYFDCVFIKSKIPGQ
IMILNDRPEILVTIYSSVGYIRDNIKTHIWGTEFDSFLLEKVGYF
EGNYTGWTSNE
EcWeiF (GenBank accession: ADJ19202.1)
SEQ ID NO: 7
MGNIFIVESPFQLSNALLYHKKNDSVIVRLNGENKNDFQIEKMLS
SFNGKVYIKKASKESKFDLIRFVLFFAVPVLIANLNKKVIIGNYN
SLWMRVMGYLFNPFHFAVLDDGLITIRTIKRLDDNISRSGSIKKR
FLLLLAPRFITQYKIYSNFIQIYNQEINKRKRTTRAIKAGRVCFI
GSPLFDKNVLTFDFYVKCLAAISDNLKRCGYSIEYYPHRSEKNIS
YLNVFFDDVIKSDDSIKVYYSASNELPEIFVSFYSSALLNLRSDY
PECKFISYKLDCNEINGKFRYEIMEAYNFLAFSGIEVVTI
EcWeiK (GenBank accession: ADJ19216.1)
SEQ ID NO: 8
MLQKTNKKGFQLALVESLLQLKTLDSYSGNNKNNIHLFVRLNGEQ
KNEEEILNFIKPRACHYSSVQFVSIRRNDKFSLLFNILKLRLFLF
CKRKVILIIGDPRALWMNMISSFKNVHDVIYLEDGMSTVLFYQTF
KPKYPHKHYKLVTRLKLDGNAFLSLIPLEVKKNTVMRIDNDVALF
IGMPMIENNALSKKKYLSYLHKIIMSLKNMKITKFYYAPHRYENE
NNFYLYENLGFHMLDTDCAIEDYLNSKNIIPAVYASFYSTALLQI
DTLFYGVSVICYVINVEELNYDFRNPALYAYEYYNKTPSIIKVDL
HD
His₆-AbFnlA
SEQ ID NO: 9
MGSSHHHHHHSSGLVPRGSHMASMTGGQQMGRGSMFKDKVLLITGG
TGSFGNAVLKRFLETDIKEIRIFSRDEKKQDDMRKKYHSAKLKFY
IGDVRDYNSILNATRGVDYIYHAAALKQVPSCEFHPMEAVKTNVL
GTENVLEAAIQNHVKRVVCLSTDKAVYPINAMGISKAMMEKVMVA
KSRNLEGLNTVICGTRYGNVMASRGSVIPLFVDQIRQGKPLTITD
PNMTRFMMTLEDAVDLVLYAFEHGENGDIFVQKAPAATIAVLAEA
LKQLLNVEDHPISIMGTRHGEKAFEALLSREEMVHAFDQGDYFRV
PADQRDLNYEKYVEDGDLKITEFEDYNSHNTTRLDVEGMKQLLLK
LDFVRALTRGEYISPEA
AbFnlA
SEQ ID NO: 10
MFKDKVLLITGGTGSFGNAVLKRFLETDIKEIRIFSRDEKKQDDM
RKKYHSAKLKFYIGDVRDYNSILNATRGVDYIYHAAALKQVPSCE
FHPMEAVKTNVLGTENVLEAAIQNHVKRVVCLSTDKAVYPINAMG
ISKAMMEKVMVAKSRNLEGLNTVICGTRYGNVMASRGSVIPLFVD
QIRQGKPLTITDPNMTRFMMTLEDAVDLVLYAFEHGENGDIFVQK
APAATIAVLAEALKQLLNVEDHPISIMGTRHGEKAFEALLSREEM
VHAFDQGDYFRVPADQRDLNYEKYVEDGDLKITEFEDYNSHNTTR
LDVEGMKQLLLKLDFVRALTRGEYISPEA
His₆-AbFnlB
SEQ ID NO: 11
MGSSHHHHHHSSGLVPRGSHMASMTGGQQMGRGSMKVLVTGSNGF
IAKNFIQFLSEKPEVEILKAHRETTDQEFEQVVLAADWIVHLAGV
NRPLNEEEFAEGNTTLTEKISQILQQANKKTPVILSSSIQVERDN
PYGKSKLGGEQALVTLHQAQGNPVYICRLANVFGKWSRPNYNSAV
ATFCHNVANDLPLQIHDENAVIRLVYVDDVVETFWNILNGQQVEQ
FFQVEPEYQITVGDLAKVLNGFKASRGTLITDRVGTGLTRALYST
YLSFLKPEQFDYSVPKYGDARGVFVEMLKTPDAGQFSYFTAHPGI
TRGGHYHHTKTEKFLVIKGKALFKFKHVVTGEFYELETHGDEPRI
VETVPGWTHDITNIGDEEMVVMLWANEIFDRNKPDTYAMPITN
AbFnlB
SEQ ID NO: 12
MKVLVTGSNGFIAKNFIQFLSEKPEVEILKAHRETTDQEFEQVVL
AADWIVHLAGVNRPLNEEEFAEGNTTLTEKISQILQQANKKTPVI
LSSSIQVERDNPYGKSKLGGEQALVTLHQAQGNPVYICRLANVFG
KWSRPNYNSAVATFCHNVANDLPLQIHDENAVIRLVYVDDVVETF
WNILNGQQVEQFFQVEPEYQITVGDLAKVLNGFKASRGTLITDRV
GTGLTRALYSTYLSFLKPEQFDYSVPKYGDARGVFVEMLKTPDAG
QFSYFTAHPGITRGGHYHHTKTEKFLVIKGKALFKFKHVVTGEFY
ELETHGDEPRIVETVPGWTHDITNIGDEEMVVMLWANEIFDRNKP
DTYAMPITN
His₆ tag
SEQ ID NO: 13
HHHHHH
MBP-AbGtr18-His₆ Forward Primer
SEQ ID NO: 14
GTTTTTGAATTCATGGAACATCGTCAAAAGAG
MBP-AbGtr18-His₆ Reverse Primer
SEQ ID NO: 15
GTTTTTTGTCGACTCAATGATGATGATGATGATGGATGTCCTTGA
ACTTCAGAA

1. A method for preparing a glycan product containing a nonulosonic acid moiety, the method comprising:
(i) forming a reaction mixture comprising a legionaminic acid transferase (LegT), a donor comprising a nonulosonic acid moiety, and a glycan acceptor, and
(ii) maintaining the reaction mixture under conditions for LegT-catalyzed transfer of the nonulosonic acid moiety from the donor to the glycan acceptor, thereby forming the glycan product containing the nonulosonic acid moiety.

2. The method of claim 1, wherein the LegT is a Legionella LegT, an Acinetobacter LegT, a Campylobacter LegT, a Cronobacter LegT, an Enterobacter LegT, an Escherichia LegT, an L. pneumophila LegT, an A. baumannii LegT, a C. jejuni LegT, a C. turicensis LegT, an E. cloacae LegT, or an E. coli LegT.

3. (canceled)

4. The method of claim 2, wherein the LegT comprises a polypeptide having at least 80% sequence identity to SEQ ID NO:1 (A. baumannii K8 AbGtr18), SEQ ID NO:2 (A. baumannii K27 AbGtr56K27), SEQ ID NO:3 (A. baumannii K44 AbGtr56K44), SEQ ID NO:4 (A. baumannii K54 AbGtr109), SEQ ID NO:5 (C. jejuni 108 CjMaf4), SEQ ID NO:6 (C. turicensis G3882 CtWepA), SEQ ID NO:7 (E. coli O161 EcWeiF), or SEQ ID NO:8 (E. coli O61 EcWeiK).

5. (canceled)

6. The method of claim 4, wherein the N-terminus of the polypeptide is fused to a maltose binding protein.

7. The method of claim 1, wherein the nonulosonic acid moiety is a legionaminic acid selected from the group consisting of 5,7-diacetamido-3,5,7,9-tetradeoxy-D-glycero-D-galacto-non-2-ulosonic acid (Leg5,7Ac2), 4-epi-legionaminic acid, 8-epi-legionaminic acid, Leg5Ac7Ac4OAc, Leg5Ac7Hb, pseudaminic acid (Pse), Pse5Ac7Ac, Pse5Ac7Ac4OAc, Pse5Ac7Hb, acinetaminic acid, and fusaminic acid, wherein Ac is acetyl and Hb is 3-hydroxybutanoyl.

8. The method of claim 1, wherein the nonulosonic acid is a sialic acid selected from the group consisting of neuraminic acid, Neu5Ac, Neu5Gc, and Kdn.

9. The method of claim 1, wherein the nonulosonic acid moiety is selected from the group consisting of Leg5,7Ac₂, Leg5,7diN₃, Neu5Ac, Neu5Gc, Kdn, Neu5Az, Neu5Ac9N₃, Neu5Ac7N₃, Neu5,7Ac₂, Neu5,7diN₃, Neu5,7,9triN₃, Leg5Ac7N₃, and Leg5N₃7Ac, and wherein the donor is selected from the group consisting of CMP-Leg5,7Ac₂, CMP-Leg5,7diN₃, CMP-Neu5Ac, CMP-Neu5Gc, CMP-Kdn, CMP-Neu5Az, CMP-Neu5Ac9N₃, CMP-Neu5Ac7N₃, CMP-Neu5,7Ac₂, CMP-Neu5,7diN₃, CMP-Neu5,7,9triN₃, CMP-Leg5Ac7N₃, and CMP-Leg5N₃7Ac.

10. (canceled)

11. The method of claim 1, wherein the nonulosonic acid moiety is a legionaminic acid, and wherein forming the reaction mixture comprises combining the legionaminic acid with cytidine 5′-triphosphate and a CMP-legionaminic acid synthetase (CLS) to form the donor, and wherein the CLS is Legionella pneumophila CMP-Leg5,7Ac₂ synthetase (LpCLS).

12. (canceled)

13. The method of claim 1, wherein the nonulosonic acid moiety is a sialic acid, and wherein forming the reaction mixture comprises combining the sialic acid with cytidine 5′-triphosphate and a CMP-sialic acid synthetase (CSS) to form the donor, wherein the CSS is Neisseria meningitidis CMP-sialic acid synthetase (NmCSS).

14. (canceled)

15. The method of claim 13, wherein forming the reaction mixture further comprises combining mannose, a mannosamine, an N-acetylmannosamine, or an azido-mannose with pyruvate and a sialic acid aldolase to form the sialic acid, and wherein the sialic acid aldolase is Pasteurella multocida sialic acid aldolase (PmAldolase).

16. (canceled)

17. The method of claim 1, wherein the glycan acceptor is an N-acetylgalactosamine or an N-acetylgalactosamine-terminated glycoside “GalNAc-OR,” wherein R is H, a monosaccharide, an oligosaccharide, a polysaccharide, a peptide, a protein, a lipid, a glycopeptide, a glycoprotein, or a glycolipid.

18. The method of claim 17, wherein the N-acetylgalactosamine is linked to OR via an α-linkage at the anomeric carbon C-1 of GalNAc.

19. The method of claim 1, wherein the glycan acceptor comprises a methyl 2-anthranilic acid ester moiety, a benzyloxycarbonyl-propylamine moiety, or 4-nitrophenyl moiety at the reducing end of the glycan acceptor.

20. The method of claim 1, wherein the glycan acceptor comprises one or more L-FucNAc moieties.

21. The method of claim 1, wherein the glycan product comprises one or more A. baumannii bacterial capsular polysaccharide K2 units, K6 units, K7 units, K8 units, K16 units, K27 units, K33 units, K42 units, K44 units, K46 units, K54 units, K63 units, K90 units, or K93 units.

22. The method of claim 1, wherein the reaction mixture is maintained at a temperature ranging from about 20° C. to about 40° C. in step (ii), and wherein the reaction mixture is maintained in step (ii) for a period of time ranging from about 5 minutes to about 48 hours.

23. (canceled)

24. (canceled)

25. The method of claim 11, wherein forming the reaction mixture further comprises combining a nonulosonic acid precursor with pyruvate and a sialic acid aldolase to form the nonulosonic acid, wherein the nonulosonic acid precursor is selected from the group consisting of 6deoxyMan2,4diNAc, 6deoxyMan2N34NAc, 6deoxyManNAc4N3, and Man2,4diNAc, and wherein the sialic acid aldolase is Pasteurella multocida sialic acid aldolase (PmAldolase).

26. (canceled)

27. (canceled)

28. The method of claim 13, wherein forming the reaction mixture further comprises combining a nonulosonic acid precursor with pyruvate and a sialic acid aldolase to form the nonulosonic acid, and wherein the nonulosonic acid precursor is selected from the group consisting of ManNAc, ManNGc, Mannose, ManNAz, ManNAc6N3, ManNAc4N3, Man2,4diN3, Man2,4,6triN3, 6deoxyMan2,4diN3, and 6deoxyManNAc4N3.

29. (canceled)

30. A glycan product prepared according to the method of claim 1.

31. A legionaminic acid transferase fusion protein comprising a polypeptide having at least 80% sequence identity to SEQ ID NO:1, wherein the N-terminus of the polypeptide is fused to a maltose binding protein.

32. The legionaminic acid transferase fusion protein according to claim 31, wherein the maltose binding protein comprises one or more valine substitutions.

33. An isolated nucleic acid encoding legionaminic acid transferase fusion protein according to claim 31.

34. A vector comprising the isolated nucleic acid of claim 33.

35. A host cell comprising the vector of claim 34.


SEQUENCE LISTING (15 sequences)

SEQ ID NO: 1 - protein, 323 aa, Synthetic construct
MEHRQKSLIMCVTPLQILIAEKIIQSRPTEDFDVIIFALENNPKFNYYAEKLKKYAKAYWYSYIVYKNSIYNLINFLKFICQFNFNGFNKKYDNYYLSSIDSRYFQYILSKKTNSSNVFTFDDGTANIVKTSIYYMKNINDEKKNKLFKILGIKYFQDDIKDMSKLHYTIYPEFKNIIENIKEVKLFNTIETSCQRDKEKNIFLGQPYEQLDKGLTIDKVERLMIDLNIDFYFPHPREKYSPSNIEIIKTEKIFEDYIVNYLRNNKDICVNIYTFMSSAALNVIAVEGVQVFFIQNKNLLNKYPDLFNILKDHKINFLKFKDI

SEQ ID NO: 2 - protein, 330 aa, Synthetic construct
MNLKKNKNLIICLTPLQMLIADSILRMKENEEYTFICICYGYNDKYDLYFNRINDKVSSSFLYQVLSVNKIGRFFDLLRYKKNIVKKLDKKFNNVYFASIDNPFVQLALSTVEYNNMISFDDGTANLWVNSIYNKPVDRGVIQRIISKLVGISVNEEFIKARIFRHYTIFKNNMYLKGKSEFLPLFNLDFEKEQNSKVIKIFLGQPLSDYNFPIFNKENVEYILKILDVDFYFPHPREKEEITSVETIKTRKIFEDYIVELLAKGHFVEVYTFFSSAALTVANLQNVKVSAVVDNDTLNFFLELYNFFEMSGVQLVELDNIREKRYNSNC

SEQ ID NO: 3 - protein, 326 aa, Synthetic construct
MNLKKNKNLIICLTPLQMLIADSILRMKENEEYTFICICYGYNDKYDLYFNRINDKASYSFLYQVLSVNKIGRFFDLLRYKKNIVKKLDKKFNNVYFASIDNPFVQLALSTVEYNNMISFDDGTANLWVNSIYNKPVDRGVIQRITSKLVGISVNEEFIKARIFRHYTIFKNNMYLKGKSEFLPLFNLDFEKEQNSKVIKIFLGQPLSDYNFPIFNKENVEYILKILDVDFYFPHPREKEEITSVETIKTRKIFEDYIVELLTLGYVVNVYTFFSSAILTISGLENVQRFAVLDKQIFNHFSDLFAIFKENGIELIELDFDNANNL

SEQ ID NO: 4 - protein, 322 aa, Synthetic construct
MEHRQKSLIMCVTPLQILIAEKIIQSRPAEDFDVIIFALENNPKFNYYAEKLKKYAKAYWYSYIVYKSSIYNLINFLKFICQFNLNGFNKKYDNYYLSSIDSRYFQYILSRKTNSSSVFTFDDGTANIVKTSIYYMKNKNDENKNKLFRMLGIKYFQDDIKSMSKLHYTIYPDFENITSYTQEIKLFDVEDSCNERKKILNIFLGQPYEQLSNHINLAKIKELLEKLNINSYFPHPREKNTPDNIEVIYTNKIFEDYIINFLNKNKDTHINIYTLMSSAALNVNSIDGLNVFFVRDDKILEKYSDFFKLLEYNGINIISYEL

SEQ ID NO: 5 - protein, 646 aa, Synthetic construct
MTFTPTQKELFNKNIEALSNILLKESLKEIKSSKFELILGKDNLDINLKDTSIKNNGGGYNENLLYQDPIKELQTMLNTYNDKYLLYPVLYFYGFGNGILFKALLQNKNHQHIVVFEKNIEIIWIMFHILNFSSELQSARLMILENDKLQAQDYTELCSSKPFFQFSRIYFLELMSHYYERFHEDILGLNKKLAENFKNSIISHGNDSTDTLQGIEQFVYNLPSMITHPSYKELLSKRKGISDTAIIVSTGPSLTKQLPLLKKYANKATIFCADSSYPILAKHGIKPDYVCMLERTEITAEFFNHDFGEFDKDIVFVCAGVVHPKAIEYLKGRNRKYLIIPRYLYFPIYIKLKYFDFLYNTPSVAHMACYLSLHLNHKNIIFIGQDLAYAENGNSHPDDYQNSANYESQMYEHILTEAYGGKKEIKTHEVWIFFKQILEAMIIKYHITTYNCTEGGARIEGTIEKPFLWACENLLDKNLNKPFEKLEPLSLNKQNEFLLKAYYKVCKSIKHCRDFSKILSNDFNNIQNIYLNLNKKENDLNLAIRKIDEFKNKLENIKQMQDLYEILQPLRTQFELNLARIYVLNPKTKEDAFNKSILWIKEHLEFMELVYGHIKAQENALIKNILPLEEKLKERKLDKWMERVRR

SEQ ID NO: 6 - protein, 326 aa, Synthetic construct
MNKLEKYQLTPVCIVNNVHGLLIYYLYNTQYFFDTLFVVSDGISVDVQKKLKHTIRIPSFSHSPKLLRIILRAIYYQLANFFLKFKKDNKIVYGHDHLFYSQLFIKKAKRFILLEDGLANYSEHDAAKGGKIRKIIFGSSGPFFGWSKQVSSVVLSGIVDIPDKLKNKTTLVDINMRWNELAPQQKQRFINIFSAEPYIALKKVVIFTQPFSEDAMMSEAQKINIYKKIYNHYRQIFQKDEICIKSHPREHTDYSQYFDCVFIKSKIPGQIMILNDRPEILVTIYSSVGYIRDNIKTHIWGTEFDSFLLEKVGYFEGNYTGWTSNE

SEQ ID NO: 7 - protein, 310 aa, Synthetic construct
MGNIFIVESPFQLSNALLYHKKNDSVIVRLNGENKNDFQIEKMLSSFNGKVYIKKASKESKFDLIRFVLFFAVPVLIANLNKKVIIGNYNSLWMRVMGYLFNPFHFAVLDDGLITIRTIKRLDDNISRSGSIKKRFLLLLAPRFITQYKIYSNFIQIYNQEINKRKRTTRAIKAGRVCFIGSPLFDKNVLTFDFYVKCLAAISDNLKRCGYSIEYYPHRSEKNISYLNVFFDDVIKSDDSIKVYYSASNELPEIFVSFYSSALLNLRSDYPECKFISYKLDCNEINGKFRYEIMEAYNFLAFSGIEVVTI

SEQ ID NO: 8 - protein, 317 aa, Synthetic construct
MLQKTNKKGFQLALVESLLQLKTLDSYSGNNKNNIHLFVRLNGEQKNEEEILNFIKPRACHYSSVQFVSIRRNDKFSLLFNILKLRLFLFCKRKVILIIGDPRALWMNMISSFKNVHDVIYLEDGMSTVLFYQTFKPKYPHKHYKLVTRLKLDGNAFLSLIPLEVKKNTVMRIDNDVALFIGMPMIENNALSKKKYLSYLHKIIMSLKNMKITKFYYAPHRYENENNFYLYENLGFHMLDTDCAIEDYLNSKNIIPAVYASFYSTALLQIDTLFYGVSVICYVINVEELNYDFRNPALYAYEYYNKTPSIIKVDLHD

SEQ ID NO: 9 - protein, 378 aa, Synthetic construct
MGSSHHHHHHSSGLVPRGSHMASMTGGQQMGRGSMFKDKVLLITGGTGSFGNAVLKRFLETDIKEIRIFSRDEKKQDDMRKKYHSAKLKFYIGDVRDYNSILNATRGVDYIYHAAALKQVPSCEFHPMEAVKTNVLGTENVLEAAIQNHVKRVVCLSTDKAVYPINAMGISKAMMEKVMVAKSRNLEGLNTVICGTRYGNVMASRGSVIPLFVDQIRQGKPLTITDPNMTRFMMTLEDAVDLVLYAFEHGENGDIFVQKAPAATIAVLAEALKQLLNVEDHPISIMGTRHGEKAFEALLSREEMVHAFDQGDYFRVPADQRDLNYEKYVEDGDLKITEFEDYNSHNTTRLDVEGMKQLLLKLDFVRALTRGEYISPEA

SEQ ID NO: 10 - protein, 344 aa, Synthetic construct
MFKDKVLLITGGTGSFGNAVLKRFLETDIKEIRIFSRDEKKQDDMRKKYHSAKLKFYIGDVRDYNSILNATRGVDYIYHAAALKQVPSCEFHPMEAVKTNVLGTENVLEAAIQNHVKRVVCLSTDKAVYPINAMGISKAMMEKVMVAKSRNLEGLNTVICGTRYGNVMASRGSVIPLFVDQIRQGKPLTITDPNMTRFMMTLEDAVDLVLYAFEHGENGDIFVQKAPAATIAVLAEALKQLLNVEDHPISIMGTRHGEKAFEALLSREEMVHAFDQGDYFRVPADQRDLNYEKYVEDGDLKITEFEDYNSHNTTRLDVEGMKQLLLKLDFVRALTRGEYISPEA

SEQ ID NO: 11 - protein, 403 aa, Synthetic construct
MGSSHHHHHHSSGLVPRGSHMASMTGGQQMGRGSMKVLVTGSNGFIAKNFIQFLSEKPEVEILKAHRETTDQEFEQVVLAADWIVHLAGVNRPLNEEEFAEGNTTLTEKISQILQQANKKTPVILSSSIQVERDNPYGKSKLGGEQALVTLHQAQGNPVYICRLANVFGKWSRPNYNSAVATFCHNVANDLPLQIHDENAVIRLVYVDDVVETFWNILNGQQVEQFFQVEPEYQITVGDLAKVLNGFKASRGTLITDRVGTGLTRALYSTYLSFLKPEQFDYSVPKYGDARGVFVEMLKTPDAGQFSYFTAHPGITRGGHYHHTKTEKFLVIKGKALFKFKHVVTGEFYELETHGDEPRIVETVPGWTHDITNIGDEEMVVMLWANEIFDRNKPDTYAMPITN

SEQ ID NO: 12 - protein, 369 aa, Synthetic construct
MKVLVTGSNGFIAKNFIQFLSEKPEVEILKAHRETTDQEFEQVVLAADWIVHLAGVNRPLNEEEFAEGNTTLTEKISQILQQANKKTPVILSSSIQVERDNPYGKSKLGGEQALVTLHQAQGNPVYICRLANVFGKWSRPNYNSAVATFCHNVANDLPLQIHDENAVIRLVYVDDVVETFWNILNGQQVEQFFQVEPEYQITVGDLAKVLNGFKASRGTLITDRVGTGLTRALYSTYLSFLKPEQFDYSVPKYGDARGVFVEMLKTPDAGQFSYFTAHPGITRGGHYHHTKTEKFLVIKGKALFKFKHVVTGEFYELETHGDEPRIVETVPGWTHDITNIGDEEMVVMLWANEIFDRNKPDTYAMPITN

SEQ ID NO: 13 - protein, 6 aa, Synthetic construct
HHHHHH

SEQ ID NO: 14 - DNA, 32 bp, Synthetic construct
  source 1..32: mol_type = other DNA
gtttttgaattcatggaacatcgtcaaaagag

SEQ ID NO: 15 - DNA, 54 bp, Synthetic construct
  source 1..54: mol_type = other DNA
gttttttgtcgactcaatgatgatgatgatgatggatgtccttgaacttcagaa